(19) United States
(12) Patent Application Publication
(10) Pub. No.: US 2023/0032136 A1
(43) Pub. Date: Feb. 2, 2023

(54) METHOD FOR DETERMINATION OF 3D GENOME ARCHITECTURE WITH BASE PAIR RESOLUTION AND FURTHER USES THEREOF

(71) Applicants: Suhas RAO, Houston, TX (US); Andreas GNIRKE, Cambridge, MA (US); Saul GODINEZ, Houston, TX (US); Huiya GU, Houston, TX (US); Elena STAMENOVA, Cambridge, MA (US); THE BROAD INSTITUTE, INC., CAMBRIDGE, MA (US); BAYLOR COLLEGE OF MEDICINE, HOUSTON, MA (US); THE BOARD OF TRUSTEES OF THE LELAND STANFORD JUNIOR UNIVERSITY, Stanford, CA (US)

(72) Inventors: Eric S. LANDER, Cambridge, MA (US); Erez AIDEN, Houston, TX (US); Huiya GU, Houston, TX (US); Saul GODINEZ PULIDO, Houston, TX (US); Suhas RAO, Stanford, CA (US); Elena STAMENOVA, Cambridge, MA (US); Namita MITRA, Houston, TX (US)

(21) Appl. No.: 17/785,042

(22) PCT Filed: Dec. 11, 2020
(86) PCT No.: PCT/US2020/064704

§ 371 Date: Jun. 13, 2022

Related U.S. Application Data
(60) Provisional application No. 62/948,191, filed on Dec. 13, 2019.
(60) Provisional application No. 62/948,312, filed on Dec. 15, 2019.

(51) Int. Cl. C12Q 1/6841; C12Q 1/6806

(52) U.S. Cl. CPC C12Q 1/6841; C12Q 1/6806; C12Q 2521/301; C12Q 2521/501; C12Q 2523/101; C12Q 2535/122

(57) ABSTRACT

Disclosed are methods for detecting spatial proximity relationships between nucleic acid sequences, such as genomic DNA, in a cell. The method includes providing a sample of one or more crosslinked cells comprising nucleic acids; permeabilizing isolated nuclei under conditions that preserve contacts; fragmenting the nucleic acids present in the nuclei; filling in and repairing the ends with at least one labeled nucleotide; joining the filled in end of the fragmented nucleic acids that are in close physical proximity to create one or more end joined nucleic acid fragments having a junction; isolating the one or more end joined nucleic acid fragments using the labeled nucleotide; and determining the sequence at the junction of the one or more end joined nucleic acid fragments.

### CROSS-REFERENCE TO RELATED APPLICATIONS

This application claims the benefit of U.S. Provisional Application Nos. 62/948,191, filed Dec. 13, 2019, and 62/948,312, filed Dec. 15, 2019. The entire contents of the above-identified applications are hereby fully incorporated herein by reference.

### STATEMENT REGARDING FEDERALLY SPONSORED RESEARCH

This invention was made with government support under Grant Nos. 0D008540, HG006193, and HG003067 awarded by the National Institutes of Health, and Grant No. PHY1427654 awarded by the National Science Foundation. The government has certain rights in the invention.

### REFERENCE TO AN ELECTRONIC SEQUENCE LISTING

The contents of the electronic sequence listing (“BROD-5070WP ST25.txt”; Size is 12,608 bytes and it was created on Dec. 10, 2020) is herein incorporated by reference in its entirety.

### TECHNICAL FIELD

The subject matter disclosed herein is generally directed to methods for identifying nucleic acids in close proximity within a cell or system.

### BACKGROUND

It has been suggested that the three-dimensional structure of nucleic acids in a cell may be involved in complex biological regulation, for example compartmentalizing the nucleus and bringing widely separated functional elements into close spatial proximity. Understanding how nucleic acids interact, and perhaps more importantly how this interaction, or lack thereof, regulates cellular processes, presents a new frontier of exploration. For example, understanding chromosomal folding and the patterns therein can provide insight into the complex relationships between chromatin structure, gene activity, and the functional state of the cell. Adding ribonucleic acids (RNAs) into the mix adds a further level of complexity.

Typically, deoxyribonucleic acid (DNA) is viewed as a linear molecule, with little attention paid to the three-dimensional organization. However, chromosomes are not rigid, and while the linear distance between two genomic loci indeed may be vast, when folded, the special distance may be small. For example, while regions of chromosomal DNA may be separated by many megabases, they also can be immediately adjacent in 3-dimensional space. Much the same way a protein can fold to bring sequence elements together to form an active site, from the standpoint of gene regulation, long-range interactions between genomic loci may form active centers. For example, gene enhancers, silencers, and insulator elements might function across vast genomic distances.

The existence of long-range interactions complicates efforts to understand the pathways that regulate cellular processes, because the interacting regulatory elements could lie at a great genomic distance from a target gene, even on another chromosome. In the case of oncogenes and other disease-associated genes, identification of long-range genetic regulators would be of great use in identifying the genomic variants responsible for the disease state and the process by which the disease state is brought about.

Citation or identification of any document in this application is not an admission that such a document is available as prior art to the present invention.

### SUMMARY

In one aspect, the present invention provides for an in situ method for detecting spatial proximity relationships between genomic DNA in a cell with base pair resolution, comprising: providing a sample of one or more cells; crosslinking the cells with a chemical crosslinker; lysing the cells to obtain isolated nuclei; permeabilizing the nuclei under conditions that preserve cohesin complex integrity in the crosslinked cells; enzymatically fragmenting the chromatin present in the nuclei; performing end repair and/or fill-in on the ends of the chromatin fragments with at least one labeled nucleotide, wherein the labeled nucleotide is capable of being used to isolate the chromatin fragments; ligating the repaired and/or filled in ends of the chromatin fragments that are in close physical proximity to create one or more end joined nucleic acid fragments having one or more junctions, wherein the site of the one or more junctions comprises one or more labeled nucleic acids; reversing the crosslinking; isolating the one or more end joined nucleic acid fragments using the labeled nucleotide; and sequencing at the one or more junctions of the one or more end joined nucleic acid fragments by using ligation junction sequencing, thereby detecting spatial proximity relationships between genomic DNA in a cell.

In certain embodiments, the steps of enzymatically fragmenting the chromatin present in the nuclei, performing end repair and/or fill-in on the ends of the chromatin fragments with at least one labeled nucleotide, and ligating the repaired and/or filled in ends of the chromatin fragments comprise:

a. a serial process comprising: i. digesting the chromatin with a first restriction enzyme; ii. filling in the overhanging ends produced from (i); iii. ligating the filled in end of the chromatin fragments from (ii); iv. digesting the chromatin fragments from (iii) with a second restriction enzyme; v. filling in the overhanging ends produced from (iv); and vi. ligating the filled in end of the chromatin fragments from (v);

b. a single-step process comprising: i. in a single-step, fragmenting the chromatin present in the cells by contacting the chromatin with two restriction enzymes, filling in one or more overhanging ends of the chromatin fragments, and ligating two or more filled in ends;

c. a parallel process comprising: i. fragmenting the chromatin present in the cell with two restriction enzymes in the same or parallel reactions; ii. filling in the overhanging ends from (i), wherein the optional parallel reaction are optionally combined; and iii. ligating two or more filled ends from (ii), wherein the optional parallel reaction are optionally combined;

d. a first MNase process comprising: i. fragmenting the chromatin using micrococcal nuclease (MNase); ii. repairing one or more overhanging ends produced in (i); filling in one or more repaired overhanging ends from (ii); iv. ligating two or more filled ends from (iii);

e. a second MNase process comprising: i. fragmenting the chromatin present in the cells with MNase; ii. in a single step, repairing one or more ends of the chromatin fragments from (i), filling in one or more repaired overhanging ends from (ii), and ligating two or more filled ends from (i); or

f. a third MNase process comprising: i. in a single step, fragmenting the chromatin present in the cells with MNase, repairing one or more ends of the chromatin fragments, filling in one or more repaired overhanging ends, and ligating two or more filled ends.

In certain embodiments, ligation junction sequencing comprises selecting and sequencing approximately 250 base pair fragments using paired end sequencing. In certain embodiments, ligation junction sequencing comprises selecting and sequencing approximately 300 base pair fragments from a single end. In certain embodiments, the nuclei are permeabilized by a method comprising NP40, digitonin, tween, streptolysin, exonuclease 1 buffer and pepsin, cationic lipids, hypotonic shock, or ultrasonication; and wherein SDS is not used. In certain embodiments, the method further comprises determining the sequence of a loop anchor with at least 10 base pair resolution. In certain embodiments, the method further comprises identifying a sequence motif bound by a protein within 50 base pairs outside of the loop anchor. In certain embodiments, a promoter element bound by an RNA polymerase is identified. In certain embodiments, an enhancer motif bound by a transcription factor is identified. In certain embodiments, the method further comprises identifying CTCF independent loops wherein cohesin is arrested by a factor other than CTCF. In certain embodiments, cohesin is arrested by an RNA polymerase or a transcription factor. In certain embodiments, promoter/enhancer loops are identified. In certain embodiments, the method further comprises identifying sequence variants in an enhancer element and linking the variant to a gene. In certain embodiments, the method further comprises determining the whole genome sequence for the cell based on the determined sequence information. In certain embodiments, the method further comprises determining the whole exome sequence for the cell by enriching for exome sequences in the joined DNA fragments.

In another aspect, the present invention provides for an in situ method for detecting spatial proximity relationships between genomic DNA in in a cell, comprising: providing a sample of one or more cells; crosslinking the cells with a chemical crosslinker; lysing the cells to obtain isolated nuclei; permeabilizing the nuclei; enzymatically fragmenting the chromatin present in the nuclei; performing end repair and/or fill-in on the ends of the chromatin fragments with at least one labeled nucleotide, wherein the labeled nucleotide is capable of being used to isolate the chromatin fragments; ligating the repaired and/or filled in ends of the chromatin fragments that are in close physical proximity to create one or more end joined nucleic acid fragments having one or more junctions, wherein the site of the one or more junctions comprises one or more labeled nucleic acids; reversing the crosslinking; isolating the one or more end joined nucleic acid fragments using the labeled nucleotide; and sequencing at the one or more junctions of the one or more end joined nucleic acid fragments, thereby detecting spatial proximity relationships between genomic DNA in a cell,

wherein the steps of enzymatically fragmenting the chromatin present in the nuclei, performing end repair and/or fill-in on the ends of the chromatin fragments with at least one labeled nucleotide, and ligating the repaired and/or filled in ends of the chromatin fragments comprise:

a. a serial process comprising: i. digesting the chromatin with a first restriction enzyme; ii. filling in the overhanging ends produced from (i); iii. ligating the filled in end of the chromatin fragments from (ii); iv. digesting the chromatin fragments from (iii) with a second restriction enzyme; v. filling in the overhanging ends produced from (iv); and vi. ligating the filled in end of the chromatin fragments from (v);

b. a single-step process comprising: i. in a single-step, fragmenting the chromatin present in the cells by contacting the chromatin with two restriction enzymes, filling in one or more overhanging ends of the chromatin fragments, and ligating two or more filled in ends;

c. a parallel process comprising: i. fragmenting the chromatin present in the cell with two restriction enzymes in the same or parallel reactions; ii. filling in the overhanging ends from (i), wherein the optional parallel reaction are optionally combined; and iii. ligating two or more filled ends from (ii), wherein the optional parallel reaction are optionally combined;

d. a first MNase process comprising: i. fragmenting the chromatin using micrococcal nuclease (MNase); ii. repairing one or more overhanging ends produced in (i); filling in one or more repaired overhanging ends from (ii); iv. ligating two or more filled ends from (iii);

e. a second MNase process comprising: i. fragmenting the chromatin present in the cells with MNase; ii. in a single step, repairing one or more ends of the chromatin fragments from (i), filling in one or more repaired overhanging ends from (ii), and ligating two or more filled ends from (i); or

f. a third MNase process comprising: i. in a single step, fragmenting the chromatin present in the cells with MNase, repairing one or more ends of the chromatin fragments, filling in one or more repaired overhanging ends, and ligating two or more filled ends.

In certain embodiments, short-read sequencing technologies are used to determine the sequence at the one or more junctions of the one or more end joined nucleic acid fragments. In certain embodiments, long-read sequencing technologies are used to determine the sequence at the one or more junctions of the one or more end joined nucleic acid fragments.

In certain embodiments, the method further comprises assembling a whole genome or partial genome from the determined sequence information. In certain embodiments, the genome is assembled de novo. In certain embodiments, the method further comprises assembling a fully phased diploid whole genome, partial phased genome, phased variant, or individual haplotype from the determined sequence information. In certain embodiments, sequence variants are assigned to single chromosomes. In certain embodiments, the method of phasing different haplotypes comprises calculating the frequency of contact between loci containing particular variants, wherein the frequency of contact between two variants indicates if two variants are on the same molecule. In certain embodiments, the variants are phased, and wherein phasing is determined, at least in part, based on the relative orientation with which a given variant forms contacts with other sequences in the set. In certain embodiments, the orientation is inner, outer, left, or right. In certain embodiments, the frequency of contact between two variants is compared to an expected model to determine whether the two variants are on a same molecule. In certain embodiments, the frequency of contact between two variants is compared to an expected model to determine whether the two variants are on sister chromatids. In certain embodiments, the expected model is determined based on a contact matrix derived from a DNA proximity ligation assay. In certain embodiments, the analysis is performed in an iterative fashion, and wherein data from DNA proximity ligation experiments is used to go from one possible phasing of a variant set to another possible phasing of a variant set. In certain embodiments, analysis of the data from the DNA proximity ligation experiments is performed using gradient descent, hill-climbing, a genetic algorithm, reducing to an instance of the Boolean satisfiability problem (SAT) and solving, or using any combinatorial optimization algorithm. In certain embodiments, the variants to be phased are derived from a single organism or multiple organisms. In certain embodiments, the multiple organisms are from the same species or a different species.

In certain embodiments, the cells and/or cell nuclei are not subjected to mechanical lysis. In certain embodiments, the sample is not subjected to RNA degradation. In certain embodiments, the sample is not contacted with an exonuclease for removal of biotin from unligated ends. In certain embodiments, the sample is not subjected to phenol/chloroform extraction. In certain embodiments, fragmenting the nucleic acid present in the one or more cells comprises enzymatic digestion with an endonuclease that leaves 5′ overhanging ends.

In certain embodiments, the chemical crosslinker comprises an aldehyde. In certain embodiments, the aldehyde comprises formaldehyde. In certain embodiments, reversing the crosslinking comprises contacting the sample with Proteinase K at elevated temperature.

In certain embodiments, the labeled nucleotide is isolated with a specific binding agent that specifically binds to the label. In certain embodiments, the nucleotide is labeled with biotin. In certain embodiments, the specific binding agent comprises avidin and/or streptavidin. In certain embodiments, the specific binding agent is attached to a solid surface.

In certain embodiments, the method further comprises attaching sequencing adapters to the ends of the end joined nucleic acid fragments. In certain embodiments, the method further comprises treating the sample with one or more agents prior to performing a PCR amplification step.

In certain embodiments, the sample is treated with bisulfate or another chemical reagent that preserves DNA methylation information.

In certain embodiments, the cells are cell cycle synchronized. In certain embodiments, the cells in the sample are synchronized in metaphase. In certain embodiments, the sample comprises cells obtained from a diseased tissue. In certain embodiments, the sample comprises cells obtained from a primary tissue. In certain embodiments, the primary tissue is blood.

In certain embodiments, the sample is treated with an agent that isolates all end joined nucleic acids containing a specific nucleic acid sequence. In certain embodiments, the agent is a probe that specifically binds a specific nucleic acid sequence in the one or more junctions. In certain embodiments, the specific nucleic acid sequence is at least 120 base pairs long. In certain embodiments, the specific nucleic acid sequence is within at least 80 base pairs of a restriction site. In certain embodiments, the specific nucleotide sequence has less than 10 repetitive bases. In certain embodiments, the specific nucleic acid sequence has a GC content of between 25% and 80%. In certain embodiments, the probe is labeled. In certain embodiments, the probe is radiolabeled, fluorescently-labeled, biotin-labeled, enzymatically-labeled, or chemically-labeled. In certain embodiments, the probe is a RNA probe, a DNA probe, a locked nucleic acid (LNA) probe, a peptide nucleic acid (PNA) probe, or a hybrid RNA-DNA probe.

In certain embodiments, the method further comprises inferring or determining the three-dimensional structure of a genome comprising determining the sequence of the one or more junctions of the one or more end joined nucleic acid sequences and assembling the three-dimensional structure from the determined sequence information. In certain embodiments, the method further comprises mapping protein-DNA interactions, chromatin post-translational modifications, or RNA-DNA interactions on the three-dimensional structure of the genome. In certain embodiments, protein DNA protein-DNA interactions and/or chromatin post-translational modifications are determined by chromatin immunoprecipitation sequencing (ChIP-seq). In certain embodiments, the method further comprises simultaneous mapping of DNA methylation on the three-dimensional structure. In certain embodiments, the method further comprises distinguishing between heterozygous and homozygous structural variations in samples based at least in part on the determined sequence information. In certain embodiments, the method further comprises resolving the structural variation based at least in part on the determined sequence information. In certain embodiments, the structural variation resolved is a copy number variation.

In another aspect, the present invention provides for a method of mapping complex genomic rearrangements comprising the method of any embodiment herein. In certain embodiments, the complex genomic rearrangements are the result of chromothripsis. In certain embodiments, the method comprises determining one or more breakpoints in the genomic sequence. In certain embodiments, the method further comprises generating an end-to-end structure of a rearranged chromosome. In another aspect, the present invention provides for a method of diagnosing cancer comprising a method as in any embodiment herein.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 is an exemplary flow diagram of exemplary methods disclosed herein. The flow diagram is for illustrative purposes only and it is envisioned that the method disclosed herein can have more or fewer steps than shown in the diagram.

FIG. 2 is a schematic that demonstrates that the disclosed methods can be used to assemble genomes de novo.

FIG. 3—In situ Hi-C was used to map over 15 billion chromatin contacts across nine cell types in human and mouse, achieving 1 kilobase resolution in human lymphoblastoid cells. (A) During in situ Hi-C, DNA-DNA proximity ligation is performed in intact nuclei. (B) Contact matrices from chromosome 14: the whole chromosome, at 500 Kb resolution (top); 86-96 Mb/50 Kb resolution (middle); 94-95 Mb/5 Kb resolution (bottom). Left: GM12878, primary experiment; Right: replicate. The 1D regions corresponding to a contact matrix are indicated in the diagrams above and at left. The intensity of each pixel represents the normalized number of contacts between a pair of loci. Maximum intensity is indicated in the lower left of each panel. (C) Is a comparison in situ HI-C++ generated map of chromosome 7 in GM12878 (last column) to earlier Hi-C maps: Lieberman-Aiden et al., Science 326, 289-293, 2009; Kalhor et al., Nature biotechnology 30, 90-98, 2012, and Jin et al. (D) Mean contacts per pixel vs distance, at various resolutions, compared to published Hi-C experiments (dashed line=10).

FIG. 4—The genome is partitioned into domains that segregate into nuclear subcompartments, corresponding to different patterns of histone modifications. (A) Thousands of domain are annotated (left, black highlight) using the arrowhead transformation (right), which converts domains into arrowhead-shaped motifs (example in yellow). (B) Pearson correlation matrices of the histone mark signal between pairs of loci inside, and within 100 Kb of, a domain. Left: H3K36me3; Right: H3K27me3. (C) Conserved domains on chromosome 3 in GM12878 (left) and IMR90 (right). In GM12878, the highlighted domain (gray) is enriched for H3K27me3 and depleted for H3K36me3. In IMR90, the situation is reversed. Marks at flanking domains are the same in both: the domain to the left is enriched for H3K36me3 and the domain to the right is enriched for H3K27me3. The flanking domains have long-range contact patterns which differ from one another and are preserved in both cell types. In IMR90, the central domain is marked by H3K36me3 and its long-range contact pattern matches the similarly-marked domain on the left. In GM12878, it is decorated with H3K27me3, and the long-range pattern switches, matching the similarly-marked domain to the right. Diagonal submatrices, 10 Kb resolution; long-range interaction matrices, 50 Kb resolution. (D) Each of the six long-range contact patterns Applicants observe exhibits a distinct epigenetic profile. All epigenetic data is from ENCODE experiments in GM12878 except nuclear lamin (derived from skin fibroblast cells) and NAD (HeLa). See Table S8. Each subcompartment also has a visually distinctive contact pattern. (E) Each example shows part of the long-range contact patterns for several nearby genomic intervals lying in different compartments. (F) A large contiguous region on chromosome 19 contains intervals in subcompartments A1, B1, B2, and B4.

FIG. 5—The inventors identified thousands of chromatin loops genome-wide using a local background model. (A) The inventors identified peaks by detecting pixels that are enriched with respect to four local neighborhoods (blowout): horizontal (blue), vertical (green), lower-left (yellow), and donut (black). These “peak” pixels are marked with blue circles (radius=20 Kb) in the lower-left of each heatmap. The number of raw contacts at each peak is indicated. Left: primary GM12878 map; Right: replicate; annotations are completely independent. All contact matrices in these figures are 10 Kb resolution unless noted. (B) Overlap between replicates. (C) (Top) Location of 3D-FISH probes (Bottom) Example cell. (D) APA plot shows the aggregate signal from the 9948 GM12878 loops was made by summing submatrices surrounding each peak in a low-resolution GM12878Hi-C map due to Kalhor et al., Nature biotechnology 30, 90-98, 2012.

FIG. 6—Loops are often preserved across cell types and from human to mouse. (A) Examples of peak and domain preservation across cell types. Annotated peaks are circled in blue. All annotations are completely independent. (B) Of the 3331 loops Applicants annotate in mouse CH12-LX, 1649 (50%) are orthologous to loops in human GM12878. (C-E) Conservation of three-dimensional structure in synteny blocks.

FIG. 7—Loops between promoters and enhancers are strongly associated with gene activation. (A) Histogram showing loop count at promoters (left); restricted to loops where the distal peak locus contains an enhancer (right). (B) Genes whose promoters participate in a loop in GM12878 but not in a second cell type are frequently upregulated in GM12878, and vice-versa. (C) Left: a loop in GM12878, with one anchor at the SELL promoter and the other at a distal enhancer. The gene is on. Right: The loop is absent in IMR90, where the gene is off (D) Left: Two loops in GM12878 are anchored at the promoter of the inactive ADAMTS1 gene. Right: A series of loops and domains appear, along with evident transitive looping. ADAMTS1 is on.

FIG. 8—Many loops demarcate domains; the vast majority of loops are anchored at a pair of convergent CTCF/RAD21/SMC3 binding sites. (A) Histograms of corner score for peak pixels vs. random pixels with an identical distance distribution. (B) Contact matrix for chr4:20.55 Mb-22.55 Mb in GM12878, showing examples of transitive and intransitive looping behavior. (C) % of peak loci bound vs. fold enrichment for 76 DNA-binding proteins. (D) The pairs of CTCF motifs that anchor a loop are nearly all found in the convergent orientation. (E) A peak on chromosome 1 and corresponding ChIP-Seq tracks. Both peak loci contain a single site bound by CTCF, RAD21, and SMC3. The CTCF motifs at the anchors exhibit a convergent orientation.

FIG. 9—Diploid Hi-C maps reveal superdomains and superloops anchored at CTCF-binding repeats on the inactive X chromosome. (A) The frequency of mismatch (maternal-paternal) in SNP allele assignment vs distance between two paired read alignments. Intrachromosomal read pairs are overwhelmingly intramolecular. (B) Preferential interactions between homologs. Left/top is maternal; right/bottom is paternal. The aberrant contact frequency between 6p and 11p (circle) reveals a translocation. (C) Top: In Applicants' unphased Hi-C map of GM12878, the inventors observed two loops joining both the promoter of the maternally-expressed H19 and the promoter of the paternally-expressed Igf2 to a distal locus, HIDAD. Using diploid Hi-C maps, the inventors phase these loops: the HIDAD-H19 loop is present only on the maternal homolog (left) and the HDAD-Igf2 loop is present only on the paternal homolog (right). (D) The inactive (paternal) copy of chromosome X (bottom) is partitioned into two massive “superdomains” not seen in the active (maternal) copy (top). DXZ4 lies at the boundary. (E) The “superloop” between FIRRE and DXZ4 is present in the GM12878 haploid map (top), in the paternal GM12878 map (middle right), and in the map of the female cell line IMR90 (bottom right); it is absent from the maternal GM12878 map (middle left) and the map of the male HUVEC cell line (bottom left).

FIG. 10 is an exemplary flow diagram of embodiments of methods disclosed herein. The flow diagram is for illustrative purposes only, and it is envisioned that the method disclosed herein can have more or fewer steps than shown in the diagram.

FIG. 11 is an exemplary flow diagram of embodiments of methods disclosed herein. The flow diagram is for illustrative purposes only, and it is envisioned that the method disclosed herein can have more or fewer steps than shown in the diagram.

FIG. 12 is an exemplary flow diagram of embodiments of methods disclosed herein. The flow diagram is for illustrative purposes only, and it is envisioned that the method disclosed herein can have more or fewer steps than shown in the diagram.

FIG. 13 is an exemplary flow diagram of embodiments of methods disclosed herein. The flow diagram is for illustrative purposes only and it is envisioned that the method disclosed herein can have more or fewer steps than shown in the diagram.

FIG. 14 is an exemplary flow diagram of embodiments of methods disclosed herein. The flow diagram is for illustrative purposes only, and it is envisioned that the method disclosed herein can have more or fewer steps than shown in the diagram.

FIG. 15 shows an exemplary map generated using Hi-C of three mouse chromosomes including chromothripsis between chr11 and chr13.

FIGS. 16A-16F can demonstrate that Hi-C can be used to ascertain the complete end-to-end structure of a chromothriptic chromosome using a relatively small amount of Hi-C data.

FIG. 17 can demonstrate that the genome-wide Hi-C map of ATDC5 chondrocytes shows unusual interchromosomal signals.

FIG. 18 can demonstrate that ATDC5 chromosomes 11 and 13 (but not 12) show multiple rearrangements.

FIG. 19 can demonstrate a procedure for reconstruction of complex genomic rearrangements.

FIGS. 20-21 can demonstrate complete end-to-end reconstruction of chromosomes “thriven” (20 fragments) and “eleventeen” (55 fragments).

FIG. 22 can demonstrate that chromosomes “thriven” and “eleventeen” appear in SKY data.

FIG. 23 shows a heat map of a human sample generated with Hi-C.

FIG. 24 shows visual language used for phasing.

FIG. 25 can show further attributes of an embodiment of a phaser module.

FIGS. 26-27 can demonstrate that phaser generated chromosome-length phasing blocks with Hi-C data (FIG. 26) that agreed with pedigree data (FIG. 27).

FIGS. 28-31 can demonstrate that the phaser can take in other data types in addition to Hi-C(FIG. 28), generate chromosome and do error correction as needed, JBAT style (FIGS. 29-31).

FIGS. 32-34 show phasing results from PGP1.

FIG. 35 can demonstrate that the phaser can be used with 345× and 80× data.

FIG. 36 shows a graph of the average number of connections v. % in largest component.

FIG. 37 shows a schematic of the principle of a personalized genome.

FIGS. 38-42 shows a flow diagrams representing a personalized genome pipeline (FIG. 38) and aspects thereof (FIGS. 39-42).

FIG. 43 shows a graph that demonstrate the theoretical optimum of phaser approaches.

FIGS. 44-46 can demonstrate the use of deep learning for genome wide analysis of Hi-C and other DNA proximity assay maps.

FIG. 47A-47B—FIG. 47A: Here, the Applicants show contact matrices generated by aligning a Hi-C data set to both the draft Aedes aegypti assembly that the Applicants used as input (left) and the final Aedes aegypti assembly generated by the 3D DNA assembler (right). The center portion of this figure depicts how the 3D DNA assembler uses Hi-C data to split, order and orientate contigs or scaffolds in a draft assembly in order to produce a final assembly. (Dudchenko et al. 2017). FIG. 47B: Here, the Applicants show contact matrices generated by aligning a Hi-C data set to both the draft C. violaceum assembly that the Applicants used as input (left) and the final C. violaceum assembly generated by the 3D DNA assembler (middle). The dotplot (right) is a similarity matrix comparing the de novo C. violaceum Hi-C assembly to the C. violaceum reference assembly. The concordance between the Hi-C assembly and the references, indicated as a continuous line in the dot plots below, indicates that the Applicants correctly assembled the C. violaceum genome.

FIG. 48—Hi-C was applied directly to 5 mL of urine and the Hi-C heatmap of produced from this preliminary experiment showed that chromosome territories were intact. These finding supports the use of urine as a sample type for the purposes of human genome assembly.

FIG. 49A-49B—FIG. 49A: Here the Applicants show contact matrices generated by aligning a Hi-C data set to both the draft P. fluorescens assembly that the Applicants used as input (left) and the final P. fluorescens assembly generated by the 3D DNA assembler (right). FIG. 49B: The dotplot is a similarity matrix comparing the de novo P. fluorescens Hi-C assembly to the P. fluorescens reference assembly. To assess the correctness of their assembly, the Applicants compared the assembly against the reference assembly. The concordance between the Hi-C assembly and the references, indicated as a continuous line in the dot plots below, indicates that the Applicants correctly assembled a majority of the P. fluorescens genome.

FIG. 50—The Applicants have shown that combining Oxford Nanopore based draft assemblies with Illumina-based Hi-C data can be used to reassemble the human genome with a high degree of accuracy and completeness. As a proof of concept, the Applicant chose to sequence DNA and Hi-C ligation products isolated from the Rhodospirillum rubrum bacterium for genome assembly purposes. Preliminary data analysis for these experiments are shown below.

FIG. 51—The workflow disclosed in the present invention that can generate Hi-C and DNA-Seq libraries for Illumina Sequencing, which normally takes 5 days, in less than 6 hours.

FIG. 52—De novo assembly of a lab grown human pathogen, Pseudomonas fluorescens, done in less than 24 hours using the 6 Hour Hi-C/DNA-Seq workflow.

FIG. 53—De novo assembly of a patient derived human pathogen, Klebsiella pneumoniae subsp. Pneumoniae, using 6 Hour Hi-C data generated using Illumina and DNA-Seq data using Oxford Nanopore.

FIG. 54—Hi-C is a whole genome sequencing (WGS) assay that can be used to call SNPs.

FIG. 55—40×Hi-C reads are often enough to phase the majority of SNPs into chromosome-length haploblocks. Chromosome #5 phasing SNP list from 250M PE150 Hi-C reads clinical data, male sample.

FIG. 56—The coverage requirement can be dramatically reduced when Hi-C is paired with long, link-read or population data.

FIG. 57—Juicebox JBAT extension for phasing applications (Robinson, et al., Juicebox.js Provides a Cloud-Based Visualization System for Hi-C Data. Cell Systems 6(2), 2018).

FIG. 58—Hi-C can generate de novo diploid genome assembly using short or short+long reads.

FIG. 59A-59B—Intact Hi-C vs in situ Hi-C. FIG. 59A. Table showing difference between Intact Hi-C and In situ Hi-C. FIG. 59B. Contact maps showing that Intact Hi-C and In situ Hi-C identify the same loops.

FIG. 60A-60D—Intact Hi-C discovers more loops. FIG. 60A. Intact Hi-C contact map with 5 kb resolution. FIGS. 60B, 60C, 60D. Intact Hi-C contact maps with 1 kb resolution. Observed (lower-left): GM12878 intact HiC (9.5B). Control (upper-right): GM12878 in situ HiC 9.2B (Rao 2014).

FIG. 61A-61B—Intact Hi-C discovers more loops. FIG. 61A. Graphs showing the number of loops and loop size for GM12878 using Intact Hi-C and In situ Hi-C. FIG. 61B. Plot showing enrichment of indicated proteins or chromatin modifications at new and old loop anchors.

FIG. 62—APA plots show that SDS weakens the loop signal.

FIG. 63—APA plots show that SDS weakens the loop signal with DNase.

FIG. 64—APA plots show that heating in the presence of SDS weakens the loop signal.

FIG. 65—Intact Hi-C vs in situ Hi-C. APA plots show that intact Hi-C achieves base pair resolution and in situ Hi-C achieves 1 kilobase pair resolution. Top two rows, intact Hi-C; bottom two rows, in situ Hi-C. APAs are centered on CTCF motifs on both axes.

FIG. 66—APA plots showing adjustment of resolution after normalization.

FIG. 67A-67C—Graphs showing counts for contacts around unique CTCF motifs from RH2014. FIG. 67A. Localizations at loop anchors from RH2014 with a uniquely identified responsible CTCF motif and having a single high-resolution (@10 bp res) localization. FIG. 67B. Localizations at loop anchors from RH2014 with a uniquely identified responsible CTCF motif and having at least one high-resolution (@10 bp res) localization.

FIG. 67C. Unique CTCF motifs normalized for motif orientation.

FIG. 68—APA plot showing localizations in relation to the center of a convergent CTCF motif pair. Heatmap of localization density relative to the motif pair is shown. Motif orientations are indicated.

FIG. 69—Intact Hi-C produces true 2D localizations. Graphs showing localizations at the reverse and forward motifs at B.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2^{nd} ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to U.S. patent application Ser. Nos. 15/532,353, 15/753,318, 16/308,386, 16/247,502, and 16/753,718; and International Patent Applications PCT/US2015/063272, PCT/US2016/047644, PCT/US2017/036649, PCT/US2018/054476, and PCT/US2020/033436.

All publications, published patent documents, and patent applications cited herein are hereby incorporated by reference to the same extent as though each individual publication, published patent document, or patent application was specifically and individually indicated as being incorporated by reference.

### OVERVIEW

A major goal in modern biology is defining the interactions between different biological actors in vivo. Over the past few decades, major advances have been made in developing methods to identify the molecular interactions with any given protein. With nucleic acids and in particular genomic DNA it is difficult to determine the interactions in a cell in part because of enormity, at the sequence level, of genomic DNA in a cell. It is believed that genomic DNA adopts a fractal globule state in which the DNA organized in three dimensions such that functionally related genomic elements, for example enhancers and their target genes, are directly interacting or are located in very close spatial proximity. Such close physical proximity between such elements is further believed to play a role in genome biology both in normal development and homeostasis and in disease. During the cell cycle the particular proximity relationships change, further complicating the study of genome dynamics. Understanding, and perhaps controlling, these tertiary interactions at the nucleic acid level has enormous potential to further our understating of the complexities cellular dynamics and perhaps fostering the development of new classes of therapeutics. Thus, methods are needed to investigate these interactions. This disclosure meets those needs.

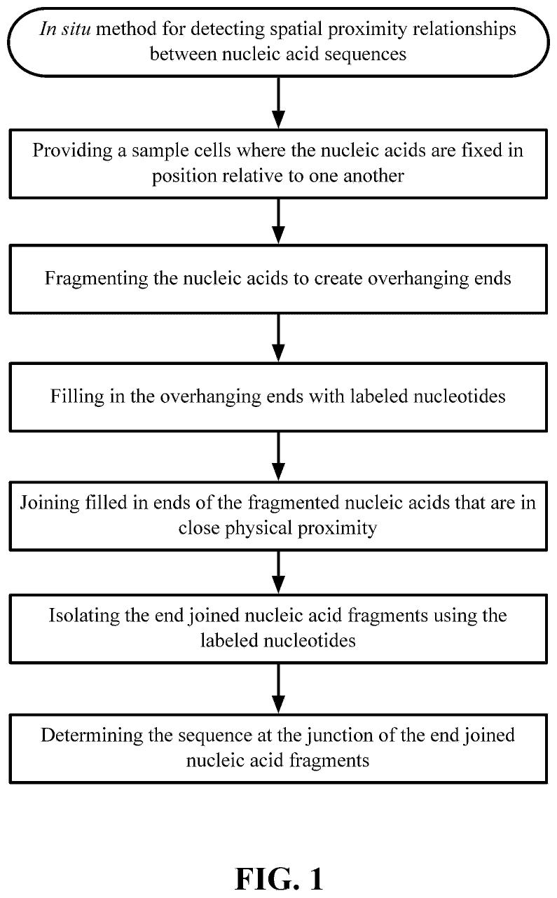

Embodiments disclosed herein provide methods for detecting spatial proximity relationships between DNA in situ. By combining DNA-DNA proximity ligation with high throughput sequencing in order to measure how frequently positions in the human genome come into close physical proximity, the disclosed method can simultaneously map substantially all of the interactions of DNAs in a cell, including spatial arrangements of DNA. A flowchart depicting a non-limiting example of the methods disclosed is given in FIG. 1. Some of the advantages of the disclosed method are that is can be completed on a small sample of cells, without dilution of the sample. This lack of dilution yields many more contacts than previous methods used to define DNA/DNA interactions, such as chromosome Conformation Capture (3C) and Hi-C technology (see, e.g., Dekker et al., Science 295:1306-1311 (2002) and Lieberman-Aiden et al., Science 326:289-93 (2009)).

Embodiments disclosed herein also provide methods for detecting spatial proximity relationships between genomic DNA in a cell with up to base pair resolution (Intact Hi-C, described further herein). Previous methods could, at best, reach kilobase pair resolution (see, e.g., Rao S S, Huntley M H, Durand N C, et al. A 3D map of the human genome at kilobase resolution reveals principles of chromatin looping [published correction appears in Cell. 2015 Jul. 30; 162(3):687-8]. Cell. 2014; 159(7):1665-1680). In certain embodiments, cells are crosslinked to preserve the spatial proximity of proteins and nucleic acids within the cell; and nuclei are isolated from the cells. A key element of the present invention is performing the following steps in situ (i.e., the steps are performed inside permeabilized nuclei): fragmenting of genomic DNA and ligating genomic DNA fragments that are in contact or close proximity. In certain embodiments, the base pair resolution is achieved by using conditions that maintain protein complex integrity in the nuclei of the crosslinked cells (i.e., the proteins are not denatured). Applicants unexpectedly discovered that SDS treatment, to permeabilize the nuclei, destabilizes the spatial proximity of the fragmented genomic DNA in the crosslinked nuclei. In certain embodiments, the nuclei are permeabilized under conditions that preserve cohesin complex integrity in the crosslinked nuclei. In certain embodiments, the base pair resolution is further achieved by sequencing the fragments using a method that sequences across the ligation junction (ligation junction sequencing).

Embodiments disclosed herein also provide methods for identifying chromatin loops that could not be identified by previous methods due to the decreased resolution. Thus, previously unidentified CTCF dependent and CTCF independent loops can now be identified. Moreover, exact sequences can be identified where a loop anchor is present. Additionally, the exact sequences that are bound by proteins can be identified (CTCF or CTCF independent) that can arrest cohesin at a loop anchor. This is consistent with data showing that deletion of CTCF does not eliminate all loops, but deletion of cohesin does eliminate all loops (see, e.g., Rao S S P, Huang S C, Glenn St Hilaire B, et al. Cohesin Loss Eliminates All Loop Domains. Cell. 2017; 171(2):305-320.e24).

Genes are located at a particular position on a particular chromosome, but the elements that regulate their activity can lie far away. Understanding these distal regulatory sequences is essential to understanding how genes turn on and off in a healthy person, and how this process goes awry in disease. But finding distal regulatory sequences has been an open problem for over 30 years. In certain embodiments, promoter/enhancer loops can be identified, in particular in cases where many regulatory sequences are present in close proximity to a gene. Thus, a method having high resolution is required to determine specific enhancers or regulatory sequences in the enhancers. In certain embodiments, an enhancer can be assigned to a gene based on the loops identified. In certain embodiments, loops may change under certain conditions and an enhancer can be assigned to more than one gene. In certain embodiments, enhancer/promoter loops can be identified that are present under only certain conditions.

In certain embodiments, the spatial information provided by the methods herein can be combined with already available data for motif predictions, transcription factor binding motifs, epigenetic state of the sequences (e.g., ChIP-seq, bisulfite sequencing), and binding of proteins at the sequences (e.g., ChIP-seq) (see, e.g., ENCODE Consortium; ENCODE Project Consortium. An integrated encyclopedia of DNA elements in the human genome. Nature. 2012; 489(7414):57-74; and Davis C A, Hitz B C, Sloan C A, et al. The Encyclopedia of DNA elements (ENCODE): data portal update. Nucleic Acids Res. 2018; 46(D1):D794-D801). For example, the histone modification of a specific sequence in a loop anchor can indicate the conditions at the sequence required for loop formation. Histone modifications may also recruit specific chromatin binding proteins and these can be mapped to a specific sequence based on chromatin immunoprecipitation (ChIP) data. The presence of a specific motif may indicate that cohesin is being arrested by a specific protein (e.g., a transcription factor). As used herein transcription factor refers to any factor recruited to a gene or regulatory sequence, such as, a general transcription factor, activator, repressor, coactivator, nuclear receptor, chromatin remodeling factor, etc.

Using the three-dimensional genome sequencing approach disclosed herein, it is now possible to comprehensively identify all distal regulators of all genes in a sample population of cells. The information available, will make it possible to assess the impact of candidate drugs on specific cellular circuits, hastening the process of drug discovery and for biological research in general. The information available will also enable the mapping of genomic structural and sequence variations.

The methods described herein also allow for determining the whole genome sequence of a cell simultaneously with detecting spatial proximity relationships between genomic DNA. Applicants discovered that the sequencing reads obtained for the joined fragments cover approximately the same percentage of the genome as conventional whole genome sequencing. Thus, in certain embodiments, sequence variants (e.g., SNPs) can be identified in addition to the spatial data.

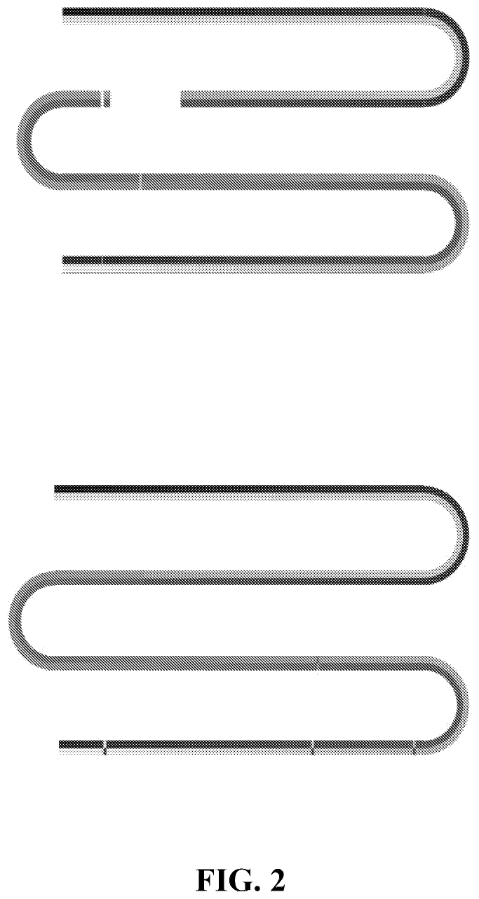

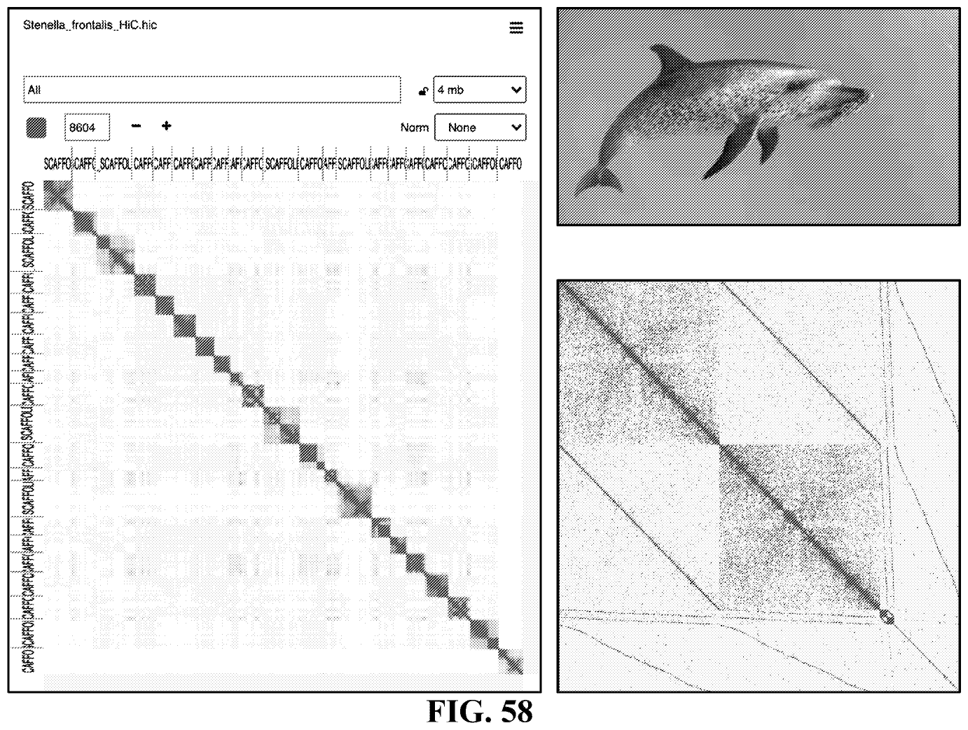

In certain embodiments, the spatial proximity data can be used to assemble a genome haplotype. One of the other major advances enabled by the methods disclosed herein is de novo genome assembly. As shown in FIG. 2 and FIG. 58, the combination of the disclosed methods and high through put sequencing can be used to assemble genomes de novo. The image at top of FIG. 2 represents the correct assembly of human chromosome 20. At bottom is shown a de novo assembly of human chromosome 20 from 100 kb fragments, created using data generated with the methods disclosed herein. With the exception of a few small inversions, the assembly is perfect. The maps allow the creation of de novo genome assemblies without the use of mate pair reads.

Embodiments disclosed herein also provide methods for phasing different haplotypes. In certain embodiments, the methods described herein can provide suitable data suitable for phasing different haplotypes. In some embodiments, the sequence information determined by the disclosed methods may be used to phase polymorphisms and/or assemble individual haplotypes, and distinguish between heterozygous and homozygous structural variations. Thus, sequence variants can be assigned to a specific chromosome.

In certain embodiments, the sequence information determined by the disclosed methods may be used to resolve genomic structural genomic variation, including copy number variations, estimate the 1D distance between two fragments of DNA from the same chromosome, assess syntenic relationships between two or more organisms at arbitrary resolution, and/or generate phylogenetic trees and/or ancestral genomes.

In certain embodiments, sequence variants associated with a phenotype can be assigned to a specific chromosome or haplotype and can be assigned to a specific gene based on enhancer/promoter contacts (see, e.g., Welter, D. et al. The NHGRI GWAS catalogue, a curated resource of SNP-trait associations. Nucleic Acids Res. 42, D1001-D1006 (2014); Wood, A. R. et al. Defining the role of common variation in the genomic and biological architecture of adult human height. Nat. Genet. 46, 1173-1186 (2014); Ripke, S. et al. Biological insights from 108 schizophrenia-associated genetic loci. Nature 511, 421-427 (2014); Okbay, A. et al. Genome-wide association study identifies 74 loci associated with educational attainment. Nature 533, 539-542 (2016); Sudlow, C. et al. UK biobank: an open access resource for identifying the causes of a wide range of complex diseases of middle and old age. PLoS Med. 12, 1-10 (2015); Bycroft et al., The UK Biobank resource with deep phenotyping and genomic data. Nature 562, 203-209 (2018); and 1000 Genomes Project Consortium. A global reference for human genetic variation. Molecular cell, 526(7571):68-74, 2015). Moreover, variants present in a loop may be assigned to a gene. The variants may be present in an enhancer and enhancers may be assigned to specific genes. Thus, the present invention provides for linking variants to genes to phenotypes (e.g., disease). Previous studies showed that disease-associated variants are enriched in specific regulatory chromatin states (see, e.g., Ernst, J. et al. Mapping and analysis of chromatin state dynamics in nine human cell types. Nature 473, 43-49 (2011)), evolutionarily conserved elements (Lindblad-Toh, K. et al. A high-resolution map of human evolutionary constraint using 29 mammals. Nature 478, 476-482 (2011)), histone marks (Trynka, G. et al. Chromatin marks identify critical cell types for fine mapping complex trait variants. Nature Genet. 45, 124-130 (2013)) and accessible regions (Maurano, M. T. et al. Systematic localization of common disease-associated variation in regulatory DNA. Science 337, 1190-1195 (2012)), thus showing the importance of assigning variants in regulatory sequences to the correct chromosomes and genes.

The methods described herein can also be used for mapping highly complex genomic rearrangements, such as those that occur during chromothripsis. Chromothripsis is a shattering and haphazard recombination of one or several chromosomes (see e.g. Stephens et al. Cell 144:27 (2011). Chromothripsis is believed to result in oncogenic mutations and thus cancer. Approximately three percent of tested cancer cases contain at least one chromothriptic chromosome. Methods for detecting and end-to-end reconstruction of such complex rearrangements are further discussed in Example 6. In some embodiments, these methods can be used to screen for, diagnose, prognose, treat, and/or prevent a disease. In some embodiments, the disease can be a cancer.

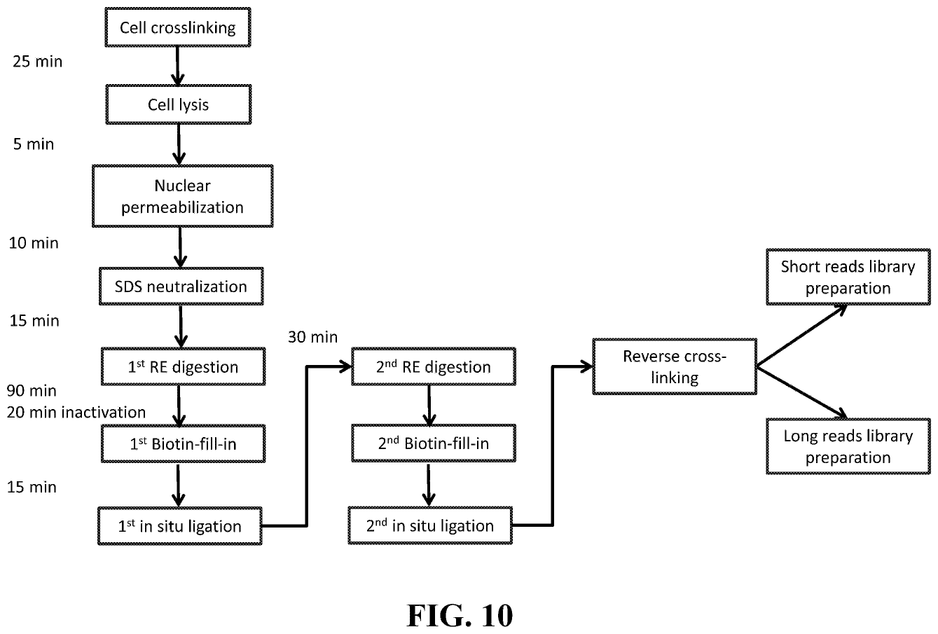

Embodiments disclosed herein also provide methods that reduce the cost and time required to determine spatial proximity relationships between genomic DNA in a cell (e.g., for Intact Hi-C and in situ Hi-C). In certain embodiments, the methods can be performed in approximately 5-10 hours (e.g., 7 hours). Previous methods required about 3 days. These methods can be combined with any embodiment herein, for example, methods that produce base pair resolution. Moreover, the Hi-C methods exemplified in this specification can be modified as is shown in FIGS. 10-14. These embodiments can have further advantages over these other methods such as faster turn-around and workflow, increased chromatin accessibility, increased chromatin accessibility that facilitates mapping of fine interactions with fewer reads than currently available methods, among others that will be appreciated in view of this disclosure.

Specific examples of information that can be obtained from the disclosed methods and the analysis of the results thereof, include without limitation uni- or multiplex, 3 dimensional genome mapping, genome assembly, one dimensional genome mapping, the use of single nucleotide polymorphisms to phase genome maps, for example to determine the patterns of chromosome inactivation, such as for analysis of genomic imprinting, the use of specific junctions to determine karyotypes, including but not limited to chromosome number alterations (such as unisomies, uniparental disomies, and trisomies), translocations, inversions, duplications, deletions and other chromosomal rearrangements, the use of specific junctions correlated with disease to aid in diagnosis.

### In Situ Methods for Detecting Spatial Nucleic Acid Proximity

Disclosed herein are in situ methods for detecting spatial proximity relationships between nucleic acid sequences in a sample, such as DNA sequences, for example in a cell or multiple cells. The methods include providing a sample of one or more cells, nuclear extract, cellular milieu or system of nucleic acids of interest that include nucleic acids. In some embodiments, the spatial relationships in the cell is locked in, for example cross-linked or otherwise stabilized. For example, a sample of cells can be treated with a cross-linker to lock in the spatial information or relationship about the molecules in the cells, such as the DNA in the cell. The nucleic acids present are fragmented in situ to yield nucleic acids with overhanging ends (e.g., 5′ overhanging end) or ends in need of repair depending on how the DNA is fragmented. The overhanging ends are then filled in and/or repaired in situ, for example using a DNA polymerase, such as available from a commercial source. The filled in or repaired nucleic acid fragments are thus blunt ended at the end filled 5′ end. The fragments are then end joined in situ at the filled in or repaired end, for example, by ligation using a commercially available nucleic acid ligase, or otherwise attached to another fragment that is in close physical proximity. The ligation, or other attachment procedure, for example nick translation or strand displacement, creates one or more end joined nucleic acid fragments having a junction, for example a ligation junction, wherein the site of the junction, or at least within a few bases, includes one or more labeled nucleic acids, for example, one or more fragmented nucleic acids that have had their overhanging ends filled and joined together. While this step typically involves a ligase, it is contemplated that any means of joining the fragments can be used, for example any chemical or enzymatic means. Further, it is not necessary that the ends be joined in a typical 3′-5′ ligation.

In certain embodiments, to identify the created ligation junction a labeled nucleotide is used. In one example embodiment, one or more labeled nucleotides are incorporated into the ligated junction. For example, the overhanging or repaired ends may be filled in using a DNA polymerase that incorporates one or more labeled nucleotides during the filling in or repairing step described above.

In some embodiments, the nucleic acids are cross-linked, either directly, or indirectly, and the information about spatial relationships between the different DNA fragments in the cell, or cells, is maintained during the joining step, and substantially all of the end joined nucleic acid fragments formed at this step were in spatial proximity in the cell prior to the crosslinking step. Previously it was believed that the crosslinking locked in the spatial proximity of DNA sequences in the cell. However, Applicants disclose herein that denaturing conditions can still cause part of the spatial information to be lost by denaturing crosslinked protein complexes necessary to hold the DNA in a locked position. Once the DNA ends are joined the information about which sequences were in spatial proximity to other sequences in the cell is locked into the end joined fragments. It has been found that in some situations, it is not necessary to hold the nucleic acids in place using a chemical fixative or crosslinking agent. Thus, in some embodiments, no crosslinking agent is used. In still other embodiments, the nucleic acids are held in position relative to each other by the application of non-crosslinking means, such as by using agar or other polymer to hold the nucleic acids in position.

The labeled nucleotide present in the junction is used to isolate the one or more end joined nucleic acid fragments using a binding agent specific to the labeled nucleotide. The sequence is determined at the junction of the one or more end joined nucleic acid fragments, thereby detecting spatial proximity relationships between nucleic acid sequences in a cell. In some embodiments, such as for genome assembly, essentially all of the sequence of the end joined fragments is determined. In some embodiments, determining the sequence of the junction of the one or more end joined nucleic acid fragments includes nucleic acid sequencing. In some embodiments, determining the sequence of the junction of the one or more end joined nucleic acid fragments includes using a probe that specifically hybridizes to the nucleic acid sequences both 5′ and 3′ of the junction of the one or more end joined nucleic acid fragments, for example using an RNA probe, a DNA probe, a locked nucleic acid (LNA) probe, a peptide nucleic acid (PNA) probe, or a hybrid RNA-DNA probe. In exemplary embodiments of the disclosed method, the location is determined or identified for nucleic acid sequences both 5′ and 3′ of the ligation junction of the one or more end joined nucleic acid fragments relative to source genome and/or chromosome. In some embodiments, the junction identified is correlated with a disease state. In some embodiments, the junction identified is correlated with an environmental condition. In some embodiments, the sequenced end joined fragments are assembled to create an assembled genome or portion thereof, such as a chromosome or sub-fraction thereof. In some embodiments, information from one or more ligation junctions derived from a sample consisting of a mixture of cells from different organisms, such as mixture of microbes, is used to identify the organisms present in the sample and their relative proportions. In some example, the sample is derived from patient samples.

The disclosed methods are also particularly suited to monitoring disease states, such as disease state in an organism, for example a plant or an animal subject, such as a mammalian subject, for example a human subject. Certain disease states may be caused and/or characterized by the differential formation of certain target joins. For example, certain interactions may occur in a diseased cell but not in a normal cell. In other examples, certain interactions may occur in a normal cell but not in diseased cell. Thus, using the disclosed methods a profile of the interaction between DNA sequences in vivo, can be correlated with a disease state. The target join profile correlated with a disease can be used as a “fingerprint” to identify and/or diagnose a disease in a cell, by virtue of having a similar “fingerprint.” In addition, the profile can be used to monitor a disease state, for example to monitor the response to a therapy, disease progression and/or make treatment decisions for subjects.

The ability to obtain an interaction profile allows for the diagnosis of a disease state, for example by comparison of the profile present in a sample with the correlated with a specific disease state, wherein a similarity in profile indicates a particular disease state.

Accordingly, aspects of the disclosed methods relate to diagnosing a disease state based on target junction profile correlated with a disease state, for example cancer, or an infection, such as a viral or bacterial infection. It is understood that a diagnosis of a disease state could be made for any organism, including without limitation plants, and animals, such as humans.

Aspects of the present disclosure relate to the correlation of an environmental stress or state with a target junction profile, such as a sample of cells, for example a culture of cells, can be exposed to an environmental stress, such as but not limited to heat shock, osmolarity, hypoxia, cold, oxidative stress, radiation, starvation, a chemical (for example a therapeutic agent or potential therapeutic agent) and the like. After the stress is applied, a representative sample can be subjected to analysis, for example at various time points, and compared to a control, such as a sample from an organism or cell, for example a cell from an organism, or a standard value.

In some embodiments, the disclosed methods can be used to screen chemical libraries for agents that modulate DNA interaction profiles, for example that alter the interaction profile from an abnormal one, for example correlated to a disease state to one indicative of a disease free state. By exposing cells, or fractions thereof, tissues, or even whole animals, to different members of the chemical libraries, and performing the methods described herein, different members of a chemical library can be screened for their effect on interaction profiles simultaneously in a relatively short amount of time, for example using a high throughput method.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks. As used herein the term “test agent” refers to any agent that that is tested for its effects, for example its effects on a cell. In some embodiments, a test agent is a chemical compound, such as a chemotherapeutic agent, antibiotic, or even an agent with unknown biological properties.

Appropriate agents can be contained in libraries, for example, synthetic or natural compounds in a combinatorial library. Numerous libraries are commercially available or can be readily produced; means for random and directed synthesis of a wide variety of organic compounds and biomolecules, including expression of randomized oligonucleotides, such as antisense oligonucleotides and oligopeptides, also are known. Alternatively, libraries of natural compounds in the form of bacterial, fungal, plant and animal extracts are available or can be readily produced. Additionally, natural or synthetically produced libraries and compounds are readily modified through conventional chemical, physical and biochemical means, and may be used to produce combinatorial libraries. Such libraries are useful for the screening of a large number of different compounds.

The compounds identified using the methods disclosed herein can serve as conventional “lead compounds” or can themselves be used as potential or actual therapeutics. In some instances, pools of candidate agents can be identified and further screened to determine which individual or sub-pools of agents in the collective have a desired activity.

Appropriate samples for use in the methods disclosed herein include any conventional biological sample obtained from an organism or a part thereof, such as a plant, animal, and the like. In particular embodiments, the biological sample is obtained from an animal subject, such as a human subject. A biological sample is any solid or fluid sample obtained from, excreted by or secreted by any living organism, including without limitation, single celled organisms, such as yeast, protozoans, and amoebas among others, multicellular organisms (such as plants or animals, including samples from a healthy or apparently healthy human subject or a human patient affected by a condition or disease to be diagnosed or investigated, such as cancer). For example, a biological sample can be a biological fluid obtained from, for example, blood, plasma, serum, urine, bile, ascites, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease, such as a rheumatoid arthritis, osteoarthritis, gout or septic arthritis). A sample can also be a sample obtained from any organ or tissue (including a biopsy or autopsy specimen, such as a tumor biopsy) or can include a cell (whether a primary cell or cultured cell) or medium conditioned by any cell, tissue or organ. Exemplary samples include, without limitation, cells, cell lysates, blood smears, cyto-centrifuge preparations, cytology smears, bodily fluids (e.g., blood, plasma, serum, saliva, sputum, urine, bronchoalveolar lavage, semen, etc.), tissue biopsies (e.g., tumor biopsies), fine-needle aspirates, and/or tissue sections (e.g., cryostat tissue sections and/or paraffin-embedded tissue sections). In other examples, the sample includes circulating tumor cells (which can be identified by cell surface markers). In particular examples, samples are used directly (e.g., fresh or frozen), or can be manipulated prior to use, for example, by fixation (e.g., using formalin) and/or embedding in wax (such as formalin-fixed paraffin-embedded (FFPE) tissue samples). It will be appreciated that any method of obtaining tissue from a subject can be utilized, and that the selection of the method used will depend upon various factors such as the type of tissue, age of the subject, or procedures available to the practitioner. Standard techniques for acquisition of such samples are available. See, for example Schluger et al., J. Exp. Med. 176:1327-33 (1992); Bigby et al., Am. Rev. Respir. Dis. 133:515-18 (1986); Kovacs et al., NEJM 318:589-93 (1988); and Ognibene et al., Am. Rev. Respir. Dis. 129:929-32 (1984).

### Crosslinking

In some embodiments of the disclosed method the nucleic acids present in the cell or cells are fixed in position relative to each other by chemical crosslinking, for example by contacting the cells with one or more chemical cross linkers. This treatment locks in the spatial relationships between portions of nucleic acids in a cell. Any method of fixing the nucleic acids in their positions can be used. In some embodiments, the cells are fixed, for example with a fixative, such as an aldehyde, for example formaldehyde or gluteraldehyde. In some embodiments, a sample of one or more cells is cross-linked with a cross-linker to maintain the spatial relationships in the cell. For example, a sample of cells can be treated with a cross-linker to lock in the spatial information or relationship about the molecules in the cells, such as the DNA and RNA in the cell. In other embodiments, the relative positions of the nucleic acid can be maintained without using crosslinking agents. For example, the nucleic acids can be stabilized using spermine and spermidine (see Cullen et al., Science 261, 203 (1993), which is specifically incorporated herein by reference in its entirety). Other methods of maintaining the positional relationships of nucleic acids are known in the art. In some embodiments, nuclei are stabilized by embedding in a polymer such as agarose. In some embodiments, the cross-linker is a reversible cross-linker. In some embodiments, the cross-linker is reversed, for example after the fragments are joined and the spatial information is locked in. In specific examples, the nucleic acids are released from the cross-linked three-dimensional matrix by treatment with an agent, such as a proteinase, that degrade the proteinaceous material from the sample, thereby releasing the end ligated nucleic acids for further analysis, such as determination of the nucleic acid sequence. In specific embodiments, the sample is contacted with a proteinase, such as Proteinase K. In some embodiments of the disclosed methods, the cells are contacted with a crosslinking agent to provide the cross-linked cells. In some examples, the cells are contacted with a protein-nucleic acid crosslinking agent, a nucleic acid-nucleic acid crosslinking agent, a protein-protein crosslinking agent or any combination thereof. By this method, the nucleic acids present in the sample become resistant to special rearrangement and the spatial information about the relative locations of nucleic acids in the cell is maintained. In certain embodiments, the cells are cross linked such that the cohesin complex is not denatured. In some examples, a cross-linker is a reversible, such that the cross-linked molecules can be easily separated in subsequent steps of the method. In some examples, a cross-linker is a non-reversible cross-linker, such that the cross-linked molecules cannot be easily separated. In some examples, a cross-linker is light, such as UV light. In some examples, a cross linker is light activated. These cross-linkers include formaldehyde, disuccinimidyl glutarate, UV light, psoralens and their derivatives such as aminomethyltrioxsalen, glutaraldehyde, ethylene glycol bis[succinimidylsuccinate], bissulfosuccinimidyl suberate, 1-Ethyl-3-[3-dimethylaminopropyl]carbodiimide (EDC) bis[sulfosuccinimidyl] suberate (BS³) and other compounds known to those skilled in the art, including those described in the Thermo Scientific Pierce Crosslinking Technical Handbook, Thermo Scientific (2009) as available on the world wide web at piercenet. com/files/1601673_Crosslink_HB_Intl.pdf.

As used herein the term “contacting” refers to Placement in direct physical association, including both in solid or liquid form, for example contacting a sample with a crosslinking agent or a probe. As used herein the term “Crosslinking agent” refers to a chemical agent or even light, which facilitates the attachment of one molecule to another molecule. Crosslinking agents can be protein-nucleic acid crosslinking agents, nucleic acid-nucleic acid crosslinking agents, and protein-protein crosslinking agents. Examples of such agents are known in the art. In some embodiments, a crosslinking agent is a reversible crosslinking agent. In some embodiments, a crosslinking agent is a non-reversible crosslinking agent.

### Isolated Nuclei

In some embodiments, the cells are lysed to release the cellular contents, for example after crosslinking. In some examples the nuclei are lysed as well, while in other examples, the nuclei are maintained intact, which can then be isolated and optionally lysed, for example using a reagent that selectively targets the nuclei or other separation technique known in the art. In some examples, the sample is a sample of permeabilized nuclei, multiple nuclei, or isolated nuclei. In certain embodiments the cells are synchronized cells, (such at various points in the cell cycle, for example metaphase) before nuclei are isolated.

### Permeabilizing Nuclei

In certain examples, the methods include permeabilizing nuclei. In certain embodiments, nuclei of the present invention can be permeabilized according to any method known in the art. In some cases, the nuclei may be permeabilized to allow access for nucleic acid processing reagents. The permeabilization may be performed in a way to minimally perturb the spatial proximity of nucleic acids, protein folding, organelles, and/or nuclei. In certain embodiments, the nuclei are permeabilized, such that protein complexes do not fall apart or proteins are not denatured. In some instances, the cells may be permeabilized using a permeabilization agent. Examples of permeabilization agents include NP40, digitonin, tween, streptolysin, exonuclease 1 buffer (NEB) and pepsin, and cationic lipids. In other instances, the cells, organelles, and/or nuclei may be permeabilized using hypotonic shock and/or ultrasonication. In other cases, the nucleic acid processing reagents e.g., enzymes such as nuclease, polymerase and/or ligase, may be highly charged, which may allow them to permeabilize through the membranes of the nuclei. Other embodiments include use of cell penetrating peptides to deliver cargo to the nuclei and allow capture of material. In certain embodiments, permeabilization steps, including pre-permeabilization are automated.

In certain embodiments, nuclei are permeabilized with a detergent. In certain embodiments, the detergent is non-ionic. In certain embodiments, the concentration of the detergent is sufficient to permeabilize the nuclei without denaturing proteins in the nuclei. In certain embodiments, NP40, digitonin, or tween is used. For example, the concentration of detergent used herein may be from 0.005% to 1%, from 0.01% to 0.8%, from 0.01% to 0.6%, from 0.01% to 0.4%, from 0.01% to 0.2%, from 0.01% to 0.1%, from 0.005% to 0.05%, from 0.01% to 0.03%, from 0.015% to 0.025%, from 0.018% to 0.022%, from 0.015% to 0.017%, from 0.016% to 0.018%, from 0.017% to 0.019%, from 0.018% to 0.02%, from 0.019% to 0.021%, from 0.02% to 0.022%, or from 0.021% to 0.023%. In some cases, the concentration of the detergent may be about 0.01%, about 0.015%, about 0.02%, about 0.025%, or about 0.03%. For example, the concentration of the detergent may be about 0.02%. In certain embodiments, SDS is used at concentrations below 0.5%, such as 0.1, 0.05, or less than 0.01%. In certain embodiments, the nuclei are not heated during permeabilization.

### Fragmenting, End-Repair, Fill-In and Ligation

In some embodiments, in order to create discrete portions of nucleic acid that can be joined together in subsequent steps of the methods, the nucleic acids present in the cells, such as cross-linked cells, are fragmented. The fragmentation can be done by a variety of methods, such as enzymatic and chemical cleavage. For example, DNA can be fragmented using an endonuclease that cuts a specific sequence of DNA and leaves behind a DNA fragment with a 5′ overhang, thereby yielding fragmented DNA. In other examples, an endonuclease can be selected that cuts the DNA at random spots and yields overhangs or blunt ends. In some embodiments, fragmenting the nucleic acid present in the one or more cells comprises enzymatic digestion with an endonuclease that leaves 5′ overhanging ends. Enzymes that fragment, or cut, nucleic acids and yield an overhanging sequence are known in the art and can be obtained from such commercial sources as New England BioLabs® and Promega®. One of ordinary skill in the art can choose the restriction enzyme without undue experimentation. One of ordinary skill in the art will appreciate that using different fragmentation techniques, such as different enzymes with different sequence requirements, will yield different fragmentation patterns and therefore different nucleic acid ends. The process of fragmenting the sample can yield ends that are capable of being joined.

In certain embodiments, the ends of the fragmented DNA is repaired (e.g., end repair). Commercial reagents and protocols are available for DNA end repair. Fragmentation of polynucleotide molecules may result in fragments with a heterogeneous mix of blunt and 3′- and 5′-overhanging ends. It is therefore desirable to repair the fragment ends using methods or kits known in the art to generate ends that are optimal for ligation, for example, blunt sites of chromatin fragments. In a particular embodiment, the fragment ends of the nucleic acids are blunt ended. One method of the invention involves repairing the fragment ends with nucleotide triphosphates and a nucleic acid polymerase. The nucleotide triphosphates may contain a labeling modification, for example biotin or similar protein binding ligand, that allows selection of the end repaired fragments. The polymerase may be Klenow DNA polymerase or similar nucleic acid polymerase, that may have exonuclease activity in order to remove any 3′ overhanging ends. The reaction may be carried out with all four nucleotides, of which 0-4 may carry labeling modifications. The reaction may be carried out with a single labelled nucleoside triphosphate, and three unlabeled triphosphates, or may be carried out with two, three or four labeled nucleotides.

As used herein the term “Nucleic acid (molecule or sequence)” refers to a deoxyribonucleotide or ribonucleotide polymer including without limitation, cDNA, mRNA, genomic DNA, and synthetic (such as chemically synthesized) DNA or RNA or hybrids thereof. The nucleic acid can be double-stranded (ds) or single-stranded (ss). Where single-stranded, the nucleic acid can be the sense strand or the antisense strand. Nucleic acids can include natural nucleotides (such as A, T/U, C, and G), and can also include analogs of natural nucleotides, such as labeled nucleotides. Some examples of nucleic acids include the probes disclosed herein.

The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U). Nucleotides include those nucleotides containing modified bases, modified sugar moieties, and modified phosphate backbones, for example as described in U.S. Pat. No. 5,866,336 to Nazarenko et al.

Examples of modified base moieties which can be used to modify nucleotides at any position on its structure include, but are not limited to: 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N˜6-sopentenyladenine, 1-methylguanine, 1-methylinosine, 2,2-dimethylguanine, 2-methyladenine, 2-methylguanine, 3-methylcytosine, 5-methyl cytosine, N6-adenine, 7-methylguanine, 5-methylaminomethyluracil, methoxyarninomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-S-oxyacetic acid, 5-methyl-2-thiouracil, 3-(3-amino-3-N-2-carboxypropyl) uracil, 2,6-diaminopurine and biotinylated analogs, amongst others.

Examples of modified sugar moieties which may be used to modify nucleotides at any position on its structure include, but are not limited to arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

Ligation may be carried out in situ using any ligase known in the art and described further in the examples to obtain covalently linked joined DNA molecules. The ligation reaction may be carried out using any suitable ligase, for example, T3 or T4 ligase. Covalently linked: Refers to a covalent linkage between atoms by the formation of a covalent bond characterized by the sharing of pairs of electrons between atoms. In one example, a covalent link is a bond between an oxygen and a phosphorous, such as phosphodiester bonds in the backbone of a nucleic acid strand. In another example, a covalent link is one between a nucleic acid protein, another protein and/or nucleic acid that has been crosslinked by chemical means. In another example, a covalent link is one between fragmented nucleic acids.

In some embodiments, the end joined DNA that includes a labeled nucleotide is captured with a specific binding agent that specifically binds a capture moiety, such as biotin, on the labeled nucleotide. In some embodiments, the capture moiety is adsorbed or otherwise captured on a surface. In specific embodiments, the end target joined DNA is labeled with biotin, for instance by incorporation of biotin-14-CTP or other biotinylated nucleotide during the filling in of the 5′ overhang, for example with a DNA polymerase, allowing capture by streptavidin. This step can also be referred to herein as “biotin filling” or “biotin-fill-in”. In some embodiments, the step(s) of biotin filling can be completed in about 1 to about 45 minutes such as about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or about 45 minutes. Any additional biotin filing steps as discussed elsewhere herein, can also be completed in about in about 1 to about 45 minutes such as about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or about 45 minutes.

As used herein the term “biotin-14-CTP” refers to a biologically active analog of cytosine-5′-triphosphate that is readily incorporated into a nucleic acid by polymerase or a reverse transcriptase. In some examples, biotin-14-CTP is incorporated into a nucleic acid fragment that has a 3′ overhang.

As used herein the term “capture moieties” refers to molecules or other substances that when attached to a nucleic acid molecule, such as an end joined nucleic acid, allow for the capture of the nucleic acid molecule through interactions of the capture moiety and something that the capture moiety binds to, such as a particular surface and/or molecule, such as a specific binding molecule that is capable of specifically binding to the capture moiety.

Other means for labeling, capturing, and detecting nucleic acid probes include: incorporation of aminoallyl-labeled nucleotides, incorporation of sulfhydryl-labeled nucleotides, incorporation of allyl- or azide-containing nucleotides, and many other methods described in Bioconjugate Techniques (2^{nd} Ed), Greg T. Hermanson, Elsevier (2008), which is specifically incorporated herein by reference. In some embodiments the specific binding agent has been immobilized for example on a solid support, thereby isolating the target nucleic molecule of interest. By “solid support or carrier” is intended any support capable of binding a targeting nucleic acid. Well-known supports or carriers include glass, polystyrene, polypropylene, polyethylene, dextran, nylon, amylases, natural and modified celluloses, polyacrylamides, agarose, gabbros and magnetite. The nature of the carrier can be either soluble to some extent or insoluble for the purposes of the present disclosure. The support material may have virtually any possible structural configuration so long as the coupled molecule is capable of binding to targeting probe. Thus, the support configuration may be spherical, as in a bead, or cylindrical, as in the inside surface of a test tube, or the external surface of a rod. Alternatively, the surface may be flat such as a sheet or test strip. After capture, these end joined nucleic acid fragments are available for further analysis, for example to determine the sequences that contributed to the information encoded by the ligation junction, which can be used to determine which DNA sequences are close in spatial proximity in the cell, for example to map the three dimensional structure of DNA in a cell such as genomic and/or chromatin bound DNA. In some embodiments, the sequence is determined by PCR, hybridization of a probe and/or sequencing, for example by sequencing using high-throughput paired end sequencing. In some embodiments determining the sequence at the one or more junctions of the one or more end joined nucleic acid fragments comprises nucleic acid sequencing, such as short-read sequencing technologies or long-read sequencing technologies. In some embodiments, nucleic acid sequencing is used to determine two or more junctions within an end-joined concatemer simultaneously.

As used herein the term “specific binding agent” refers to an agent that binds substantially or preferentially only to a defined target such as a protein, enzyme, polysaccharide, oligonucleotide, DNA, RNA, recombinant vector or a small molecule. In an example, a “specific binding agent that specifically binds to the label” is capable of binding to a label that is covalently linked to a targeting probe.

In some embodiments, determining the sequence of a junction includes using a probe that specifically binds to the junction at the site of the two joined nucleic acid fragments. In particular embodiments, the probe specifically hybridizes to the junction both 5′ and 3′ of the site of the join and spans the site of the join. A probe that specifically binds to the junction at the site of the join can be selected based on known interactions, for example in a diagnostic setting where the presence of a particular target junction, or set of target junctions, has been correlated with a particular disease or condition. It is further contemplated that once a target junction is known, a probe for that target junction can be synthesized.

In some embodiments, the end joined nucleic acids are selectively amplified. In some examples, to selectively amplify the end joined nucleic acids, a 3′ DNA adaptor and a 5′ RNA, or conversely a 5′ DNA adaptor and a 3′ RNA adaptor can be ligated to the ends of the molecules can be used to mark the end joined nucleic acids. Using primers specific for these adaptors only end joined nucleic acids will be amplified during an amplification procedure such as PCR. In some embodiments, the target end joined nucleic acid is amplified using primers that specifically hybridize to the adaptor nucleic acid sequences present at the 3′ and 5′ ends of the end joined nucleic acids. In some embodiments, the non-ligated ends of the nucleic acids are end repaired. In some embodiments attaching sequencing adapters to the ends of the end ligated nucleic acid fragments.

As used herein the term “primers” refers to short nucleic acid molecules, such as a DNA oligonucleotide, which can be annealed to a complementary target nucleic acid molecule by nucleic acid hybridization to form a hybrid between the primer and the target nucleic acid strand. A primer can be extended along the target nucleic acid molecule by a polymerase enzyme. Therefore, primers can be used to amplify a target nucleic acid molecule, wherein the sequence of the primer is specific for the target nucleic acid molecule, for example so that the primer will hybridize to the target nucleic acid molecule under very high stringency hybridization conditions.

The specificity of a primer increases with its length. Thus, for example, a primer that includes 30 consecutive nucleotides will anneal to a target sequence with a higher specificity than a corresponding primer of only 15 nucleotides. Thus, to obtain greater specificity, probes and primers can be selected that include at least 5, 10, 15, 20, 25, 30, 35, 40, 45, 50 or more consecutive nucleotides.

In particular examples, a primer is at least 15 nucleotides in length, such as at least 5 contiguous nucleotides complementary to a target nucleic acid molecule. Particular lengths of primers that can be used to practice the methods of the present disclosure include primers having at least 5, at least 10, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, or more contiguous nucleotides complementary to the target nucleic acid molecule to be amplified, such as a primer of 5-60 nucleotides, 15-50 nucleotides, 15-30 nucleotides or greater.

Primer pairs can be used for amplification of a nucleic acid sequence, for example, by PCR, or other nucleic-acid amplification methods known in the art. An “upstream” or “forward” primer is a primer 5′ to a reference point on a nucleic acid sequence. A “downstream” or “reverse” primer is a primer 3′ to a reference point on a nucleic acid sequence. In general, at least one forward and one reverse primer are included in an amplification reaction. PCR primer pairs can be derived from a known sequence, for example, by using computer programs intended for that purpose such as Primer (Version 0.5, © 1991, Whitehead Institute for Biomedical Research, Cambridge, Mass.).

Methods for preparing and using primers are described in, for example, Sambrook et al. (1989) Molecular Cloning: A Laboratory Manual, Cold Spring Harbor, N.Y.; Ausubel et al. (1987) Current Protocols in Molecular Biology, Greene Publ. Assoc. & Wiley-Intersciences.

In some embodiments, such as those exemplified by FIG. 10, the method can include a second digestion, biotin fill-in, and ligation. The second ligation can be followed by reverse cross linking and library construction. FIG. 10 shows flow chart of an embodiment of a method for detecting spatial nucleic acid proximity that can begin with cell cross linking. Methods and techniques of cell cross crosslinking suitable for use with this and other embodiments are described in greater detail elsewhere herein. After a period of time (e.g. about 25 minutes) the cells can be lysed. Methods and techniques of lysing cells suitable for use with this and other embodiments are described in greater detail elsewhere herein. After a period of time to allow for lysis to occur (e.g. about 5 minutes, the nucleus can be permeabilized using a suitable method. Methods and techniques of permeabilizing a cell nucleus suitable for use with this and other embodiments are described in greater detail elsewhere herein. After neutralization of the chromatin, two serial rounds of restriction enzyme (RE) digestion, biotin-fill-in, and ligation can be completed. The use of two REs used serially to cut the chromatin can increase chromatin accessibility. In each round, biotin-fill-in and in situ ligation can be completed in about 15 min and about 30 min, respectively. This can decrease the turnaround time and speed up work-flow. The second ligation can be followed by revers cross-linking and subsequent library construction.

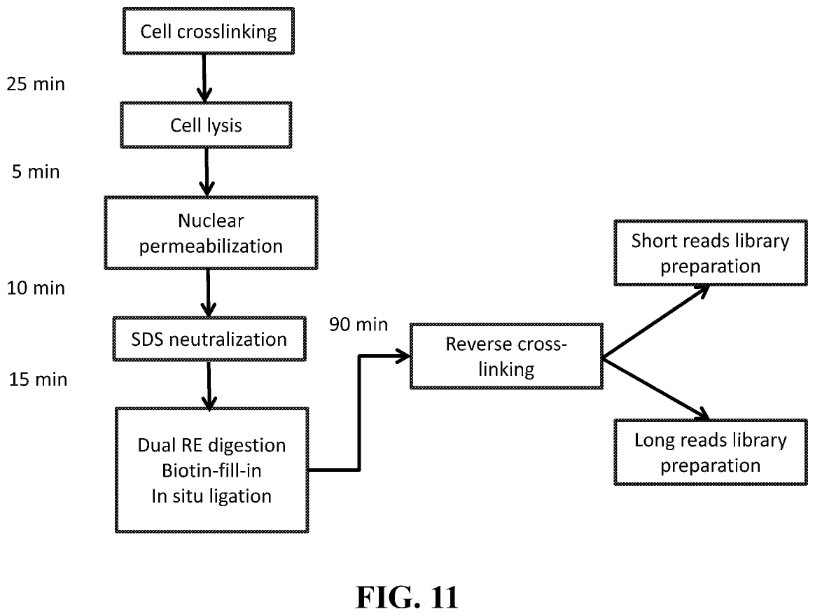

In some embodiments, such as those exemplified by FIG. 11, the method can begin again with cell cross linking, cell lysis, nuclear permeabilization and neutralization. In these embodiments, neutralization can be followed by a dual restriction enzyme digestion and biotin-fill-in that takes place in a single step, which is in contrast to the serial rounds of RE digestion, biotin-fill-in, and ligation of the embodiments exemplified by FIG. 10. The single-step dual RE digestion, biotin fill-in, and ligation can be followed with reverse cross linking and library preparation.

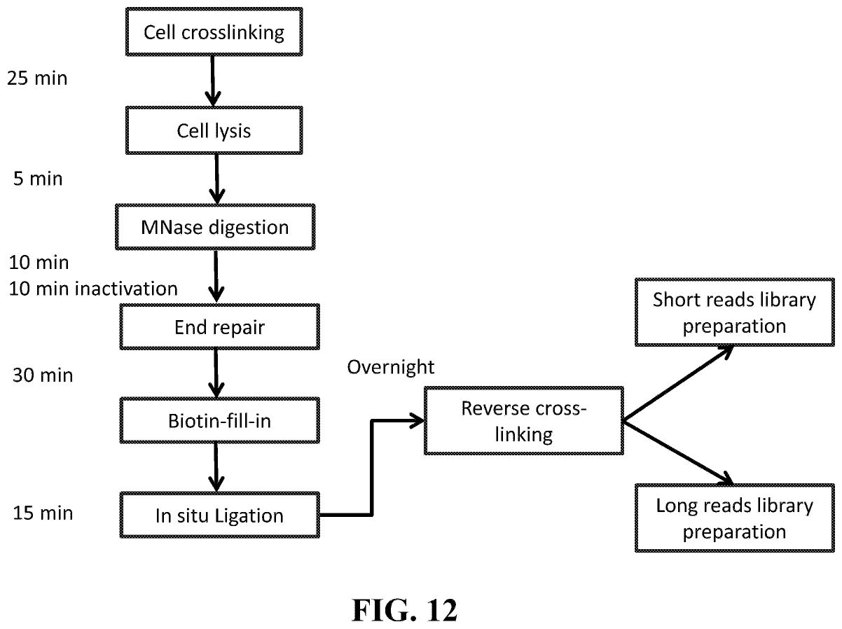

In some embodiments, such as those exemplified by FIG. 12, the method can begin again with cell cross linking and cell lysis. In these embodiments, cell lysis can be followed by micrococcal nuclease (MNase) digestion. The use of MNase digestion here can increase chromatin accessibility and mapping of fine interaction with fewer reads. Ends can be repaired following MNase digestion. The end repair step can rescue modified nucleotide ends to facilitate ligation. End repair can be followed by biotin-fill-in and in situ ligation. In some embodiments, the biotin-fill-in can be completed in about 15 minutes. In some embodiments, ligation can be allowed to go overnight. Ligation can be followed by reverse cross linking and library construction.

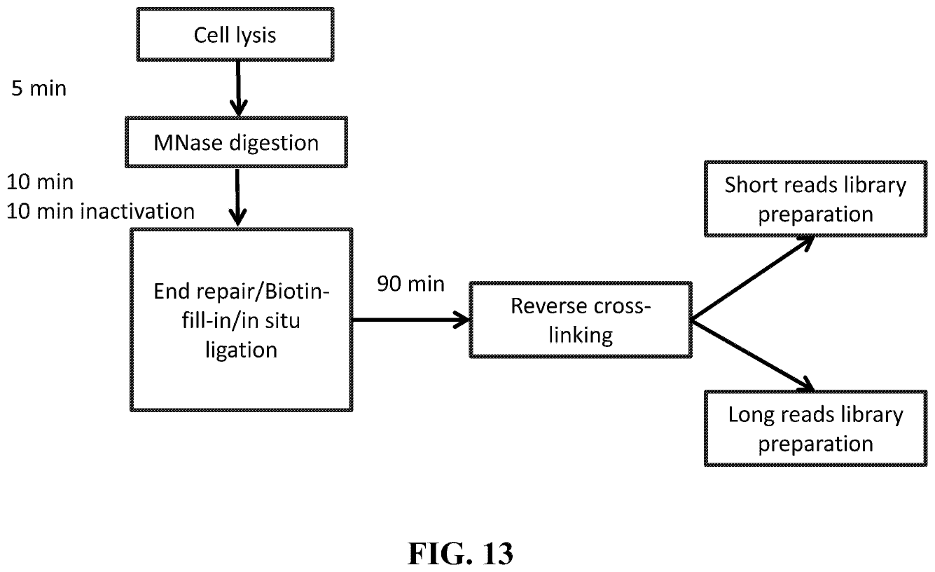

In some embodiments, such as those exemplified by FIG. 13, the method can begin as in FIG. 12 with cell cross-linking (not shown) and cell lysis. In some embodiments, the method can continue with MNase digestion followed by a single-step that includes end-repair, biotin-fill-in, and ligation. In some embodiments, the MNase can be deactivated prior to subsequent reactions that may be performed in the presence of the MNase. Optionally, in some embodiments, after cell lysis the method can continue with a single step that includes MNase digestion, end-repair, biotin-fill-in, and ligation. After ligation products are formed, the method can continue with reverse cross linking and library construction. In some embodiments, the step that includes end-repair, biotin-fill-in, and ligation can be completed in about 90 minutes. After the step that includes ligation, the method can continue with reverse cross linking and library construction. In some embodiments, end-repair, biotin-fill-in, ligation and optionally chromatin digestion can be completed in about 90 to about 110 minutes, which can decrease the turn-around time and increase the rate of work flow. Like the embodiments discussed in relation to FIG. 12, MNase can increase the chromatin accessibility and facilitate mapping of fine interactions with fewer reads.

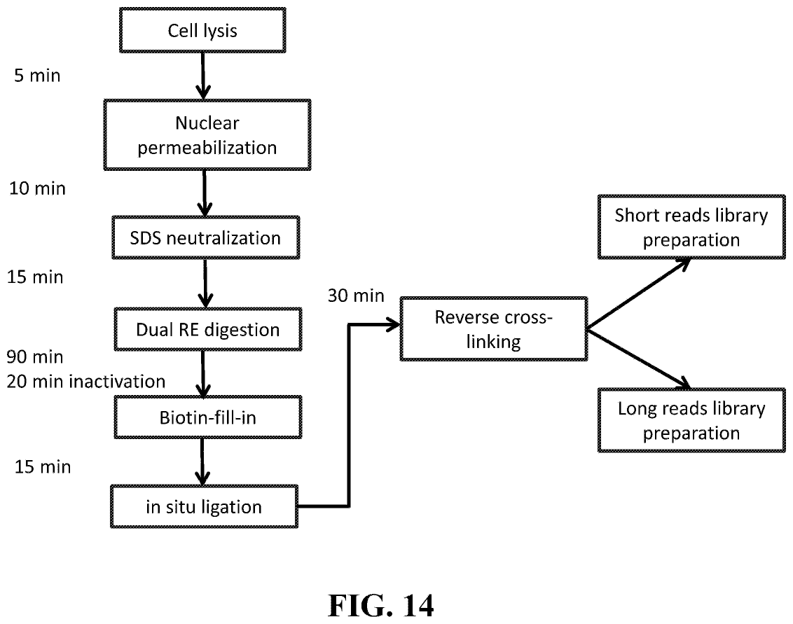

In some embodiments, such as those exemplified by FIG. 14, the method can begin with cell cross-linking (not shown), nuclear permeabilization, and neutralization as previously described elsewhere herein. Following neutralization, a dual parallel RE digest can be performed. This can be followed by biotin-fill-in and ligation in separate steps. In some embodiments, biotin-fill-in and ligation can be completed in about 15 and 30 minutes, respectively. Like other embodiments described herein, ligation can be followed by reverse cross linking and library construction.

As used herein the term “isolated” refers to an “isolated” biological component (such as the end joined fragmented nucleic acids or nuclei as described herein) has been substantially separated or purified away from other biological components in the cell of the organism, in which the component naturally occurs, for example, extra-chromatin DNA and RNA, proteins and organelles. Nucleic acids and proteins that have been “isolated” include nucleic acids and proteins purified by standard purification methods, for example from a sample. The term also embraces nucleic acids and proteins prepared by recombinant expression in a host cell as well as chemically synthesized nucleic acids. It is understood that the term “isolated” does not imply that the biological component is free of trace contamination, and can include nucleic acid molecules that are at least 50% isolated, such as at least 75%, 80%, 90%, 95%, 98%, 99%, or even 100% isolated.

### Sequencing

In certain embodiments, the one or more end joined nucleic acid fragments are sequenced to determine the junction and the sequence of the entire joined fragments. In certain embodiments, ligation junction sequencing is performed to ensure an accurate sequence of the ligation junction is obtained. In certain embodiments, the exact sequences with the highest contacts are determined. In a typical paired end sequencing reaction fragments are approximately 500 base pairs and the fragments are sequenced from each end. Ligation junction sequencing requires shorter fragments and/or sequencing from a single end. In certain embodiments, the nucleic acid fragments for ligation junction sequencing are between about 100 and about 400 bases in length, such as about 100, about 150, about 200, about 250, about 300, about 350, about 400, or about 450 bases in length, for example form about 100 to about 400, about 200 to about 300, about 250 to about 350, and about 250 to about 300 base pairs in length and the like. In specific examples, end joined fragments are selected for sequence determination that are between about 200 and 300 base pairs in length. In certain embodiments, end joined fragments of about 250 base pairs in length are sequenced from both ends. In certain embodiments, end joined fragments of about 300 base pairs in length are sequenced from a single end.

As used herein the term “junction” refers to a site where two nucleic acid fragments or joined, for example using the methods described herein. A junction encodes information about the proximity of the nucleic acid fragments that participate in formation of the junction. For example, junction formation between to nucleic acid fragments indicates that these two nucleic acid sequences where in close proximity when the junction was formed, although they may not be in proximity in linear nucleic acid sequence space. Thus, a junction can define long range interactions. In some embodiments, a junction is labeled, for example with a labeled nucleotide, for example to facilitate isolation of the nucleic acid molecule that includes the junction.

In some embodiments, the nucleic acids present in the ligated sample are purified, for example using ethanol precipitation. In example embodiments of the disclosed method the cell nuclei are not subjected to mechanical lysis. In some example embodiments, the sample is not subjected to RNA degradation. In specific embodiments, the sample is not contacted with an exonuclease to remove biotin from un-ligated ends. In some embodiments, the sample is not subjected to phenol/chloroform extraction.

As used herein the term “DNA sequencing” refers to the process of determining the nucleotide order of a given DNA molecule. In certain embodiments, the sequencing can be performed using automated Sanger sequencing. In certain embodiments, sequencing comprises high-throughput (formerly “next-generation”) technologies to generate sequencing reads from the one or more end joined nucleic acid fragments. In DNA sequencing, a read is an inferred sequence of base pairs (or base pair probabilities) corresponding to all or part of a single DNA fragment. A typical sequencing experiment involves fragmentation of the genome into millions of molecules or generating complementary DNA (cDNA) fragments, which are size-selected and ligated to adapters. The set of fragments is referred to as a sequencing library, which is sequenced to produce a set of reads. Methods for constructing sequencing libraries are known in the art (see, e.g., Head et al., Library construction for next-generation sequencing: Overviews and challenges. Biotechniques. 2014; 56(2): 61-77; Trombetta, J. J., Gennert, D., Lu, D., Satija, R., Shalek, A. K. & Regev, A. Preparation of Single-Cell RNA-Seq Libraries for Next Generation Sequencing. Curr Protoc Mol Biol. 107, 4 22 21-24 22 17, doi:10.1002/0471142727.mb0422s107 (2014). PMCID:4338574). A “library” or “fragment library” may be a collection of nucleic acid molecules derived from one or more nucleic acid samples, in which fragments of nucleic acid have been modified, generally by incorporating terminal adapter sequences comprising one or more primer binding sites and identifiable sequence tags. In certain embodiments, the library members (e.g., genomic DNA; cDNA) may include sequencing adaptors that are compatible with use in, e.g., Illumina's reversible terminator method, long read nanopore sequencing, Roche's pyrosequencing method (454), Life Technologies' sequencing by ligation (the SOLiD platform) or Life Technologies' Ion Torrent platform. Examples of such methods are described in the following references: Margulies et al (Nature 2005 437: 376-80); Schneider and Dekker (Nat Biotechnol. 2012 Apr. 10; 30(4):326-8); Ronaghi et al (Analytical Biochemistry 1996 242: 84-9); Shendure et al (Science 2005 309: 1728-32); Imelfort et al (Brief Bioinform. 2009 10: 609-18); Fox et al (Methods Mol. Biol. 2009; 553:79-108); Appleby et al (Methods Mol. Biol. 2009; 513:19-39); and Morozova et al (Genomics. 2008 92:255-64), which are incorporated by reference for the general descriptions of the methods and the particular steps of the methods, including all starting products, reagents; and final products for each of the steps.

In certain embodiments, sequencing of the isolated end joined nucleic acid fragments results in whole genome sequencing. Whole genome sequencing (also known as WGS, full genome sequencing, complete genome sequencing, or entire genome sequencing) is the process of determining the complete DNA sequence of an organism's genome at a single time. This entails sequencing all of an organism's chromosomal DNA as well as DNA contained in the mitochondria and, for plants, in the chloroplast. “Whole genome amplification” (“WGA”) refers to any amplification method that aims to produce an amplification product that is representative of the genome from which it was amplified. Non-limiting WGA methods include Primer extension PCR (PEP) and improved PEP (I-PEP), Degenerated oligonucleotide primed PCR (DOP-PCR), Ligation-mediated PCR (LMP), T7-based linear amplification of DNA (TLAD), and Multiple displacement amplification (MDA).

In certain embodiments, the present invention includes whole exome sequencing by enriching for the one or more end joined nucleic acid fragments representative of the exome (e.g., hybrid selection, HYbrid Capture Hi-C(Hi-C2)). Exome sequencing, also known as whole exome sequencing (WES), is a genomic technique for sequencing all of the protein-coding genes in a genome (known as the exome) (see, e.g., Ng et al., 2009, Nature volume 461, pages 272-276). It consists of two steps: the first step is to select only the subset of DNA that encodes proteins. These regions are known as exons—humans have about 180,000 exons, constituting about 1% of the human genome, or approximately 30 million base pairs. The second step is to sequence the exonic DNA using any high-throughput DNA sequencing technology. In certain embodiments, whole exome sequencing is used to determine somatic mutations in genes associated with disease (e.g., cancer mutations).

In certain embodiments, the present invention includes targeted sequencing by enriching for the one or more end joined nucleic acid fragments representative of a panel of genes or sequences (e.g., hybrid selection, HYbrid Capture Hi-C(Hi-C2), discussed further herein). Targeted gene sequencing panels are useful tools for analyzing specific mutations in a given sample. Focused panels contain a select set of genes or gene regions that have known or suspected associations with the disease or phenotype under study. In certain embodiments, targeted sequencing is used to detect mutations associated with a disease in a subject in need thereof. Targeted sequencing can increase the cost-effectiveness of variant discovery and detection.

In certain embodiments, the present invention includes amplification to increase the number of copies of a nucleic acid molecule, such as one or more end joined nucleic acid fragments that includes a junction, such as a ligation junction. The resulting amplification products are called “amplicons.” Amplification of a nucleic acid molecule (such as a DNA or RNA molecule) refers to use of a technique that increases the number of copies of a nucleic acid molecule (including fragments).

An example of amplification is the polymerase chain reaction (PCR), in which a sample is contacted with a pair of oligonucleotide primers under conditions that allow for the hybridization of the primers to a nucleic acid template in the sample. The primers are extended under suitable conditions, dissociated from the template, re-annealed, extended, and dissociated to amplify the number of copies of the nucleic acid. This cycle can be repeated. The product of amplification can be characterized by such techniques as electrophoresis, restriction endonuclease cleavage patterns, oligonucleotide hybridization or ligation, and/or nucleic acid sequencing.

Other examples of in vitro amplification techniques include quantitative real-time PCR; reverse transcriptase PCR (RT-PCR); real-time PCR (rt PCR); real-time reverse transcriptase PCR (rt RT-PCR); nested PCR; strand displacement amplification (see U.S. Pat. No. 5,744,311); transcription-free isothermal amplification (see U.S. Pat. No. 6,033,881, repair chain reaction amplification (see WO 90/01069); ligase chain reaction amplification (see European patent publication EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134) amongst others.

Furthermore, the methods disclosed herein can readily be combined with other techniques, such as hybrid capture after library generation (to target specific parts of the genome), chromatin immunoprecipitation after ligation (to examine the chromatin environment of regions associated with specific proteins), bisulfate treatment, (to probe the methylation state of DNA). For examples the information from one or more ligation junctions is used to infer and/or determine the three-dimensional structure of the genome. In some embodiments, the information from one or more ligation junctions is used to simultaneously map protein-DNA interactions and DNA-DNA interactions or RNA-DNA interactions and DNA-DNA interactions. In some embodiments, the information from one or more ligation junctions is used to simultaneously map methylation and three-dimensional structure. In some embodiments, the information from more than one ligation junction is used to assemble whole genomes or parts of genomes. In some embodiments, the sample is treated to accentuate interactions between contiguous regions of the genome. In some embodiments, the cells in the sample are synchronized in metaphase.

In one example embodiment, hybrid capture after library generation comprises treating a library of end joined nucleic acid fragments generated using the methods described above with an agent that isolates end joined nucleic acid fragments comprising specific nucleic acid sequence (target sequence). In certain example embodiments, the specific nucleic acid sequence is at least 50, at least 60, at least 70, at least 80, at least 90, at least 100, at least 110, at least 120, at least 130, at least 140, at least 150, at least 160, at least 170, at least 180, at least 190, or at least 200 base pairs long. In certain example embodiments, the specific nucleic acid sequence is within at least 50, at least 60, at least 70, at least, 80, at least 90, or at least 100 base pairs, in either the 5′ or 3′ direction, of a restriction site. In certain example embodiments, the specific nucleic sequence comprises less than ten repetitive bases. In certain other example embodiments, the GC content of the specific nucleic acid sequence is between 25% and 80%, between 40% and 70%, or between 50% and 60%.

In certain example embodiments, the agent that isolates the end joined nucleic acid fragments comprising the specific nucleic acid sequence is a probe. The probe may be labeled. In certain example embodiments, the probe is radiolabeled, fluorescently-labeled, enzymatically-labeled, or chemically labeled. In certain other example embodiments, the probe may be labeled with a capture moiety, such as a biotin-label. When the probe is labeled with a capture moiety, the capture moiety may be used to isolate the end joined nucleic acid fragments using techniques such as those known in the art and described previously. The exact sequence of the isolated end-joined nucleic acid fragments may then be determined, for example, by sequencing as described previously.

### Detection of Junctions by Hybridization

In some embodiments of the disclosed methods, determining the identity of a nucleic acid, such as a target junction, includes detection by nucleic acid hybridization. Nucleic acid hybridization involves providing a probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, PNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization requires fewer mismatches. One of skill in the art will appreciate that hybridization conditions can be designed to provide different degrees of stringency.

As used herein the term “target junction” refers to any nucleic acid present or thought to be present in a sample that the information of a junction between an end joined nucleic acid fragment about which information would like to be obtained, such as its presence or absence.

As used herein the term “complementary” refers to a double-stranded DNA or RNA strand consists of two complementary strands of base pairs. Complementary binding occurs when the base of one nucleic acid molecule forms a hydrogen bond to the base of another nucleic acid molecule. Normally, the base adenine (A) is complementary to thymidine (T) and uracil (U), while cytosine (C) is complementary to guanine (G). For example, the sequence 5′-ATCG-3′ of one ssDNA molecule can bond to 3′-TAGC-5′ of another ssDNA to form a dsDNA. In this example, the sequence 5′-ATCG-3′ is the reverse complement of 3′-TAGC-5′.

Nucleic acid molecules can be complementary to each other even without complete hydrogen-bonding of all bases of each molecule. For example, hybridization with a complementary nucleic acid sequence can occur under conditions of differing stringency in which a complement will bind at some but not all nucleotide positions.

In general, there is a tradeoff between hybridization specificity (stringency) and signal intensity. Thus, in one embodiment, the wash is performed at the highest stringency that produces consistent results and that provides a signal intensity greater than approximately 10% of the background intensity. Thus, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest. In some examples, RNA is detected using Northern blotting or in situ hybridization (Parker & Barnes, Methods in Molecular Biology 106:247-283, 1999); RNAse protection assays (Hod, Biotechniques 13:852-4, 1992); and PCR-based methods, such as reverse transcription polymerase chain reaction (RT-PCR) (Weis et al., Trends in Genetics 8:263-4, 1992).

As used herein the term “binding or stable binding (of an oligonucleotide)” refers to an oligonucleotide, such as a nucleic acid probe that specifically binds to a target junction in an end joined nucleic acid fragment, binds or stably binds to a target nucleic acid if a sufficient amount of the oligonucleotide forms base pairs or is hybridized to its target nucleic acid. For example, depending on the hybridization conditions, there need not be complete matching between the probe and the nucleic acid target, for example there can be mismatch, or a nucleic acid bubble. Binding can be detected by either physical or functional properties.

As used herein the term “binding site” refers to a region on a protein, DNA, or RNA to which other molecules stably bind. In one example, a binding site is the site on an end joined nucleic acid fragment.

As used herein the term “detect” refers to determining if an agent (such as a signal or particular nucleic acid or protein) is present or absent. In some examples, this can further include quantification in a sample, or a fraction of a sample, such as a particular cell or cells within a tissue.

As used herein the term “detectable label” refers to a compound or composition that is conjugated directly or indirectly to another molecule to facilitate detection of that molecule. Specific, non-limiting examples of labels include fluorescent tags, enzymatic linkages, and radioactive isotopes and other physical tags, such as biotin. In some examples, a label is attached to a nucleic acid, such as an end-joined nucleic acid, to facilitate detection and/or isolation of the nucleic acid.

As used herein the term “probe” refers to an isolated nucleic acid capable of hybridizing to a target nucleic acid (such as end joined nucleic acid fragment). A detectable label or reporter molecule can be attached to a probe. Typical labels include radioactive isotopes, enzyme substrates, co-factors, ligands, chemiluminescent or fluorescent agents, haptens, and enzymes.

Methods for labeling and guidance in the choice of labels appropriate for various purposes are discussed, for example, in Sambrook et al., Molecular Cloning: A Laboratory Manual, Cold Spring Harbor Laboratory Press (1989) and Ausubel et al., Current Protocols in Molecular Biology, Greene Publishing Associates and Wiley-Intersciences (1987).

Probes are generally at least 5 nucleotides in length, such as at least 10, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50 at least 51, at least 52, at least 53, at least 54, at least 55, at least 56, at least 57, at least 58, at least 59, at least 60, or more contiguous nucleotides complementary to the target nucleic acid molecule, such as 50-60 nucleotides, 20-50 nucleotides, 20-40 nucleotides, 20-30 nucleotides or greater.

As used herein the term “targeting probe” refers to a probe that includes an isolated nucleic acid capable of hybridizing to a junction in an end joined nucleic acid fragment, wherein the probe specifically hybridizes to the end joined nucleic acid fragment both 5′ and 3′ of the site of the junction and spans the site of the junction.

In one embodiment, the hybridized nucleic acids are detected by detecting one or more labels attached to the sample nucleic acids. The labels can be incorporated by any of a number of methods. In one example, the label is simultaneously incorporated during the amplification step in the preparation of the sample nucleic acids. Thus, for example, polymerase chain reaction (PCR) with labeled primers or labeled nucleotides will provide a labeled amplification product. In one embodiment, transcription amplification, as described above, using a labeled nucleotide (such as fluorescein-labeled UTP and/or CTP) incorporates a label into the transcribed nucleic acids.

Detectable labels suitable for use include any composition detectable by spectroscopic, photochemical, biochemical, immunochemical, electrical, optical or chemical means. Useful labels include biotin for staining with labeled streptavidin conjugate, magnetic beads (for example DYNABEADS™), fluorescent dyes (for example, fluorescein, Texas red, rhodamine, green fluorescent protein, and the like), radiolabels (for example, ³H, ¹²⁵I, ³⁵S, ¹⁴C, or ³²P), enzymes (for example, horseradish peroxidase, alkaline phosphatase and others commonly used in an ELISA), and colorimetric labels such as colloidal gold or colored glass or plastic (for example, polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Means of detecting such labels are also well known. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

The label may be added to the target (sample) nucleic acid(s) prior to, or after, the hybridization. So-called “direct labels” are detectable labels that are directly attached to or incorporated into the target (sample) nucleic acid prior to hybridization. In contrast, so-called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected (see Laboratory Techniques in Biochemistry and Molecular Biology, Vol. 24: Hybridization With Nucleic Acid Probes, P. Tijssen, ed. Elsevier, N.Y., 1993).

### Phasing

In certain embodiments, the methods described herein can provide suitable data suitable for phasing different haplotypes. Thus, also described herein are methods of phasing different haplotypes. In some embodiments, the method can include calculating a frequency of contact between loci containing particular variants, wherein the frequency of contact is determined using sequencing reads derived from a DNA proximity ligation assay (such as any of those described and demonstrated elsewhere herein), wherein the frequency of contact between two variants indicates if two variants are on the same molecule. In certain example embodiments, the frequency of contact between two variants is compared to an expected model to determine whether the two variants are on the same molecule. The expected model may be determined based on a contact matrix derived from a DNA proximity ligation assay, wherein reads are represented as pixels in the contact map and wherein contact frequency is a function of distance from a diagonal of the contact matrix. In certain example embodiments, the analysis may be done in an iterative fashion and wherein in data from DNA proximity ligation experiments is used to go from one possible phasing of a variant set to another possible phasing of a variant set. The analysis of the data from the DNA proximity ligation experiments is performed using gradient descent, hill-climbing, a genetic algorithm, reducing to an instance of the Boolean satisfiability problem (SAT) and solving, or using any combinatorial optimization algorithm.

The methods disclosed herein may also be used to assist in phasing of the human genome. Phasing can be performed de novo and using population data. The 3D contact maps can be used to assess the accuracy of phasing results.

The methods disclosed herein may also be used to analyze karyotype evolution in given group of species as well as to detect karyotype polymorphisms, even at low-coverage. The karyotype data can be used to identify phylogenetic relationships, either by itself or with sequence level data.

The methods disclosed herein may also be used to substitute for inter-species chromosome painting, including at low coverage.

The methods disclosed herein may also be used to estimate the distance along the 1D sequence between any two given genomic sequences.

The methods disclosed herein may use the features of 3D contact maps. For example, identification of chromatin motifs in their proper convergent orientation can be used to properly orient other contigs in the assembly.

The methods disclosed herein can include a phasing module that utilizes a signal produced from a DNA proximity assay such as anyone described herein. The module can take as input a list of variants (.vcf) e.g. generated by realignment of data from a DNA proximity assay described herein (e.g. Hi-C and others) as well as list of dedupped Hi-C alignments (Jucier mind file). Various embodiments can be capable of producing chromosome-length haploblocks solely from ENCODE data. Various embodiments can take advantage of partial phasing data such as long-read phasing, population phasing, etc. An embodiment of such a method is exemplified in Example 7.

### Genome Assembly

In another aspect, the invention provides a method for reference-assisted genome assembly. Reads from DNA proximity ligation reads on a test sample may be aligned to a reference sequence derived from a control sample to generate a combined 3D contact map. The chromosomal breakpoints and/or fusions are identified between the test sample and the reference sample to create a proxy genome assembly. Variant calling may then be used to identify one or more small-scale changes, such as indels and singe nucleotide polymorphisms, between the realigned test sample and the control reference sequence. Local reassembly is then performed on the identified variants to address the one or more small-scale changes to generate a final output genome assembly. The test sample and the reference sample may be from the same or different species, or from closely related or distantly related species. The breakpoints and fusions may be identified using one of the embodiments disclosed above. In certain example embodiments, the breakage and fusion points are examined to determine regions of synteny between the test and reference samples and/or polymorphisms. The test sample may be aligned to the same or different reference sample, or multiple test samples may be aligned to many different reference sample sequences. The breakage and fusion points may be examined to infer phylogenetic relationships between samples. In certain example embodiment, multiple reference-assisted assemblies may be prepared at the same time.

As used herein the term “control” refers to a reference standard. A control can be a known value or range of values indicative of basal levels or amounts or present in a tissue or a cell or populations thereof. A control can also be a cellular or tissue control, for example a tissue from a non-diseased state and/or exposed to different environmental conditions. A difference between a test sample and a control can be an increase or conversely a decrease. The difference can be a qualitative difference or a quantitative difference, for example a statistically significant difference.

In another aspect, the invention provides a method for genome assembly, wherein proper orientation of contigs and/or scaffolds is determined, at least in part, by the relative orientation of certain DNA motifs. The motif may be a CTCF mediated loop. The proper orientation may be determined, at least in part, from DNA proximity ligation assays, which may be used to generate a 3D contact map defining one or more contact domains, loops, compartment domains, links, compartment loops, superloops, one or more compartment interactions. The 3D contact map may also define centromere and telomere regions. In certain example embodiment, the DNA proximity ligation assay is Hi-C. In certain example embodiments, wherein massively multiplex single cell Hi-C is used to identify different subpopulations with differences in scaling and long range behavior. The DNA proximity ligation assay may be performed on synchronized populations of cells. In certain example embodiments, the cells may be synchronized in metaphase. The method may be performed on one or more cell treated to modify genome folding. Modifications may include gene editing, degradation of proteins that play a role in genome folding (such as HDAC inhibitors, Degron that target CTCF, Cohesin etc.), and/or modification of transcriptional machinery. The methods may be used to assemble transcriptomes. In certain example embodiments bisulfate treatment is applied to ligation junctions derived from a proximity ligation experiment and used to analyze proximity between DNA loci in sample, including the frequency of methylation for one or more basis in a sample.

In another aspect, the invention provides a method for genome assembly wherein the proper orientation of contigs and/or scaffolds is determined, at least in part, by the relative orientation of certain DNA motifs. In certain example embodiments, the motif is a CTCF motif. In certain example embodiments, the proper orientation of the motifs is determined, at least in part, by data from a DNA proximity ligation assay.

In another aspect, the invention provides a method for estimating the linear genomic distance between sequences in a gene comprising sequencing reads derived from DNA proximity ligation assay. The distance may be determined, at least in part, based on the frequency a given sequence forms contacts with another sequence in the set. The distance may also be determined based on the relative orientation with which a given sequence forms contacts with other sequences in the set. In certain example embodiments, the contact features are determined from DNA proximity ligation assays. In certain example embodiments, a contact map generated from the DNA proximity ligation assays may be used to derive an expected model for the linear genomic distance between sequences in a genome.

In another example embodiment, the invention provides a method for quality control analysis of genome assemblies by visually examining a contact map derived from a DNA proximity ligation assay. In certain example embodiments, the visual examination may be facilitated by a computer implemented graphical user interface, wherein the graphical user interface facilitates annotation of the genome assembly. In certain example embodiments, the contig map may span a single contig or scaffold.

The methods described herein can be used to generate a personalized genome as further exemplified in Example 8.

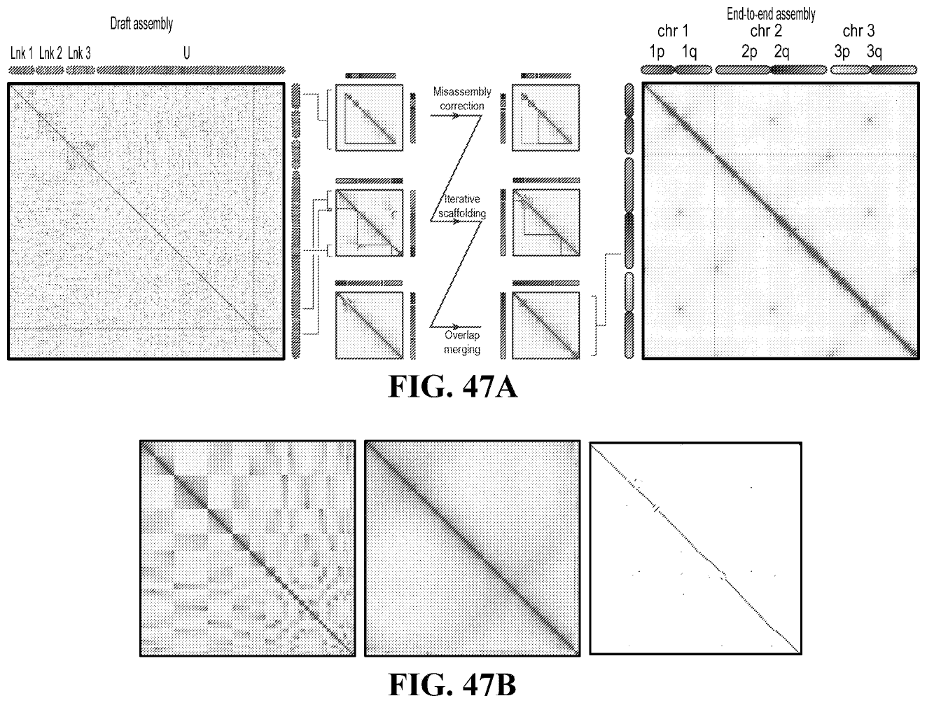

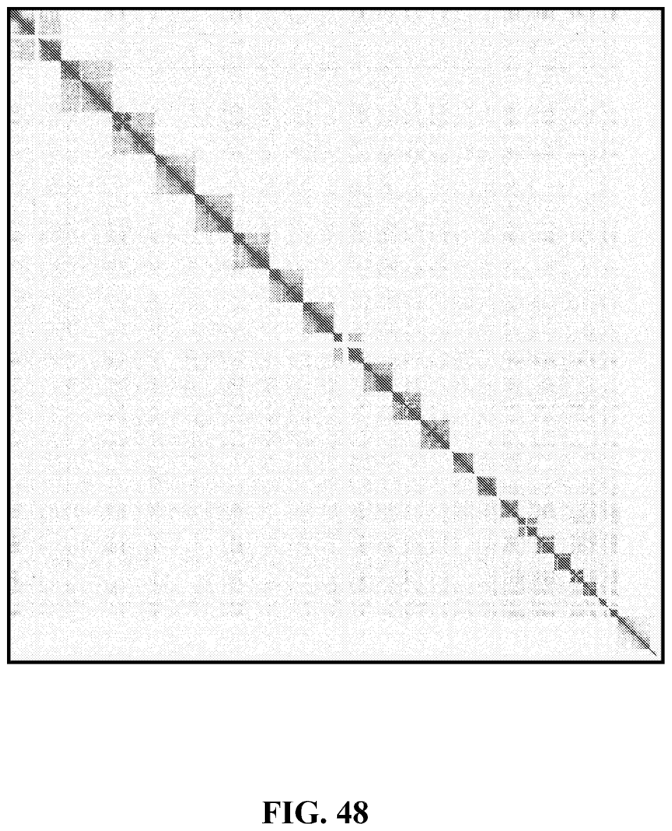

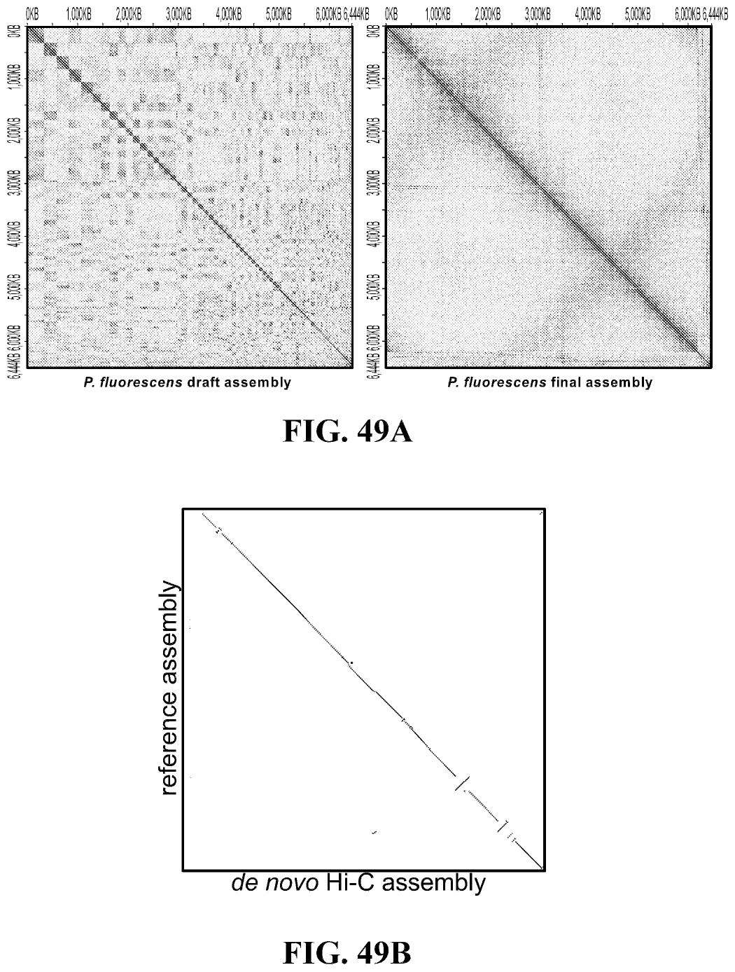

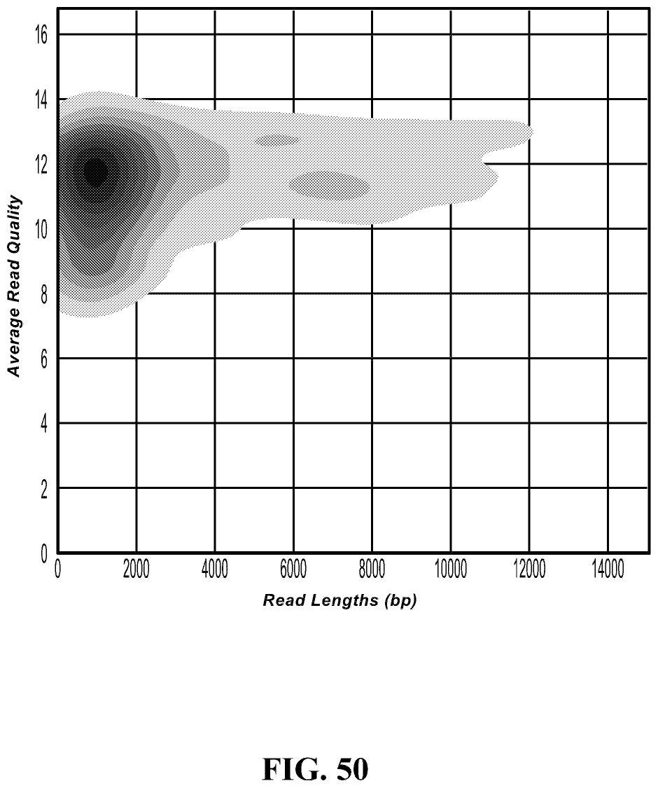

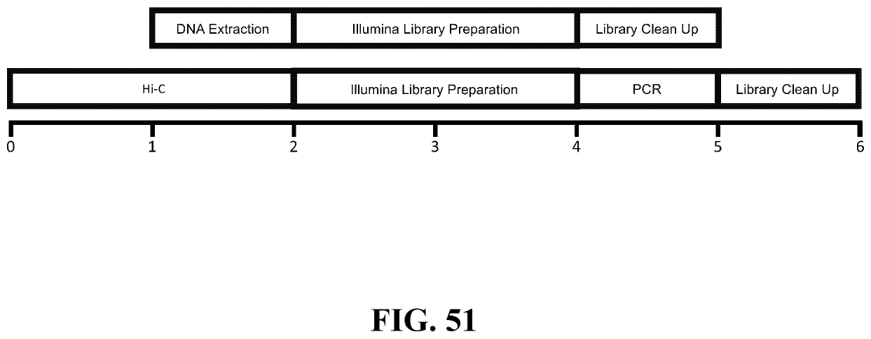

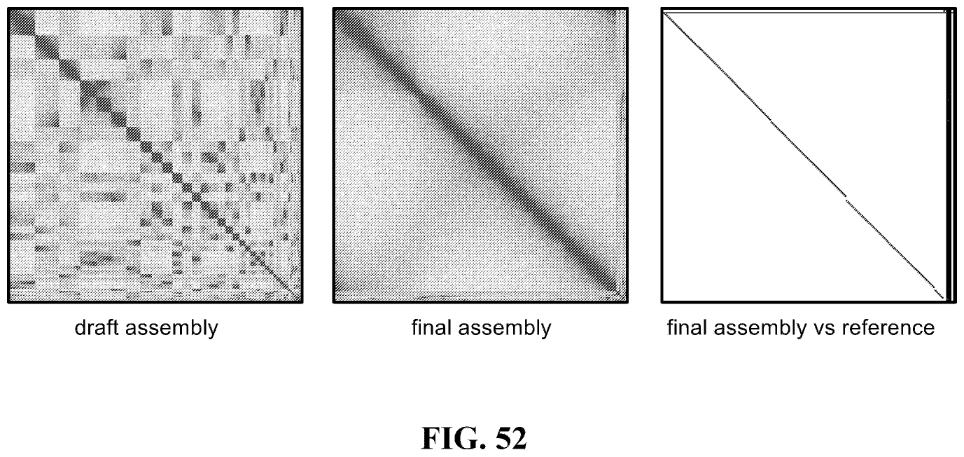

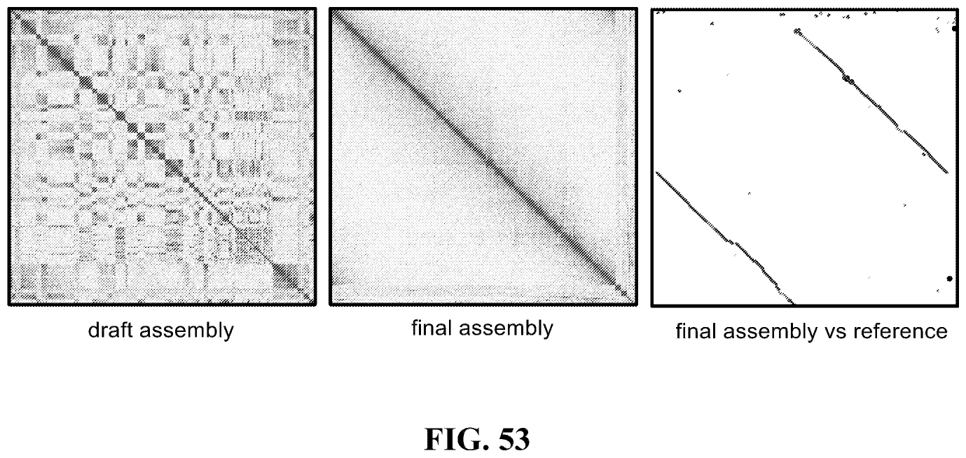

The methods disclosed herein may also be used to assemble/identify genomes in a metagenomic context. The applications include, but are not limited to, sequencing prokaryotic, eukaryotic and mixed communities from the same samples. For example, the methods may be used, among other metagenomic applications, to sequence the metagenome with the host genome, disease vectors and pathogens, and disease vectors and host etc. See FIGS. 47-53.

### Other Applications

Various embodiments of methods described herein can be used to generate data that can be analyzed using various deep learning techniques and methods for genome wide analyses as is further exemplified in Example 9.

Considering the wealth of information that can be gained using the methods described herein, with respect to genome architecture at the primary, secondary, tertiary and beyond (see Examples below), the methods disclosed herein can be used to apply genome engineering techniques for the treatment of disease as well as the study of biological questions. In some embodiments, the organizational structure of a genome is determined using the methods disclosed herein. For example, the methods disclosed herein have been demonstrated (see Example 1 and Example 12) to generate very dense contact maps. In some examples, sequences obtained using the methods disclosed herein are mapped to a genome of an organism, such as an animal, plant, fungi, or microorganism, for example a bacterial, yeast, virus and the like. In some examples, using single nucleotide polymorphisms (SNPs), diploid maps corresponding to each chromosomal homolog are constructed. These maps, as well as others that can be generated using the disclosed technology provide a picture, such as a three-dimensional picture, of genomic architecture with high resolution, such as a resolution of 1 kilobase or even lower, for example less then 50 bases, in particular 1 to 10 bp resolution.

As disclosed herein, the inventors have shown that a genome is partitioned into domains that are associated with particular patterns of histone marks that segregates into sub-compartments, distinguished by unique long-range contact patterns. Using the maps, the inventors have identified ˜10,000 distinct loops across the genome and studied their properties, including their strong association with gene activation.

### Target Ligation Junctions and Probes

Also disclosed are nucleic acids made of two or more end joined nucleic acids, target junctions, produced using the disclosed methods and amplification products thereof, such as RNA, DNA or a combination thereof. An isolated target junction is an end joined nucleic acid, wherein the junction encodes the information about the proximity of the two nucleic acid sequences that make up the target junction in a cell, for example as formed by the methods disclosed herein. The presence of an isolated target junction can be correlated with a disease state or environmental condition. For example, certain disease states may be caused and/or characterized by the differential formation of certain target junctions. Similarly, isolated target junction can be correlated to an environmental stress or state, such as but not limited to heat shock, osmolarity, hypoxia, cold, oxidative stress, radiation, starvation, a chemical (for example a therapeutic agent or potential therapeutic agent) and the like.

This disclosure also relates, to isolated nucleic acid probes that specifically bind to target junction, such as a target junction indicative of a disease state or environmental condition. To recognize a target join, a probe specifically hybridizes to the target junction both 5′ and 3′ of the site of the junction and spans the site of the target junction, or specifically hybridizes to specific target sequence with the end joined nucleic acid fragments. In some example embodiments, the specific target sequence is at least 50, at least 60, at least 70, at least 80, at least 90, at least 100, at least 110, at least 120, at least 130, at least 140, at least 150, at least 160, at least 170, at least 180, at least 190, or at least 200 base pairs long. In certain example embodiments, the specific nucleic acid sequence is within at least 50, at least 60, at least 70, at least, 80, at least 90, or at least 100 base pairs, in either the 5′ or 3′ direction, of a restriction site. In certain example embodiments, the specific nucleic sequence comprises less than ten repetitive bases. In certain other example embodiments, the GC content of the specific nucleic acid sequence is between 25% and 80%, between 40% and 70%, or between 50% and 60%.

In some embodiments, the probe is labeled, such as radiolabeled, fluorescently-labeled, biotin-labeled, enzymatically-labeled, or chemically-labeled. Non-limiting examples of the probe is an RNA probe, a DNA probe, a locked nucleic acid (LNA) probe, a peptide nucleic acid (PNA) probe, or a hybrid RNA-DNA probe. Also disclosed are sets of probes for binding to target ligation junction, as well as devices, such as nucleic acid arrays for detecting a target junction.

In embodiments, the total length of the probe, including end linked PCR or other tags, is between about 10 nucleotides and 200 nucleotides, although longer probes are contemplated. In some embodiments, the total length of the probe, including end linked PCR or other tags, is at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190 191, 192, 193, 194, 195, 196, 197, 198, 199 or 200.

In some embodiments the total length of the probe, including end linked PCR or other tags, is less than about 2000 nucleotides in length, such as less than about 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 500, 750, 1000, 1250, 1500, 1750, 2000 nucleotides in length or even greater. In some embodiments, the total length of the probe, including end linked PCR or other tags, is between about 30 nucleotides and about 250 nucleotides, for example about 90 to about 180, about 120 to about 200, about 150 to about 220 or about 120 to about 180 nucleotides in length. In some embodiments, a set of probes is used to target a specific target junction or a set of target junctions.

In some embodiments, the probe is detectably labeled, either with an isotopic or non-isotopic label, alternatively the target junction or amplification product thereof is labeled. Non-isotopic labels can, for instance, comprise a fluorescent or luminescent molecule, biotin, an enzyme or enzyme substrate or a chemical. Such labels are preferentially chosen such that the hybridization of the probe with target junction can be detected. In some examples, the probe is labeled with a fluorophore. Examples of suitable fluorophore labels are given above. In some examples, the fluorophore is a donor fluorophore. In other examples, the fluorophore is an accepter fluorophore, such as a fluorescence quencher. In some examples, the probe includes both a donor fluorophore and an accepter fluorophore. Appropriate donor/acceptor fluorophore pairs can be selected using routine methods. In one example, the donor emission wavelength is one that can significantly excite the acceptor, thereby generating a detectable emission from the acceptor.

An array containing a plurality of heterogeneous probes for the detection of target junctions are disclosed. Such arrays may be used to rapidly detect and/or identify the target junctions present in a sample, for example as part of a diagnosis. Arrays are arrangements of addressable locations on a substrate, with each address containing a nucleic acid, such as a probe. In some embodiments, each address corresponds to a single type or class of nucleic acid, such as a single probe, though a particular nucleic acid may be redundantly contained at multiple addresses. A “microarray” is a miniaturized array requiring microscopic examination for detection of hybridization. Larger “macroarrays” allow each address to be recognizable by the naked human eye and, in some embodiments, a hybridization signal is detectable without additional magnification. The addresses may be labeled, keyed to a separate guide, or otherwise identified by location.

Any sample potentially containing, or even suspected of containing, target joins may be used. A hybridization signal from an individual address on the array indicates that the probe hybridizes to a nucleotide within the sample. This system permits the simultaneous analysis of a sample by plural probes and yields information identifying the target junctions contained within the sample. In alternative embodiments, the array contains target junctions and the array is contacted with a sample containing a probe. In any such embodiment, either the probe or the target junction may be labeled to facilitate detection of hybridization.

Within an array, each arrayed nucleic acid is addressable, such that its location may be reliably and consistently determined within the at least the two dimensions of the array surface. Thus, ordered arrays allow assignment of the location of each nucleic acid at the time it is placed within the array. Usually, an array map or key is provided to correlate each address with the appropriate nucleic acid. Ordered arrays are often arranged in a symmetrical grid pattern, but nucleic acids could be arranged in other patterns (for example, in radially distributed lines, a “spokes and wheel” pattern, or ordered clusters). Addressable arrays can be computer readable; a computer can be programmed to correlate a particular address on the array with information about the sample at that position, such as hybridization or binding data, including signal intensity. In some exemplary computer readable formats, the individual samples or molecules in the array are arranged regularly (for example, in a Cartesian grid pattern), which can be correlated to address information by a computer.

An address within the array may be of any suitable shape and size. In some embodiments, the nucleic acids are suspended in a liquid medium and contained within square or rectangular wells on the array substrate. However, the nucleic acids may be contained in regions that are essentially triangular, oval, circular, or irregular. The overall shape of the array itself also may vary, though in some embodiments it is substantially flat and rectangular or square in shape.

Examples of substrates for the phage arrays disclosed herein include glass (e.g., functionalized glass), Si, Ge, GaAs, GaP, SiO₂, SiN₄, modified silicon nitrocellulose, polyvinylidene fluoride, polystyrene, polytetrafluoroethylene, polycarbonate, nylon, fiber, or combinations thereof. Array substrates can be stiff and relatively inflexible (for example glass or a supported membrane) or flexible (such as a polymer membrane). One commercially available product line suitable for probe arrays described herein is the Microlite line of MICROTITER® plates available from Dynex Technologies UK (Middlesex, United Kingdom), such as the Microlite 1+96-well plate, or the 384 Microlite+384-well plate.

Addresses on the array should be discrete, in that hybridization signals from individual addresses can be distinguished from signals of neighboring addresses, either by the naked eye (macroarrays) or by scanning or reading by a piece of equipment or with the assistance of a microscope (microarrays).

### Systems

Also disclosed is a system wherein information from one or more ligation junctions is used to identify regions of the genome that control or modulate spatial proximity relationships between nucleic acids. In some embodiments, the genomic regions identified establish chromatin loops. In some embodiments, the genomic regions identified demarcate or establish contiguous intervals of chromatin that display elevated proximity between loci within the intervals.

Further disclosed is a system for visualizing, such as system comprising hardware and/or software, the information from one or more ligation junctions. In some examples, the information from one or more ligation junctions is represented in a matrix with entries indicating frequency of interaction. In some examples, a user can dynamically zoom in and out, viewing interactions between smaller or larger pieces of the genome. In some examples, interaction matrices and other 1-D data vectors can be viewed and compared simultaneously. In some examples, the annotations of features can be superimposed on interaction matrices. In some examples, multiple interaction matrices can be simultaneously viewer and compared.

This disclosure also provides integrated systems for high-throughput testing, or automated testing. The systems typically include a robotic armature that transfers fluid from a source to a destination, a controller that controls the robotic armature, a detector, a data storage unit that records detection, and an assay component such as a microtiter dish comprising a well having a reaction mixture for example media.

As used herein the term “high throughput technique” refers to a combination of methods, robotics, data processing and control software, liquid handling devices, and detectors that allows the rapid screening of potential reagents, conditions, or targets in a short period of time, for example in less than 24, less than 12, less than 6 hours, or even less than 1 hour.

### Kits

The nucleic acid probes, such as probes for specifically binding to a target junction, and other reagents disclosed herein for use in the disclosed methods can be supplied in the form of a kit. In such a kit, an appropriate amount of one or more of the nucleic acid probes is provided in one or more containers or held on a substrate. A nucleic acid probe may be provided suspended in an aqueous solution or as a freeze-dried or lyophilized powder, for instance. The container(s) in which the nucleic acid(s) are supplied can be any conventional container that is capable of holding the supplied form, for instance, microfuge tubes, ampoules, or bottles. The kits can include either labeled or unlabeled nucleic acid probes for use in detection, of a target junction. The amount of nucleic acid probe supplied in the kit can be any appropriate amount, and may depend on the target market to which the product is directed. A kit may contain more than one different probe, such as 2, 3, 4, 5, 6, 7, 8, 9, 10, 11, 12, 13, 14, 15, 20, 25, 50, 100, or more probes. The instructions may include directions for obtaining a sample, processing the sample, preparing the probes, and/or contacting each probe with an aliquot of the sample. In certain embodiments, the kit includes an apparatus for separating the different probes, such as individual containers (for example, microtubules) or an array substrate (such as, a 96-well or 384-well microtiter plate). In particular embodiments, the kit includes prepackaged probes, such as probes suspended in suitable medium in individual containers (for example, individually sealed EPPENDORF® tubes) or the wells of an array substrate (for example, a 96-well microtiter plate sealed with a protective plastic film). In some embodiments, kits also may include the reagents necessary to carry out methods disclosed herein. In other particular embodiments, the kit includes equipment, reagents, and instructions for the methods disclosed herein.

### Genome Engineering

In certain embodiments, a specific sequence identified at a chromatin loop anchor according to the present invention can be targeted using a genome modifying agent (e.g., CTCF dependent or CTCF independent loops). In certain embodiments, a cell is modified to treat a disease, to model a disease, or to study a biological process. For example, a transcription factor binding site or a specific regulatory sequence (e.g., a sequence in contact with a promoter, a sequence within an enhancer, or an activator binding site). In certain embodiments, a specific variant associated with a disease is modified to treat the disease. In certain embodiments, a gene associated according to the methods described herein with a disease causing variant is modified. For example, a variant present in an enhancer or regulatory sequence that is in contact with a gene. In certain embodiments, a cell is modified in vivo, ex vivo or in vitro.

A method of the invention may be used to create a plant, an animal or cell that may be used to model and/or study genetic or epigenetic conditions of interest, such as a through a model of mutations of interest or a as a disease model. As used herein, “disease” refers to a disease, disorder, or indication in a subject. For example, a method of the invention may be used to create an animal or cell that comprises a modification in one or more nucleic acid sequences associated with a disease, or a plant, animal or cell in which the expression of one or more nucleic acid sequences associated with a disease are altered. Such a nucleic acid sequence may encode a disease associated protein sequence or may be a disease associated control sequence. Accordingly, it is understood that in embodiments of the invention, a plant, subject, patient, organism or cell can be a non-human subject, patient, organism or cell. Thus, the invention provides a plant, animal or cell, produced by the present methods, or a progeny thereof. The progeny may be a clone of the produced plant or animal, or may result from sexual reproduction by crossing with other individuals of the same species to introgress further desirable traits into their offspring. The cell may be in vivo or ex vivo in the cases of multicellular organisms, particularly animals or plants. In the instance where the cell is in cultured, a cell line may be established if appropriate culturing conditions are met and preferably if the cell is suitably adapted for this purpose (for instance a stem cell). Bacterial cell lines produced by the invention are also envisaged. Hence, cell lines are also envisaged.

### Genetic Modifying Agents

In certain embodiments, the genetic modifying agent may comprise a CRISPR system, a zinc finger nuclease system, a TALEN, a meganuclease or RNAi system.

### CRISPR-Cas Modification

In some embodiments, a polynucleotide of the present invention described elsewhere herein can be modified using a CRISPR-Cas and/or Cas-based system (e.g., genomic DNA or mRNA, preferably, for a disease gene). The nucleotide sequence may be or encode one or more components of a CRISPR-Cas system. For example, the nucleotide sequences may be or encode guide RNAs. The nucleotide sequences may also encode CRISPR proteins, variants thereof, or fragments thereof.

In general, a CRISPR-Cas or CRISPR system as used herein and in other documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g., CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

CRISPR-Cas systems can generally fall into two classes based on their architectures of their effector molecules, which are each further subdivided by type and subtype. The two classes are Class 1 and Class 2. Class 1 CRISPR-Cas systems have effector modules composed of multiple Cas proteins, some of which form crRNA-binding complexes, while Class 2 CRISPR-Cas systems include a single, multi-domain crRNA-binding protein.

In some embodiments, the CRISPR-Cas system that can be used to modify a polynucleotide of the present invention described herein can be a Class 1 CRISPR-Cas system. In some embodiments, the CRISPR-Cas system that can be used to modify a polynucleotide of the present invention described herein can be a Class 2 CRISPR-Cas system.

### Class 1 CRISPR-Cas Systems

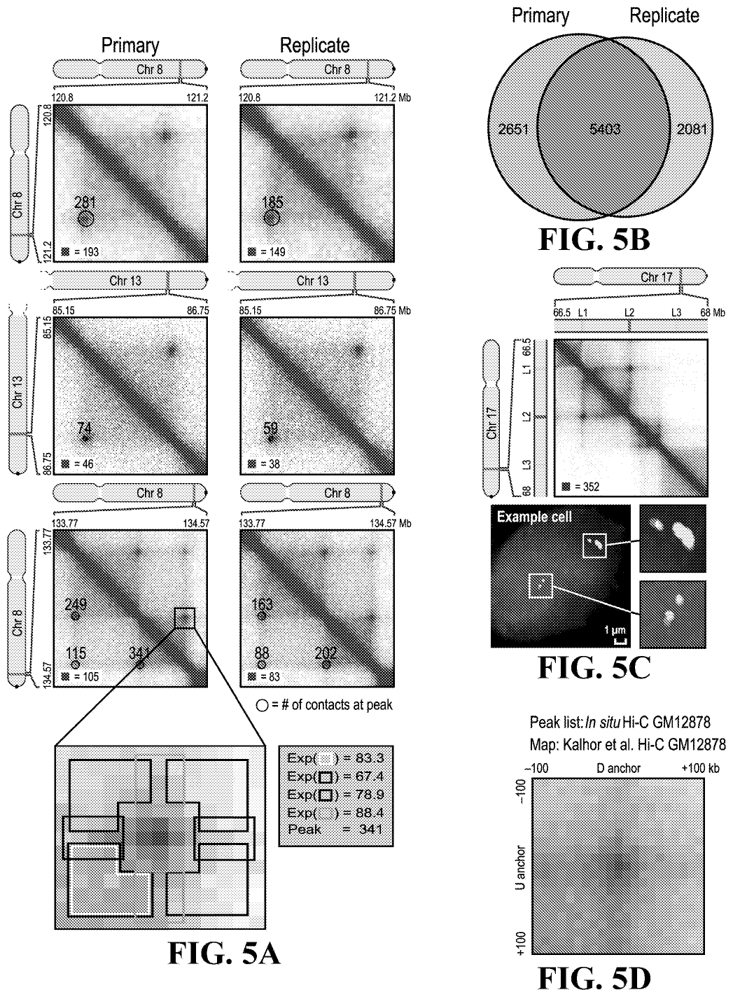

In some embodiments, the CRISPR-Cas system that can be used to modify a polynucleotide of the present invention described herein can be a Class 1 CRISPR-Cas system. Class 1 CRISPR-Cas systems are divided into Types I, II, and IV. Makarova et al. 2020. Nat. Rev. 18: 67-83., particularly as described in FIG. 1. Type I CRISPR-Cas systems are divided into 9 subtypes (I-A, I-B, I-C, I-D, I-E, I-F1, I-F2, I-F3, and IG). Makarova et al., 2020. Class 1, Type I CRISPR-Cas systems can contain a Cas3 protein that can have helicase activity. Type III CRISPR-Cas systems are divided into 6 subtypes (III-A, III-B, III-E, and III-F). Type III CRISPR-Cas systems can contain a Cas10 that can include an RNA recognition motif called Palm and a cyclase domain that can cleave polynucleotides. Makarova et al., 2020. Type IV CRISPR-Cas systems are divided into 3 subtypes. (IV-A, IV-B, and IV-C). Makarova et al., 2020. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al. 2018. The CRISPR Journal, v. 1, n5, FIG. 5.

The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g., Cas 4, DNA nuclease), CRISPR associated Rossman fold (CARF) domain containing proteins, and/or RNA transcriptase.

The backbone of the Class 1 CRISPR-Cas system effector complexes can be formed by RNA recognition motif domain-containing protein(s) of the repeat-associated mysterious proteins (RAMPs) family subunits (e.g., Cas 5, Cas6, and/or Cas7). RAMP proteins are characterized by having one or more RNA recognition motif domains. In some embodiments, multiple copies of RAMPs can be present. In some embodiments, the Class I CRISPR-Cas system can include 1, 2, 3, 4, 5, 6, 7, 8, 9, 10, 11, 12 or more Cas5, Cas6, and/or Cas 7 proteins. In some embodiments, the Cas6 protein is an RNAse, which can be responsible for pre-crRNA processing. When present in a Class 1 CRISPR-Cas system, Cas6 can be optionally physically associated with the effector complex.

Class 1 CRISPR-Cas system effector complexes can, in some embodiments, also include a large subunit. The large subunit can be composed of or include a Cas8 and/or Cas10 protein. See, e.g., FIGS. 1 and 2. Koonin E V, Makarova K S. 2019. Phil. Trans. R. Soc. B 374: 20180087, DOI: 10.1098/rstb.2018.0087 and Makarova et al. 2020.

Class 1 CRISPR-Cas system effector complexes can, in some embodiments, include a small subunit (for example, Cas11). See, e.g., FIGS. 1 and 2. Koonin E V, Makarova K S. 2019 Origins and Evolution of CRISPR-Cas systems. Phil. Trans. R. Soc. B 374: 20180087, DOI: 10.1098/rstb.2018.0087.

In some embodiments, the Class 1 CRISPR-Cas system can be a Type I CRISPR-Cas system. In some embodiments, the Type I CRISPR-Cas system can be a subtype I-A CRISPR-Cas system. In some embodiments, the Type I CRISPR-Cas system can be a subtype I-B CRISPR-Cas system. In some embodiments, the Type I CRISPR-Cas system can be a subtype I-C CRISPR-Cas system. In some embodiments, the Type I CRISPR-Cas system can be a subtype I-D CRISPR-Cas system. In some embodiments, the Type I CRISPR-Cas system can be a subtype I-E CRISPR-Cas system. In some embodiments, the Type I CRISPR-Cas system can be a subtype I-F1 CRISPR-Cas system. In some embodiments, the Type I CRISPR-Cas system can be a subtype I-F2 CRISPR-Cas system. In some embodiments, the Type I CRISPR-Cas system can be a subtype I-F3 CRISPR-Cas system. In some embodiments, the Type I CRISPR-Cas system can be a subtype I-G CRISPR-Cas system. In some embodiments, the Type I CRISPR-Cas system can be a CRISPR Cas variant, such as a Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems as previously described.

In some embodiments, the Class 1 CRISPR-Cas system can be a Type III CRISPR-Cas system. In some embodiments, the Type III CRISPR-Cas system can be a subtype III-A CRISPR-Cas system. In some embodiments, the Type III CRISPR-Cas system can be a subtype III-B CRISPR-Cas system. In some embodiments, the Type III CRISPR-Cas system can be a subtype III-C CRISPR-Cas system. In some embodiments, the Type III CRISPR-Cas system can be a subtype III-D CRISPR-Cas system. In some embodiments, the Type III CRISPR-Cas system can be a subtype III-E CRISPR-Cas system. In some embodiments, the Type III CRISPR-Cas system can be a subtype III-F CRISPR-Cas system.

In some embodiments, the Class 1 CRISPR-Cas system can be a Type IV CRISPR-Cas-system. In some embodiments, the Type IV CRISPR-Cas system can be a subtype IV-A CRISPR-Cas system. In some embodiments, the Type IV CRISPR-Cas system can be a subtype IV-B CRISPR-Cas system. In some embodiments, the Type IV CRISPR-Cas system can be a subtype IV-C CRISPR-Cas system.

The effector complex of a Class 1 CRISPR-Cas system can, in some embodiments, include a Cas3 protein that is optionally fused to a Cas2 protein, a Cas4, a Cas5, a Cas6, a Cas7, a Cas8, a Cas10, a Cash 1, or a combination thereof. In some embodiments, the effector complex of a Class 1 CRISPR-Cas system can have multiple copies, such as 1, 2, 3, 4, 5, 6, 7, 8, 9, 10, 11, 12, 13, or 14, of any one or more Cas proteins.

### Class 2 CRISPR-Cas Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), CasX, and/or Cas14.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SETT/9), a translation initiation domain, a transcriptional repression domain (e.g., a KRAB domain, NuE domain, NcoR domain, and a SID domain such as a SID4× domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (WO 2019/005884, WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Patent Publication No. WO 2019/018423.

### Split CRISPR-Cas Systems

In some embodiments, the CRISPR-Cas system is a split CRISPR-Cas system. See e.g., Zetche et al., 2015. Nat. Biotechnol. 33(2): 139-142 and WO 2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein is attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C•G base pair into a T•A base pair (Komor et al. 2016. Nature. 533:420-424; Nishida et al. 2016. Science. 353; and Li et al. Nat. Biotech. 36:324-327) and ABEs convert an A•T base pair to a G•C base pair. Collectively, CBEs and ABEs can mediate all four possible transition mutations (C to T, A to G, T to C, and G to A). Rees and Liu. 2018. Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471. Base editors may be further engineered to optimize conversion of nucleotides (e.g. A:T to G:C). Richter et al. 2020. Nature Biotechnology. doi.org/10.1038/s41587-020-0453-z.

Other Example Type V base editing systems are described in WO 2018/213708, WO 2018/213726, PCT/US2018/067207, PCT/US2018/067225, and PCT/US2018/067307 which are incorporated by referenced herein.

In certain example embodiments, the base editing system may be a RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA based editor may be used to delete or introduce a post-translation modification site in the expressed mRNA. In contrast to DNA base editors, whose edits are permanent in the modified cell, RNA base editors can provide edits where finer temporal control may be needed, for example in modulating a particular immune response. Example Type VI RNA-base editing systems are described in Cox et al. 2017. Science 358: 1019-1027, WO 2019/005884, WO 2019/005886, WO 2019/071048, PCT/US20018/05179, PCT/US2018/067207, which are incorporated herein by reference. An example FnCas9 system that may be adapted for RNA base editing purposes is described in WO 2016/106236, which is incorporated herein by reference.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

### Prime Editors

In some embodiments, a polynucleotide of the present invention described elsewhere herein can be modified using a prime editing system (See e.g., Anzalone et al. 2019. Nature. 576: 149-157). Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion, and combinations thereof. Generally, a prime editing system, as exemplified by PE1, PE2, and PE3 (Id.), can include a reverse transcriptase fused or otherwise coupled or associated with an RNA-programmable nickase, and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRIPSR-Cas systems along with few byproducts and greater or similar efficiency as compared to traditional CRISPR-Cas systems.

In some embodiments, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g., a prime editing guide molecule or peg guide molecule) directly into the target site in the target polynucleotide. See e.g., Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g., is a Cas9 nickase). In some embodiments, the Cas polypeptide is fused to the reverse transcriptase. In some embodiments, the Cas polypeptide is linked to the reverse transcriptase.

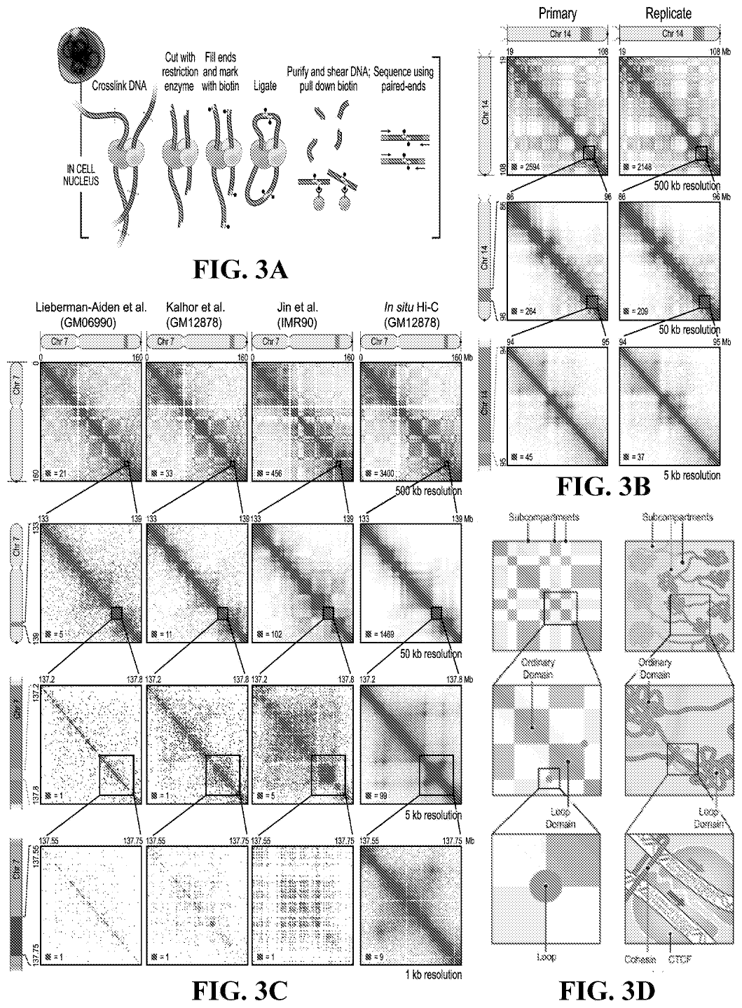

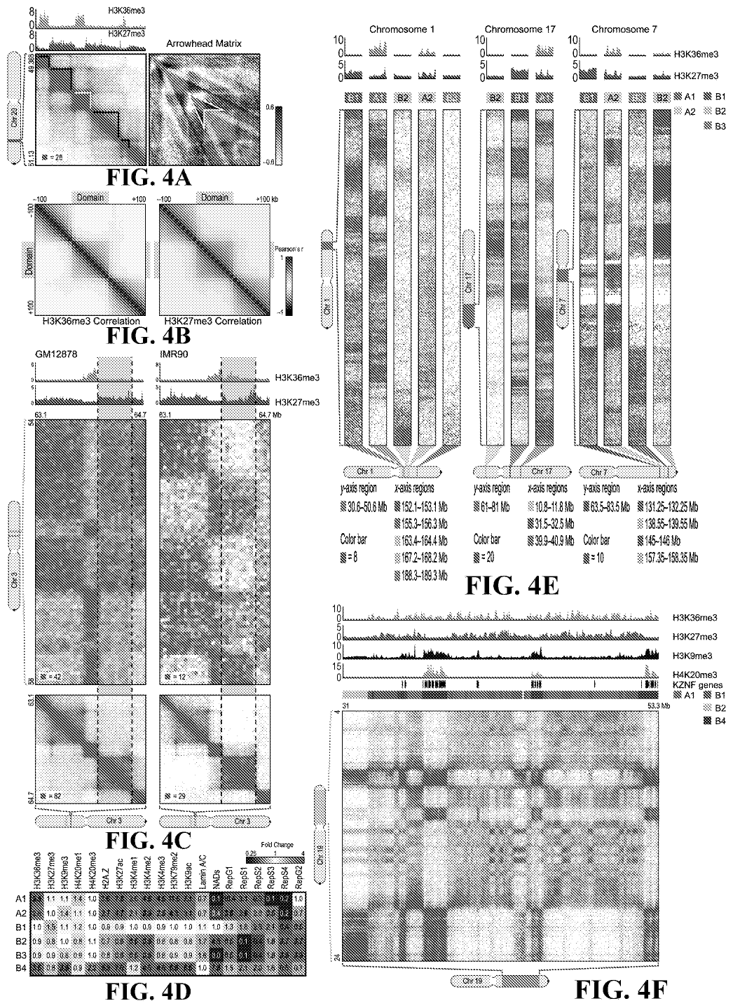

In some embodiments, the prime editing system can be a PE1 system or variant thereof, a PE2 system or variant thereof, or a PE3 (e.g., PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4,

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CRISPR Associated Transposase (CAST) Systems

In some embodiments, a polynucleotide of the present invention described elsewhere herein can be modified using a CRISPR Associated Transposase (“CAST”) system. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference.

### Guide Molecules

The CRISPR-Cas or Cas-Based system described herein can, in some embodiments, include one or more guide molecules. The terms guide molecule, guide sequence and guide polynucleotide, refer to polynucleotides capable of guiding Cas to a target genomic locus and are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarily, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), Clustal W, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in PCT US2019/045582, specifically paragraphs [0178]-[0333], which is incorporated herein by reference.

### Target Sequences, PAMs, and PFSs

### Target Sequences

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table A below shows several Cas polypeptides and the PAM sequence they recognize.

TABLE A
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Casl1a (Cpf1) (including | TTTV |
| LbCpf1 and AsCpf1) | |
| Cas12b (C2c1) | TTT, TTA, and TTC |
| Cas12c (C2c3) | TA |
| Cas12d (CasY) | TA |
| Cas12e (Casx) | 5′-TTCN-3′ |

In a preferred embodiment, the CRISPR effector protein may recognize a 3′ PAM. In certain embodiments, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead, such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Zinc Finger Nucleases

In some embodiments, the polynucleotide is modified using a Zinc Finger nuclease or system thereof. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to FokI cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### TALE Nucleases

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁-(X₁₂X₁₃)-X₁₄₋₃₃ or 34 or 35, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂X₁₃)-X₁₄₋₃₃ or 34 or 35)_{z}, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 1)
M D P I R S R T P S P A R E L L S G P Q P D G V Q
P T A D R G V S P P A G G P L D G L P A R R T M S
R T R L P S P P A P S P A F S A D S F S D L L R Q
F D P S L F N T S L F D S L P P F G A H H T E A A
T G E W D E V Q S G L R A A D A P P P T M R V A V
T A A R P P R A K P A P R R R A A Q P S D A S P A
A Q V D L R T L G Y S Q Q Q Q E K I K P K V R S T
V A Q H H E A L V G H G F T H A H I V A L S Q H P
A A L G T V A V K Y Q D M I A A L P E A T H E A I
V G V G K Q W S G A R A L E A L L T V A G E L R G
P P L Q L D T G Q L L K I A K R G G V T A V E A V
H A W R N A L T G A P L N

An exemplary amino acid sequence of a C-terminal capping region is:

(SEQ ID NO: 2)
R P A L E S I V A Q L S R P D P A L A A L T N D H
L V A L A C L G G R P A L D A V K K G L P H A P A
L I K R T N R R I P E R T S H R V A D H A Q V V R
V L G F F Q C H S H P A Q A F D D A M T Q F G M S
R H G L L Q L F R R V G V T E L E A R S G T L P P
A S Q R W D R I L Q A S G M K R A K P S P T S T Q
T P D Q A S L H A F A D S L E R D L D A P S P M H
E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain an N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full-length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4× domain or a Krüppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated by reference.

### Sequences Related to Nucleus Targeting and Transportation

In some embodiments, one or more components (e.g., the Cas protein and/or deaminase, Zn Finger protein, TALE, or meganuclease) in the composition for engineering cells may comprise one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO: 3) or PKKKRKVEAS (SEQ ID NO: 4); the NLS from nucleoplasmin (e.g., the nucleoplasmin bipartite NLS with the sequence KRPAATKKAGQAKKKK (SEQ ID NO: 5)); the c-myc NLS having the amino acid sequence PAAKRVKLD (SEQ ID NO: 6) or RQRRNELKRSP (SEQ ID NO: 7); the hRNPA1 M9 NLS having the sequence NQSSNFGPMKGGNFGGRSSGPYGGGGQYFAKPRNQGGY (SEQ ID NO: 8); the sequence RMRIZFKNKGKDTAELRRRRVEVSVELRKAKKDEQILKRRNV (SEQ ID NO: 9) of the IBB domain from importin-alpha; the sequences VSRKRPRP (SEQ ID NO: 10) and PPKKARED (SEQ ID NO: 11) of the myoma T protein; the sequence PQPKKKPL (SEQ ID NO: 12) of human p53; the sequence SALI AP (SEQ ID NO: 13) of mouse c-abl IV; the sequences DRLRR (SEQ ID NO: 14) and PKQKKRK (SEQ ID NO: 15) of the influenza virus NS1; the sequence RKLKKKIKKL (SEQ ID NO: 16) of the Hepatitis virus delta antigen; the sequence REKKKFLKRR (SEQ ID NO: 17) of the mouse Mx1 protein; the sequence KRKGDEVDGVDEVAKKKSKK (SEQ ID NO: 18) of the human poly(ADP-ribose) polymerase; and the sequence RKCLQAGMNLEARKTKK (SEQ ID NO: 19) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein, or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

In certain embodiments, the CRISPR-Cas protein and the deaminase protein are delivered to the cell or expressed within the cell as separate proteins. In these embodiments, each of the CRISPR-Cas and deaminase protein can be provided with one or more NLSs as described herein. In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In certain embodiments, guides of the disclosure comprise specific binding sites (e.g., aptamers) for adapter proteins, which may be linked to or fused to an nucleotide deaminase or catalytic domain thereof. When such a guide forms a CRISPR complex (e.g., CRISPR-Cas protein binding to guide and target) the adapter proteins bind and, the nucleotide deaminase or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g., due to steric hindrance within the three dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the nucleotide deaminase protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively or additionally, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

### Templates

In some embodiments, the composition for engineering cells comprise a template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas protein mediated cleavage event. In an embodiment, the template nucleic acid may include sequence that corresponds to both, a first site on the target sequence that is cleaved in a first Cas protein mediated event, and a second site on the target sequence that is cleaved in a second Cas protein mediated event.

In certain embodiments, the template nucleic acid can include sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include sequence which, when integrated, results in: decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include sequence which results in: a change in sequence of 1, 2, 3, 4, 5, 6, 7, 8, 9, 10, 11, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 110+/−10, 120+/−10, 130+/−10, 140+/−10, 150+/−10, 160+/−10, 170+/−10, 180+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, 150+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g., about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may be designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

In certain embodiments, a template nucleic acid for correcting a mutation may be designed for use with a homology-independent targeted integration system. Suzuki et al. describe in vivo genome editing via CRISPR/Cas9 mediated homology-independent targeted integration (2016, Nature 540:144-149). Schmid-Burgk, et al. describe use of the CRISPR-Cas9 system to introduce a double-strand break (DSB) at a user-defined genomic location and insertion of a universal donor DNA (Nat Commun. 2016 Jul. 28; 7:12338). Gao, et al. describe “Plug-and-Play Protein Modification Using Homology-Independent Universal Genome Engineering” (Neuron. 2019 Aug. 21; 103(4):583-597).

### RNAi

In some embodiments, the genetic modulating agents may be interfering RNAs. In certain embodiments, diseases caused by a dominant mutation in a gene is targeted by silencing the mutated gene using RNAi. In some cases, the nucleotide sequence may comprise coding sequence for one or more interfering RNAs. In certain examples, the nucleotide sequence may be interfering RNA (RNAi). As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e., although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

In certain embodiments, a modulating agent may comprise silencing one or more endogenous genes. As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA”, used interchangeably herein, are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

Further embodiments are illustrated in the following Examples which are given for illustrative purposes only and are not intended to limit the scope of the invention.

### EXAMPLES

### Example 1—In Situ Hi-C

The inventors used the disclosed methods, termed in situ Hi-C (an improved method for probing the three-dimensional architecture of Genomes) to construct haploid and diploid maps of nine cell types. The densest, in human lymphoblastoid cells, contains 4.9 billion contacts, achieving 1-kilobase resolution. The inventors found that genomes are partitioned into local domains, which are associated with distinct patterns of histone marks and segregate into six sub-compartments. The inventors identified ˜10,000 loops. These loops frequently link promoters and enhancers, correlate with gene activation, and show conservation across cell types and species. Loop anchors typically occur at domain boundaries and bind CTCF. CTCF sites at loop anchors occur predominantly (>90%) in a convergent orientation, with the asymmetric motifs ‘facing’ one another. The inactive X-chromosome splits into two massive domains and contains large loops anchored at CTCF-binding repeats.

The spatial organization of the human genome is known to play an important role in the transcriptional control of genes (Bickmore, Annual review of genomics and human genetics 14, 67-84, 2013; Cremer and Cremer, Nature Rev Genet 2, 292-301, 2001; Sexton et al., Nature structural & molecular biology 14, 1049-1055, 2007). Yet important questions remain, like how distal regulatory elements, such as enhancers, affect promoters and how insulators can abrogate these effects (Banerji et al., Cell 27, 299-308, 1981; Blackwood and Kadonaga, Science (New York, N.Y.) 281, 60-63, 1998; Gaszner and Felsenfeld, Nature Reviews: Genetics 7, 703-713, 2006). Both phenomena are thought to involve the formation of protein-mediated “loops” that bring pairs of genomic sites that lie far apart along the linear genome into proximity (Schleif, Annual review of biochemistry 61, 199-223, 1992).

Over the past quarter-century, various methods have emerged to assess the three-dimensional architecture of the nucleus in vivo (Gerasimova et al., Molecular cell 6, 1025-1035, 2000; Mukherjee et al., Cell 52, 375-383, 1988), including nuclear ligation assay and chromosome conformation capture (3C), which analyze contacts made by a single locus (Cullen et al., Science 261, 203-206, 1993; Dekker et al., Science 295, 1306-1311, 2002; Murrell et al., Nature genetics 36, 889-893, 2004; Tolhuis et al., Molecular cell 10, 1453-1465, 2002), extensions such as 5 C. for examining several loci simultaneously (Dostie et al., Genome research 16, 1299-1309, 2006), and methods such as CHIA-PET for examining all loci bound by a specific protein (Fullwood et al., Nature 462, 58-64, 2009). The inventors had previously developed Hi-C, which combines DNA-DNA proximity ligation with high throughput sequencing to interrogate all pairs of loci across a genome (Lieberman-Aiden et al., Science 326, 289-293, 2009).

Disclosed herein is a new and unique method, dubbed in situ Hi-C, in which proximity ligation is performed in intact nuclei. The protocol facilitates generation of much denser Hi-C maps. The maps reported here comprise 5 terabases of sequence data recording over 15 billion contacts; they are larger, by an order of magnitude, than all published Hi-C datasets combined. Using single nucleotide polymorphisms (SNPs), Applicants also construct diploid maps corresponding to each chromosomal homolog. The maps provide a picture of genomic architecture with resolution down to 1 kilobase. They show that the genome is partitioned into domains that are associated with particular patterns of histone marks and that segregate into six sub-compartments, distinguished by unique long range contact patterns. Using the maps, the inventors have identified 10,000 distinct loops across the genome and study their properties, including their strong association with gene activation. Strikingly, the vast majority of loop anchors bind CTCF. Moreover, the two CTCF motifs that occur at the anchors of a loop are found in a convergent orientation—that is, with the asymmetric CTCF motifs ‘facing’ one another—over 90% of the time. The diploid maps show that the inactive Xchromosome is partitioned into two massive domains, and contains large loops anchored at CTCF-binding repeats.

### In Situ Hi-C Methodology and Maps

As implemented in this Example, the disclosed in situ Hi-C protocol involves cross-linking cells with formaldehyde; permeabilizing them with nuclei intact; digesting DNA with a suitable 4-cutter restriction enzyme (such as MboI); filling the 5′-overhangs while incorporating a biotinylated nucleotide; ligating the resulting blunt-end fragments; shearing the DNA; capturing the biotinylated ligation junctions with streptavidin beads; and analyzing the resulting fragments with paired-end sequencing (FIG. 3A).

The protocol has three major advantages over the original Hi-C protocol (here called dilution Hi-C). First, in situ ligation reduces the frequency of spurious contacts due to random ligation in dilute solution—as evidenced by a lower frequency of junctions between mitochondrial and nuclear DNA. Second, the protocol is much faster, requiring three days instead of seven. Third, it enables higher resolution and more efficient cutting of chromatinized DNA, for instance, through the use of a 4-cutter (MboI) rather than a 6-cutter (typically, HindIII).

A Hi-C map is a list of DNA-DNA contacts produced by a Hi-C experiment. By partitioning the linear genome into “loci” of fixed size (e.g., bins of 1 Mb or 1 Kb), the Hi-C map can be represented as a “contact matrix” M, where the entry Mi,j is the number of contacts observed between locus Li and locus Lj. (A “contact” is a read pair that remains after Applicants exclude reads that do not align uniquely to the genome, that correspond to unligated fragments, or that are duplicates.) The contact matrix can be visualized as a heatmap, whose entries are called “pixels”. An “interval” refers to a (one-dimensional) set of consecutive loci; the contacts between two intervals thus form a “rectangle” or “square” in the contact matrix. “Matrix resolution” is defined as the locus size used to construct a particular contact matrix and “map resolution” as the smallest locus size such that 80% of loci have at least 1000 contacts. The map resolution describes the finest scale at which one can reliably discern local features in the data.

### Contact Maps Spanning 9 Cell Lines Containing Over 15 Billion Contacts.

The inventors constructed in situ Hi-C maps of 9 cell lines in human and mouse. Whereas the original Hi-C experiments had a map resolution of 1 Mb, these maps have a resolution of 1 Kb or 5 Kb, demonstrating the surprising improvement. The largest map, in human GM12878 B-lymphoblastoid cells, aggregates the results of nine biological replicate experiments derived from independent cell cultures. It contains 4.9 billion pairwise contacts and has map resolution of 950 bp (“kilobase resolution”). This map was used to construct contact matrices with locus sizes ranging from 2.5 Mb to 1 Kb. The inventors also generated eight in situ Hi-C maps at 5 kb resolution, using cell lines representing all human germ layers (IMR90, HMEC, NHEK, K562, HUVEC, HeLa, and KBM7) as well as mouse Blymphoblasts (CH12-LX). Each of these maps contains between 395M and 1.1B contacts. To test reproducibility, a comparison was made of “primary” GM12878 map (2.6 billion contacts from a single culture) to a “replicate” map (2.3 billion contacts aggregated from experiments on eight other samples). The results were strongly correlated both visually and statistically (Pearson's R>0.998, 0.996, 0.96 and 0.85 at matrix resolutions of 500, 50, 5, and 1 Kb; P-values throughout are negligible unless stated) (FIG. 1B-D). Biological replicates were compared in IMR90, HMEC, K562, KBM7, and CH12-LX with similar results. To ensure that the results were comparable with those of previous Hi-C experiments, an original dilution Hi-C protocol was used to generate a map of GM12878 with 3.2 billion contacts; the in situ and dilution Hi-C showed high reproducibility (R>0.96,0.90,0.87 at 500,50,25 Kb). This procedure was repeated in IMR90, HMEC, NHEK, HUVEC, CH12-LX with similar results. The inventors also performed 112 supplementary Hi-C experiments using three different protocols (in situ Hi-C, dilution Hi-C, and Tethered Conformation Capture) while varying a wide array of conditions such as crosslinking time, restriction enzyme, ligation volume/time, and biotinylated nucleotide. The experiments demonstrated that the findings presented herein were robust to particular experimental conditions (see the sections on loop calling). In total, 201 independent Hi-C experiments were successfully performed. To identify fine-scale features in Hi-C maps, it is essential to account for non-uniformities in coverage due to the number of restriction sites at a locus or the accessibility of those sites to cutting (Cournac et al., BMC genomics 13, 436, 2012; Hu et al., Bioinformatics (Oxford, England) 28, 3131-3133, 2012; Imakaev et al., Nature methods 9, 999-1003, 2012; Lieberman-Aiden et al., Science 326, 289-293, 2009; Yaffe and Tanay, Nature genetics 43, 1059-1065, 2011). Either circumstance would increase the number of restriction fragments at the locus available for ligation, and thus the frequency of contacts involving the locus and any other locus. These non-uniformities were accounted for by normalizing each contact matrix using a matrix-balancing algorithm due to Knight and Ruiz (Knight and Ruiz, IMA Journal of Numerical Analysis, 2012). Three other published Hi-C bias-correction methods were also used (Cournac et al., BMC genomics 13, 436, 2012; Imakaev et al., Nature methods 9, 999-1003, 2012; Lieberman-Aiden et al., Science 326, 289-293, 2009); all produced similar results.

The Genome is Partitioned into Small Domains with Consistent Patterns of Chromatin.

It was next sought to use the vastly higher (200- to 1000-fold) map resolution of the present data to re-examine the three-dimensional partitioning of the genome. In earlier experiments at 1 Mb map resolution, large squares of enhanced contact frequency tiling the diagonal of the contact matrices were seen. These squares partitioned the genome into 5-20 Mb intervals, which Applicants here call “megadomains.” On opposite sides of a megadomain boundary, the contact frequency between pairs of loci drops sharply. Megadomains are very frequently preserved across cell types.

It was also found that individual 1 Mb loci could be assigned to one of two long-range contact patterns, which are termed herein Compartments A and B, with loci in the same compartment showing more frequent interaction. Megadomains—and the associated squares along the diagonal—arise when all of the 1 Mb loci in an interval exhibit the same genome-wide contact pattern (Kalhor et al., Nature biotechnology 30, 90-98, 2012; Lieberman-Aiden et al., Science 326, 289-293, 2009; Sexton et al., Cell 148, 458-472, 2012). Compartment A is highly enriched for open chromatin, and correlates strongly with DNaseI accessibility, active genes, and H3K36me3. Compartment B is enriched for closed chromatin.

In the new, higher resolution maps presented herein, the inventors observed many small squares of enhanced contact frequency that tile the diagonal of each contact matrix (FIG. 4A). A dynamic programming algorithm was used to annotate these domains genome-wide. (Results using a previously published domain-calling algorithm (Dixon et al., 2012) were similar.) The observed domains range in size from 40 Kb to 3 Mb (median size 185 Kb). As with megadomains, there is an abrupt drop in contact frequency (33%) for pairs of loci on opposite sides of the domain boundary. Domains are very frequently preserved across cell type. The presence of smaller domains in Hi-C maps is consistent with other recent reports (Dixon et al., Nature 485, 376-380, 2012; Nora et al., Nature 485, 381-385, 2012; Sexton et al., Cell 148, 458-472, 2012), although the domains observed here are considerably smaller, likely due to the much larger dataset. Changes in histone marks at a domain are associated with changes in long-range contact pattern Loci within a domain show strongly correlated chromatin states for eight different histone modifications (H3K36me3, H3K27me3, H3K4me1, H3K4me2, H3K4me3, H3K9me3, H3K79me2, and H4K20me1) based on data from the ENCODE project in GM12878 cells (Consortium, 2011; Consortium et al., 2012). By contrast, loci at comparable distance but residing in different domains showed much less correlation in chromatin state (FIG. 4B). Strikingly, changes in a domain's chromatin state are often accompanied by changes in the long-range contact pattern of domain loci (i.e., the pattern of contacts between loci in the domain and other loci genome-wide), indicating that changes in chromatin pattern are accompanied by shifts in a domain's nuclear neighborhood (FIG. 2C, S25).

There are at Least Six Nuclear Subcompartments with Distinct Patterns of Histone Modifications.

Next, it was sought to characterize the long-range contact patterns in the data. Loci were partitioned into categories based on long-range contact patterns alone, using four independent approaches: manual annotation, and three objective clustering algorithms (HMM, K-means, Hierarchical). All gave similar results. The biological meaning of these categories was then investigated.

When the data was analyzed at low matrix resolution (1 Mb), the earlier finding of two compartments (A and B) was reproduced. At high resolution (25 Kb), however, strong evidence was found for at least five “subcompartments” defined by their long-range interaction patterns, both within and between chromosomes. The median length of an interval lying completely within a subcompartment was 300 Kb. Although the five subcompartments are defined solely based on their Hi-C interaction patterns, they show distinctive properties with respect to both their genomic and epigenomic content. Two of the five interaction patterns are strongly correlated with loci in compartment A. The loci exhibiting these patterns were labeled as belonging to subcompartments A1 and A2. Both A1 and A2 are gene dense, have highly expressed genes, harbor activating chromatin marks such as H3K36me3, H3K79me2, H3K27ac and H3K4me1 and are depleted at the nuclear envelope and at nucleolus associated domains (NADs). (See FIG. 2D,E) A2 is more strongly associated with the presence of H3K9me3 than A1, and the genes residing in A2 tend to be longer (2.4-fold). The other three interaction patterns (labeled B1, B2, and B3) are strongly correlated with loci in compartment B, and show very different properties. Subcompartment B1 correlates positively with H3K27me3 and negatively with H3K36me3, suggestive of facultative heterochromatin (FIG. 2D,E). Subcompartment B2 includes 62% of pericentromeric heterochromatin (3.8-fold enrichment) and is enriched at the nuclear envelope (1.8-fold) and at NADs (4.6-fold). Subcompartment B3 tends to lack all of the above-noted marks, suggesting ordinary heterochromatin; it is enriched at the nuclear envelope (1.6-fold), but strongly depleted at NADs (76-fold). (See FIG. 2D, S28A.) Upon closer visual examination, Applicants noticed the presence of a sixth pattern on chromosome 19 (FIG. 2F). The genome-wide clustering algorithm missed this pattern because it spans only 11 Mb, or 0.3% of the genome. When the algorithm was repeated on chromosome 19 alone, the additional pattern was detected. Because this sixth pattern correlates with the Compartment B pattern, it was labeled it B4. Subcompartment B4 comprises a handful of regions, each of which contain many KRAB-ZNF superfamily genes. (B4 contains 130 of the 278 KRAB-ZNF genes in the genome, a 65-fold enrichment). As noted in previous studies (Barski et al., Cell 129, 823-837, 2007; Hahn et al., PLoS One, 2011), these regions exhibit a distinctive chromatin pattern, with strong enrichment for both activating chromatin marks, such as H3K36me3, and heterochromatin-associated marks, such as H3K9me3 and H4K20me3.

In principle, the fact that domains lying in the same subcompartment exhibit similar chromatin marks might reflect either that (i) spatial proximity enhances the spread of histone modifications, or (ii) similarity of histone modifications helps bring about spatial proximity.

### Approximately 10,000 Peaks Mark the Position of Chromatin Loops

It was next sought to identify the positions of chromatin loops by using an algorithm to search for pairs of loci that show significantly closer proximity with one another than with the loci lying between them (FIG. 5A). Such pairs correspond to pixels with higher contact frequency than typical pixels in their neighborhood. These pixels are referred to as “peaks” in the Hi-C heatmap, and to the corresponding pair of loci as “peak loci”. Peaks reflect the presence of chromatin loops, with the peak loci being the anchor points of the chromatin loop. (Because contact frequencies vary across the genome, peak pixels are defined relative to the local background. Of Note, some papers have sought to define peaks relative to the genome-wide average. This choice is problematic because, for example, many pixels within a domain may be reported as peaks despite showing no locally distinctive proximity.). The algorithm detected 9448 peaks in the in situ Hi-C map for GM12878 at 5 kb map resolution. These peaks are associated with a total of 12,903 distinct peak loci (some peak loci are associated with more than one peak). The vast majority of peaks (98%) reflected loops between loci that are less than 2 Mb apart. (Examining the primary and replicate maps separately, 8054 peaks were found in the former and 7484 peaks in the latter, with 5403 in both lists. The differences were almost always the result of conservative peak-calling criteria.) As an independent confirmation that peak loci have greater physical proximity than neighboring locus pairs, 3D-FISH (Beliveau et al., Proceedings of the National Academy of Sciences of the United States of America 109, 21301-21306, 2012) was performed on 4 loops. In each case, two peak loci, L1 and L2, were compared with a control locus, L3, that lies an equal distance away from L2 but on the opposite side (FIG. 3C). In all cases, the distance between L1 and L2 was consistently shorter than the distance between L2 and L3. It was also confirmed that the list of peaks was consistent with previously published Hi-C maps. Although earlier maps contained too few contacts to reliably call individual peaks, the inventors developed a method called Aggregate Peak Analysis (APA) that compares the aggregate enrichment of the peak set in these low-resolution maps to the enrichment seen when the peaks are translated in any direction. APA showed strong consistency between the loop calls and all six previously published Hi-C datasets for lymphoblastoid cell lines (Kalhor et al., Nature biotechnology 30, 90-98, 2012; Lieberman-Aiden et al., Science 326, 289-293, 2009; FIG. 3D). Finally, it was demonstrated that the list of peaks was robust to particular protocol conditions by performing APA analysis on a GM12878 dilution Hi-C map, and on the 112 supplemental Hi-C experiments exploring a wide range of protocol variants. Enrichment was seen in every single experiment.

### Conservation of Peaks Among Human Cell Lines and Across Evolution

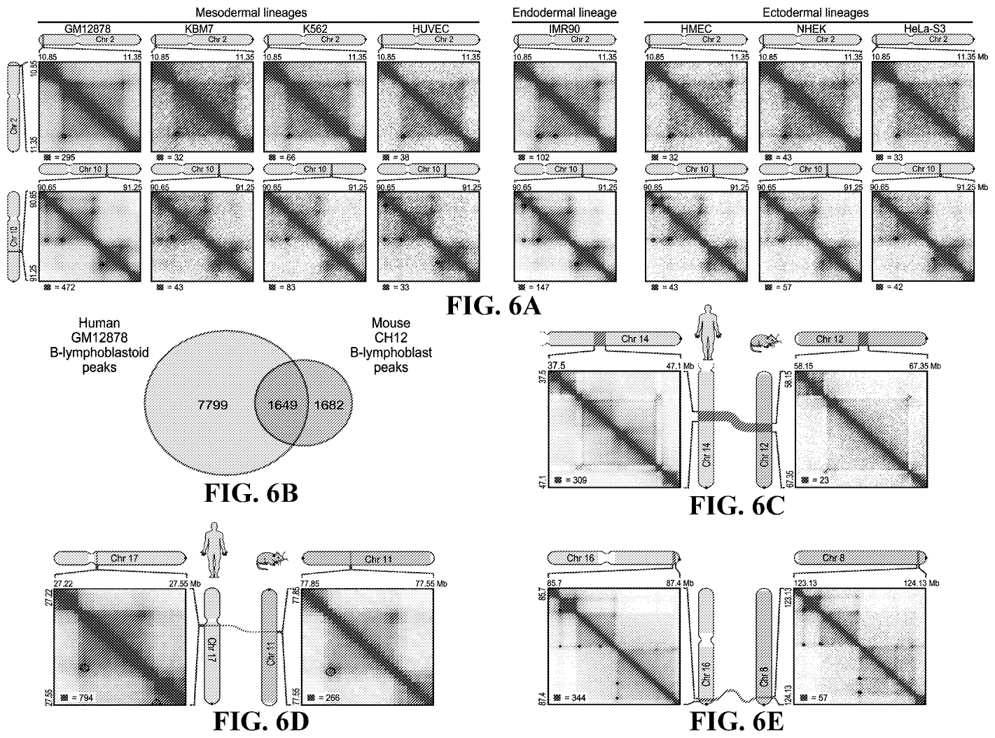

The inventors also identified peaks in the other six human cell lines (IMR90, HMEC, NHEK, K562, HUVEC, HeLa, and KBM7). Because these maps contain fewer contacts, sensitivity is reduced, and fewer peaks are observed (ranging from 2634 to 8040). Notably, APA analysis showed strong consistency between these peak calls and the dilution Hi-C maps reported here (in IMR90, HMEC, HUVEC, and NHEK), as well as with all previously published Hi-C maps in these cell types. Overall, it was found that peaks were strongly conserved across cell types (FIG. 6A): approximately half of the peaks found in any given cell type were also found in GM12878. Applicants also compared peaks across species. In CH12-LX mouse B-lymphoblasts, Applicants identified 2927 high-confidence domains and 3331 peaks. There was a strong correspondence between orthologous regions in GM12878 and CH12-LX. Overall, 50% of peaks and 45% of domains called in mouse were also called in humans, suggesting strong conservation of three-dimensional genome structure across the mammals (FIG. 6B-E).

Loops Anchored at a Promoter are Associated with Enhancers and Increased Gene Activation

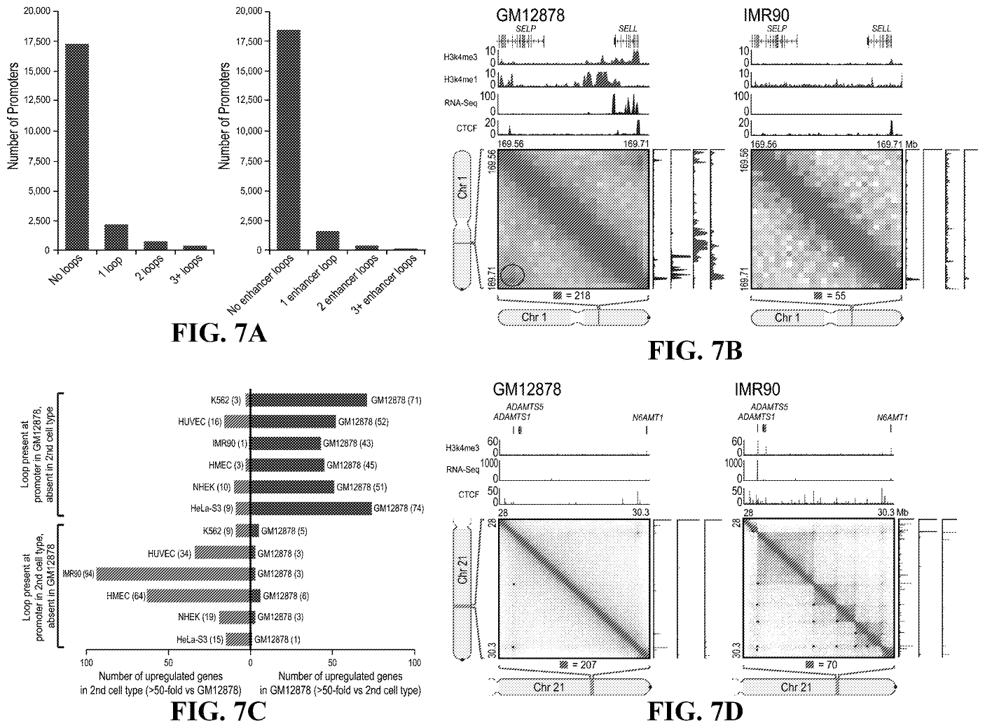

Various lines of evidence indicate that many of the observed loops, defined by the peaks, are associated with gene regulation. First, the peaks frequently have a known promoter at one peak locus (as annotated by ENCODE's ChromHMM), and a known enhancer at the other (FIG. 7A). For instance, 2854 of the 9448 peaks in Applicants' GM12878 map bring together known promoters and known enhancers (30%, vs. 7% expected by chance). These peaks include well-studied promoter-enhancer loops, such as at MYC (chr8:128.35-128.75 Mb) and alpha-globin (chr16:0.15-0.22 Mb). Second, genes whose promoters are associated with a loop are much more highly expressed (6-fold). Third, the presence of cell type-specific peaks is associated with changes in gene expression.

Although peaks are strongly correlated across cell types, there were also many cases in which a peak was present in one cell type but not another. When Applicants examined RNA-Seq data produced by ENCODE (ENCODE Consortium, 2011; ENCODE Consortium et al., 2012), it was found that the appearance of a loop in a cell type was frequently accompanied by the activation of a gene whose promoter overlapped one of the peak loci. For instance, 510 loops were observed in IMR90 that were clearly absent in GM12878. The corresponding peak loci overlapped the promoters of 94 genes that were markedly upregulated in IMR90 (>50-fold difference in RNA level), but of only 3 genes that were markedly upregulated in GM12878 (31-fold depletion). Conversely, 557 loops were found in GM12878 that were clearly absent in IMR90. The corresponding peak loci overlapped the promoters of 43 genes that were markedly upregulated in GM12878, but of only 1 gene that was markedly upregulated in IMR90: a 43-fold depletion. When GM12878 was compared to the five other human cell types for which ENCODE RNA-Seq data was available (all but KBM7), the results were very similar (FIG. 75B). One example of a cell-type specific loop is anchored at the promoter of the SELL gene, which encodes L-selectin, a lymphocyte-specific surface marker that is expressed in GM12878 but not IMR90 (FIG. 7C). Gene activation is occasionally accompanied by the emergence of a cell-type specific network of peaks. FIG. 7D illustrates the case of ADAMTS1, which encodes a protein involved in fibroblast migration. The gene is expressed in IMR90, where its promoter is involved in six loops. In GM12878, it is not expressed, and the promoter is involved in only two loops. Many of the IMR90 peak loci form transitive peaks with one another, suggesting that the ADAMTS1 promoter and the six distal sites may all be spatially co-located.

### Peaks Frequently Demarcate the Boundaries of Domains

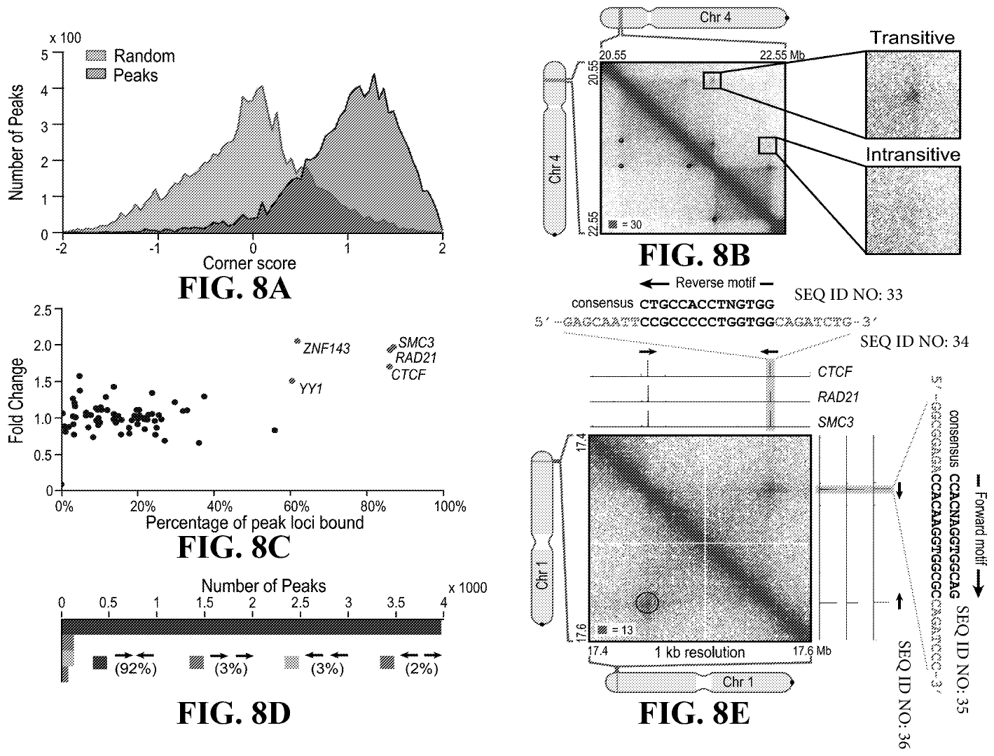

A large fraction of peaks (38%) coincide with the corners of a domain—that is, the peak loci are located at domain boundaries (FIG. 8A). Conversely, a large fraction of domains (39%) had peaks in their corner. Moreover, the appearance of a loop is usually (in 65% of cases) associated with the appearance of a domain demarcated by the loop. Because this configuration is so common, Applicants will use the term “loop domain” to refer to domains whose endpoints form a chromatin loop.

In some cases, adjacent loop domains (bounded by peak loci L1-L2 and L2-L3, respectively) exhibit transitivity—that is, L1 and L3 also correspond to a peak. In these situations, the three loci may simultaneously co-locate at a single spatial position. However, many peaks do not exhibit transitivity, suggesting that the loci may not co-locate simultaneously. FIG. 8B shows a region on chromosome 4 exhibiting both configurations. It was also found that overlapping loops are strongly disfavored: pairs of loops L1-L3 and L2-L4 (where L1, L2, L3 and L4 occur consecutively in the genome) are found far less often than expected under a random model.

The Vast Majority of Peaks are Associated with Pairs of CTCF Motifs in a Convergent Orientation

It was next asked whether peaks are associated with specific proteins. Applicants therefore examined the results of 86 ChIP-Seq experiments performed by ENCODE in GM12878 (ENCODE Consortium, 2011; ENCODE Consortium et al., 2012). Strikingly, it was found that the vast majority of peak loci are bound by the insulator protein CTCF (86%) and the cohesin subunits RAD21 (86%) and SMC3 (87%) (FIG. 8C). Indeed, most peak loci contain a unique DNA site containing a CTCF binding motif, to which all three proteins (CTCF, SMC3, and RAD21) were bound (5-fold enrichment). Applicants were thus able to associate most of the peak loci (6991 of 12,903) with a specific CTCF binding site “anchor”. The consensus DNA sequence for CTCF binding sites is typically written as 5′-CCACNAGGTGGCAG-3′ (SEQ ID NO: 20). Because the sequence is not palindromic, each CTCF site has an orientation; Applicants designate the consensus motif above as the ‘forward’ orientation. Thus, a pair of CTCF sites on the same chromosome can have four possible orientations: (1) same direction on one strand; (2) same direction on the other strand; (3) convergent on opposite strands; and (4) divergent on opposite strands. If CTCF sites were randomly oriented, one would expect all 4 orientations to occur equally often. But when Applicants examined the 4322 peaks in GM12878 where the two corresponding peak loci each contained a single CTCF binding motif, Applicants found a stunning result: the vast majority (92%) of motif pairs are convergent (FIG. 6D,E). Overall, the presence, at pairs of peak loci, of bound CTCF sites in the convergent orientation was enriched 102-fold over random expectation. Notably, the convergent orientation was overwhelmingly more frequent than the divergent orientation, despite the fact that divergent motifs also lie on opposing strands: in GM12878, the counts were 3971-78 (51-fold enrichment of convergent vs. divergent); in IMR90, 1456-5 (291-fold); in HMEC, 968-11 (88-fold); in K562, 723 to 2 (362-fold); in HUVEC, 671-4 (168-fold); in HeLa, 301-3 (100-fold); in NHEK, 556-9 (62-fold); and in CH12, 625-8 (78-fold). This surprising pattern suggests that a pair of CTCF sites in the convergent orientation is required for the formation of a loop. The observation that looped CTCF sites occur in the convergent orientation also allows Applicants to analyze peak loci containing multiple CTCF-bound motifs to predict which motif instance plays a role in a given loop. In this way, Applicants can associate nearly two-thirds of peak loci (8175 of 12,903, or 63.4%) with a single CTCF binding site. The specific orientation of CTCF sites at observed peaks provides strong evidence that Applicants' peak calls are biologically correct. Because randomly chosen CTCF pairs would exhibit each of the four orientations with equal probability, the near-perfect association between Applicants' loop calls and the particular orientation could not occur by chance (p<10-1900). In addition, the presence of CTCF and RAD21 sites at many of Applicants' peaks provides an opportunity to compare their results to three recent CHIA-PET experiments reported by the ENCODE consortium (in GM12878 and K562) in which ligation junctions bound to CTCF (resp. RAD21) were isolated and analyzed. Applicants found strong concordance with their results in all three cases.

### Diploid Hi-C Maps Reveals Homolog-Specific Features, Including Imprinting-Specific Loops and Massive Domains and Loops on the Inactive X-Chromosome

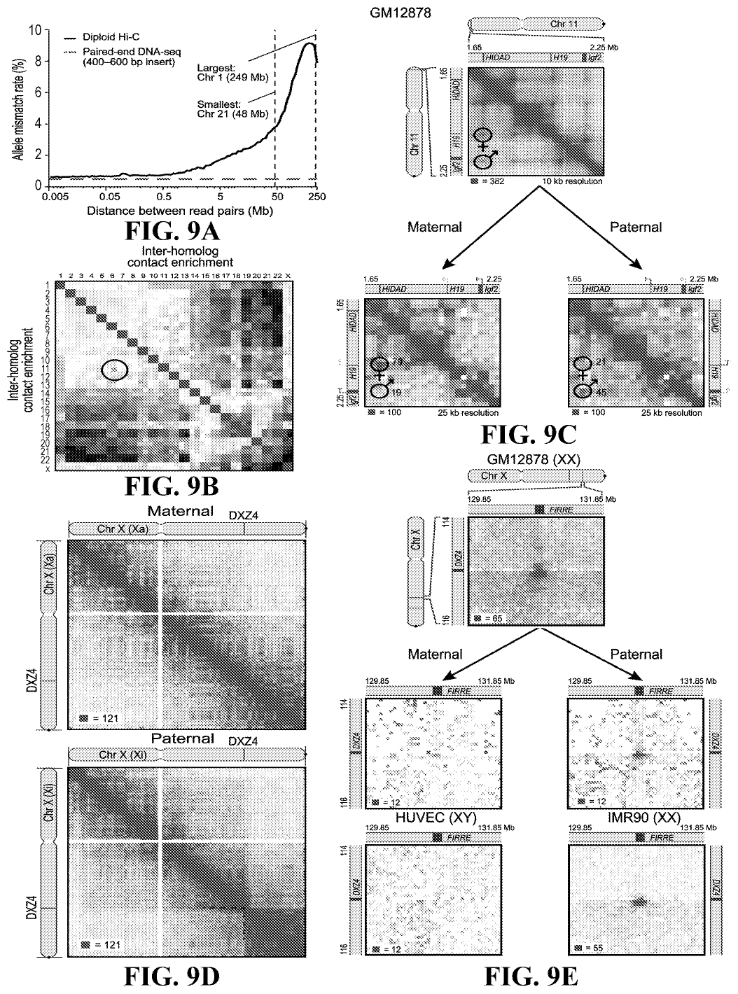

Because many of Applicants' reads overlap SNPs, it is possible to assign contacts to specific chromosomal homologs. Using GM12878 SNP-phasing data (Gil et al., Nature 491, 2012), Applicants found that they could frequently assign reads to either the maternal or paternal homolog (FIG. 9A). Using these assignments, Applicants constructed a “diploid” Hi-C map of GM12878 comprising both maternal (238M contacts) and paternal (240M) maps. Applicants studied these maps for differences between homologous chromosomes in contact frequencies, domain structure, and loop structure. For autosomes, the maternal and paternal homologs exhibit very similar inter- and intrachromosomal contact profiles (Pearson's R>0.998, P value negligible). One interchromosomal difference was notable: an elevated contact frequency between the paternal homologs of chromosome 6 and 11 that is consistent with an unbalanced translocation fusing chr11q:73.5 Mb and all distal loci (a stretch of over 60 Mb) to the telomere of chromosome 6p (FIG. 7B, S39). The signal intensity suggests that the translocation is present in between 1.2% and 5.6% of Applicants' cells. Applicants tested this prediction by karyotyping 100 GM12878 cells using Giemsa staining and found three abnormal chromosomes, each showing the predicted translocation, der(6)t(6,11)(pter;q) (FIG. S40-S41). Notably, the Hi-C data reveal that the translocation involves the paternal homologs, which cannot be determined with ordinary cytogenetic methods. Applicants also observed differences in loop structure between homologous autosomes at some imprinted loci. For instance, the H19/Igf2 locus on chromosome 11 is a well-characterized case of genomic imprinting. In their unphased maps, Applicants clearly see two loops from a single distal locus at 1.72 Mb (which binds CTCF in the forward orientation) to loci located near the promoters of both H19 and Igf2 (both of which bind CTCF in the reverse orientation, i.e., the above consensus motif lies on the opposite strand; see FIG. 7C). Applicants refer to this distal locus as the H19/Igf2 Distal Anchor Domain (HIDAD). Applicants' diploid maps reveal that the loop to the H19 region is present on the maternal chromosome (from which H19 is expressed), but the loop to the Igf2 region is absent or greatly attenuated. The opposite pattern is found on the paternal chromosome (from which Igf2 is expressed). Most strikingly, differences were seen on the diploid intrachromosomal maps of chromosome X. The paternal X chromosome, which is usually inactive in GM12878, is partitioned into two massive domains (0-115 Mb and 115-155.3 Mb). These “superdomains” are not seen in the active, maternal X (FIG. 7D). When Applicants examined the unphased maps of chromosome X for the karyotypically normal female cell lines in their study (GM12878, IMR90, HMEC, NHEK), the superdomains on X were evident, although the signal was markedly attenuated by the superposition of signals from active and inactive X chromosomes. When Applicants examined the male HUVEC cell line and the haploid KBM7 cell line, Applicants saw no evidence of superdomains (FIG. S42). Interestingly, the boundary between the superdomains (ChrX: 115 Mb+/−500 Kb) lies near the macrosatellite repeat DXZ4 (ChrX: 114,867,433-114,919,088) near the middle of Xq. DXZ4 is a CpG-rich tandem repeat that is conserved across primates and monkeys and encodes a long non-coding RNA. In males and on the active X, DXZ4 is heterochromatic, hyper-methylated and does not bind CTCF. On the inactive X, DXZ4 is euchromatic, hypo-methylated, and binds CTCF. DXZ4 has been hypothesized to play a role in reorganizing chromatin during X inactivation (Chadwick, 2008). There were also significant differences in loop structure between the chromosome X homologs. Applicants observed 27 extremely large “superloops,” each spanning between 7 and 74 Mb, present only on the inactive X chromosome in the diploid map (FIG. 7E). The superloops were also seen in all 4 unphased maps from karyotypically normal XX cells, but were absent in unphased maps from X0 and XY cells (FIG. S43). Two of the superloops (chrX:56.8 Mb-DXZ4 and DXZ4-130.9 Mb) have been reported previously, and their presence on the inactive X alone has been confirmed using multiple methods (Horakova et al., Human molecular genetics 21, 4367-4377, 2012). Like the peak loci of most other loops, nearly all the superloop anchors bind CTCF (25 of 26). The six anchor regions most frequently associated with superloops are very large (up to 200 kb). Four of these anchor regions contain whole lncRNA genes: loc550643; XIST; DXZ4; and FIRRE. Three (loc550643, and DXZ4, and FIRRE) contain CTCF-binding tandem repeats that only bind CTCF on the inactive homolog.

### DISCUSSION

The in situ Hi-C protocol allowed Applicants to probe genomic architecture with extremely high resolution; in the case of GM12878 lymphoblastoid cells, better than 1 kb. Applicants observe the presence of domains that were too small to be seen in Applicants' original Hi-C maps, which had resolution of 1 Mb (Lieberman-Aiden et al., Science 326, 289-293, 2009). Loci within a domain interact frequently with one another, have similar patterns of chromatin modifications, and exhibit similar long-range contact patterns. Domains tend to be conserved across cell types and between human and mouse. Strikingly, when the pattern of chromatin modifications associated with a domain changes, the domain's long-range contact pattern also changes. The domains exhibit six distinct patterns of long-range contacts (subcompartments), which subdivide the two compartments that Applicants had reported based on low resolution data. The subcompartments are each associated with distinct chromatin patterns. It is possible that the chromatin patterns play a role in bringing about the long-range contact patterns, or vice versa. High-resolution in situ Hi-C data makes it possible to create a genome-wide catalog of chromatin loops. Applicants identified loops by looking for pairs of loci that have significantly more contacts with one another than they do with other nearby loci. In their densest map, GM12878 lymphoblastoid cells, Applicants observe 9448 loops. Applicants note that their annotation identifies fewer loops than were reported in several recent high throughput studies. The key reason is that Applicants call peaks only when a pair of loci shows elevated contact frequency relative to the local background—that is, when the peak pixel is enriched as compared to other pixels in its neighborhood. In contrast, several previous studies have defined peaks by comparing the contact frequency at a pixel to the genome-wide average. This latter definition is problematic because many pixels within a domain can be annotated as peaks despite showing no local increase in contact frequency. Previous papers using the latter definition imply the existence of more than 100,000 or even more than 1 million peaks (Extended Experimental Procedures). The loops Applicants observe have many interesting properties. First, most loops are short (<2 Mb). Second, loops are strongly conserved across cell types and between human and mouse. Third, promoter-enhancer loops are common and are strongly associated with gene activation. Fourth, loops often demarcate domains, and may establish them. Fifth, loops tend not to overlap. Sixth, loops are closely associated with the presence of CTCF and the cohesin subunits RAD21 and SMC3; each of these proteins is found at over 86% of loop anchors. The most striking property of loops is that the pair of CTCF motifs present at the loop anchors occurs in a convergent orientation in >90% of cases (vs. 25% expected by chance). The importance of motif orientation between loci that are separated by, on average, 360 Kb is unexpected and must bear on the mechanism by which CTCF and cohesin form loops, which likely involves CTCF dimerization. Experiments in which the presence or orientation of CTCF sites is altered should shed light on this mechanism. Such experiments may also enable the engineering of loops, domains, and other chromatin structures.

Applicants also created diploid Hi-C maps, by using polymorphisms to assign contacts to distinct chromosomal homologs. Applicants find that the inactive X chromosome is partitioned into two large “superdomains” whose boundary lies near the locus of the lncRNA DXZ4 (Chadwick, 2008). Applicants also detect a network of extremely long-range (7-74 Mb) “superloops”, the strongest of which are anchored at locations containing lncRNA genes (loc550643, XIST, DXZ4, and FIRRE). With the exception of XIST, all of these lncRNAs contain CTCF-binding tandem repeats that bind CTCF only on the inactive X. Applicants hypothesize that Xi-specific CTCF binding participates in the formation of these massive chromatin structures. Just as loops bring distant DNA loci into close spatial proximity, Applicants find that they bring disparate aspects of DNA biology—domains, compartments, chromatin marks, and genetic regulation—into close conceptual proximity. As the understanding of the physical connections between DNA loci continues to improve, the understanding of the relationships between these broader phenomena will deepen.

### EXPERIMENTAL PROCEDURES

### In Situ Hi-C Protocol

All cell lines used were cultured following the manufacturer's recommendations. Cells were crosslinked with 1% formaldehyde for 10 minutes at room temperature. In situ Hi-C was performed by permeabilizing 2-5M nuclei. DNA was digested with 100 units of MboI (or DpnII), the ends of restriction fragments were labeled using biotinylated nucleotides, and were then ligated in a small volume. After reversal of crosslinks, ligated DNA was purified and sheared to a length of roughly 400 basepairs, at which point ligation junctions were pulled down with streptavidin beads and prepped for high-throughput Illumina® sequencing. Dilution Hi-C was performed as in (Lieberman-Aiden et al., Science 326, 289-293, 2009).

### 3D-FISH

FISH probes were designed using the OligoPaints database. DNA-FISH was performed as described in (Beliveau et al., Proceedings of the National Academy of Sciences of the United States of America 109, 21301-21306, 2012), with minor modifications.

### Hi-C Data Pipeline

All sequence data was produced using Illumina® paired-end sequencing. Sequence data was processed using a custom pipeline that was optimized for parallel computation on a cluster. The pipeline uses BWA (Li and Durbin, Bioinformatics (Oxford, England) 26, 589-595, 2010) to map each read end separately to the b37 or mm9 reference genomes; removes duplicate and near-duplicate reads; removes reads that map to the same fragment; and filters the remaining reads based on mapping quality score. Contact matrices were generated at base-pair delimited resolutions of 2.5 Mb, 1 Mb, 500 Kb, 250 Kb, 100 Kb, 50 Kb, 25 Kb, 10 Kb, and 5 Kb, as well as fragment-delimited resolutions of 500f, 200f, 100f, 50f, 20f, 5f, 2f, and 1f. For the largest data sets, the file also contains a 1 Kb contact matrix. Normalized contact matrices are produced at all resolutions using (Knight and Ruiz, IMA Journal of Numerical Analysis, 2012).

### Annotation of Domains

To annotate domains, a novel “arrowhead” transformation was applied, defined as Ai,i+d=(M*i,i−d−M*i,i+d)/(M*i,i−d+M*i,i+d). M* denotes the normalized contact matrix. This transformation can be thought of as equivalent to calculating a matrix equal to −1*(observed/expected−1), where the expected model controls for local background and distance from the diagonal in the simplest possible way: the “expected” value at i,i+d is simply the mean observed value at i,i−d and i,i+d. Ai,i+d will be strongly positive if and only if locus i−d is inside a domain and locus i+d is not. If the reverse is true, Ai,i+d will be strongly negative. If the loci are both inside or both outside a domain, Ai,i+d will be close to zero. Consequently, if there is a domain at [a,b], Applicants find that A takes on very negative values inside a triangle whose vertices lie at [a,a], [a,b], and [(a+b)/2,b], and very positive values inside a triangle whose vertices lie at [(a+b)/2,b], [b,b], and [b,2b-a]. The size and positioning of these triangles creates the arrowhead-shaped feature that replaces each domain in M*. A “corner score” matrix, indicating each pixel's likelihood of lying at the corner of a domain, is efficiently calculated from the arrowhead matrix using dynamic programming.

### Assigning Loci to Subcompartments

To cluster loci based on long-range contact patterns, Applicants constructed a 100 Kb resolution contact matrix comprising a subset of the interchromosomal contact data. Loci on odd chromosomes appeared on the rows, and loci from the even chromosomes appeared on the columns. (Chromosome X was excluded.) This matrix was clustered using the Python package scikit. To generate annotation of subcompartment B4, the 100 kb interchromosomal matrix for chromosome 19 was constructed and clustered separately, using the same procedure.

### Annotation of Peaks

The peak-calling algorithm examines each pixel in a Hi-C contact matrix and compares the number of contacts in the pixel to the number of contacts in a series of regions surrounding the pixel. The algorithm thus identifies pixels M*i,j where the contact frequency is higher than expected, and where this enrichment is not the result of a larger structural feature. For instance, ruling out the possibility that the enrichment of pixel M*i,j is the result of Li and Lj lying in the same domain by comparing the pixel's contact count to an expected model derived by examining the “lower-left” neighborhood. (The “lower-left” neighborhood samples pixels Mi′,j′ where i≤i′≤j′≤j; if a pixel is in a domain, these pixels will necessarily be in the same domain.) It is required that the pixel being tested contain at least 50% more contacts than expected, and that this enrichment be statistically significant after correcting for multiple hypothesis testing (FDR<10%). The same criteria are applied to three other neighborhoods. To be labeled an “enriched pixel,” a pixel must therefore be significantly enriched relative to four neighborhoods: (i) pixels to its lower-left; (ii) pixels to its left and right; (iii) pixels above and below; and (iv) a donut surrounding the pixel of interest (FIG. 6A). Using this approach, numerous enriched pixels were identified across the genome. The enriched pixels tend to form contiguous interaction regions comprising 5-20 pixels each. Applicants define the “peak pixel” (or simply the “peak”) to be the pixel in an interaction region with the largest number of contacts. Because over 10 billion (10 Kb)2 pixels must be examined, this calculation requires weeks of CPU time to execute. To accelerate it, a highly parallelized implementation was created using general-purpose graphical processing units, resulting in a 200-fold speedup relative to initial, CPU-based approach.

### Aggregate Peak Analysis

APA is performed on 10 Kb resolution contact matrices. To measure the aggregate enrichment of a set of putative peaks in a contact matrix, Applicants plot the sum of a series of submatrices derived from that contact matrix. Each of these submatrices is a 210 Kb×210 Kb square centered at a single putative peak in the upper triangle of the contact matrix. The resulting APA plot displays the total number of contacts that lie within the entire putative peak set at the center of the matrix; the entry immediately to the right of center corresponds to the total number of contacts in the pixel set obtained by shifting the peak set 10 Kb to the right; the entry two positions above center corresponds to an upward shift of 20 Kb, and so on. Focal enrichment across the peak set in aggregate manifests as larger values at the center of the APA plot. APA analyses only include peaks whose loci are at least 300 Kb apart.

### Example 2— Comparison of Results Obtained for In Situ Determination of Nucleic Acid Proximity as Described Herein and a Hi-C Protocol

As shown herein, the disclosed methods yield a result with greater complexity, which indicates more interactions that can be mapped and consequently more information. In other words, ‘complexity’ . . . this is the number of total contacts/datapoints produced by the experiment, thus the greater number of data points, the more information is extracted from each trial. In addition, method disclosed herein provide more the ‘large’ reads, which correspond to a long distance intrachromosomal contact. These contacts are the most informative ones, as they can pin down the long range interactions in the cell. The data presented herein demonstrate that the methods disclosed herein are superior than the previous Hi-C methods. The methods and protocols disclosed below are non-limiting examples of the methods disclosed herein and variation on the protocols in envisioned, such as the times, temperatures, and specific reagents used. Some steps may be omitted and others added.

### In Situ Hi-C Protocol Prepped for Illumina Sequencing

### Crosslinking

1) Grow two to five million cells under recommended culture conditions to about 80% confluence. Pellet suspension cells or detached adherent cells by centrifugation at 300×G for 5 min.
2) Resuspend cells in fresh medium at concentration of 1×10⁶ cells per 1 ml media. In a fume hood, add freshly made formaldehyde solution to a final concentration of 1%. Incubate at room temperature for 10 min with mixing. In some examples, no crosslinking is performed and the proximity relationships between nucleic acids are maintained via other means, for example by embedding nuclei in agarose.
3) Add 2.5M glycine solution to a final concentration of 0.2M to quench the reaction. Incubate at room temperature for 5 min on rocker.
4) Centrifuge for 5 min at 300×G at 4° C. Discard supernatant into an appropriate collection container.
5) Resuspend cells in 1 ml of cold 1×PBS and spin for 5 min at 300×G at 4° C. Discard supernatant and flash-freeze cell pellets in liquid nitrogen or dry ice/ethanol.
6) Either proceed to the rest of the protocol or store cell pellets at −80° C.

### Lysis and Restriction Digest

7) Combine 250 μl of ice-cold Hi-C lysis buffer (10 mM Tris-HCl pH8.0, 10 mM NaCl, 0.2% Igepal CA630) with 50 μl of protease inhibitors (Sigma, P8340). Add to one cross-linked pellet of cells.
8) Incubate cell suspension on ice for >15 minutes. Centrifuge at 2500×G for 5 minutes. Discard the supernatant.
9) Wash pelleted nuclei once with 500 μl of ice-cold Hi-C lysis buffer.
10) Gently resuspend pellet in 50 μl of 0.5% sodium dodecyl sulfate (SDS) and incubate at 62° C. for 5-10 minutes.
11) After heating is over, add 145 μl of water and 25 μl of 10% Triton® X-100 (Sigma, 93443) to quench SDS. Mix well, avoiding excessive foaming. Incubate at 37° C. for 15 minutes.
12) Add 25 μl of 10× NEBuffer2 and 100 U of MboI restriction enzyme (New England Biolabs (NEB, R0147)) and digest chromatin for at least 2 h or overnight at 37° C. with rotation.
In some examples, Hi-C can be performed with an additional centrifugation step added after restriction (step 12) and prior to fill-in.

### Marking of DNA Ends, Proximity Ligation, and Crosslink Reversal

13) Incubate at 62° C. for 20 minutes, then cool to room temperature.
14) To fill in the restriction fragment overhangs and mark the DNA ends with biotin, add 50 μl of fill-in master mix:
   37.5 μl of 0.4 mM biotin-14-dATP (Life Technologies, 19524-016)
   1.5 μl of 10 mM dCTP
   1.5 μl of 10 mM dGTP
   1.5 μl of 10 mM dTTP
   8 μl of 5 U/μl DNA Polymerase I, Large (Klenow) Fragment (NEB, M0210)
15) Mix by pipetting and incubate at 37° C. for 45 min-1.5 hours with rotation.
16) Add 900 μl of ligation master mix:
   663 μl of water
   120 μl of 10×NEB T4 DNA ligase buffer (NEB, B0202)
   100 μl of 10% Triton X-100
   12 μl of 10 mg/ml Bovine Serum Albumin (100XBSA)
   5 μl of 400 U/μl T4 DNA Ligase (NEB, M0202)
17) Mix by inverting and incubate at room temperature for 4 hours with slow rotation.
18) Degrade protein by adding 50 μl of 20 mg/ml proteinase K (NEB, P8102) and 120 μl of 10% SDS and incubate at 55° C. for 30 minutes.
   (In some examples nuclei can be pelleted after ligation (step 17) and then resuspended, both to remove random ligations that may have occurred in solution and to reduce the overall volume for ease of handling.)
19) Add 130 μl of 5M sodium chloride and incubate at 68° C. for at least 1.5 hours or overnight.

### DNA Shearing and Size Selection

20) Cool tubes at room temperature.
21) Split into two 750 μl aliquots in 2 ml tubes and add 1.6× volumes of pure ethanol and 0.1× volumes of 3M sodium acetate, pH 5.2, to each tube. Mix by inverting and incubate at −80° C. for 15 minutes.
22) Centrifuge at max speed, 2° C. for 15 minutes. Keeping tubes on ice after spinning, carefully remove the supernatant by pipetting.
23) Resuspend, combining the two aliquots, in 800 μl of 70% ethanol. Centrifuge at max speed for 5 minutes.
24) Remove all supernatant and wash the pellet once with 800 μl of 70% ethanol.
25) Dissolve pellet in 130 μl of 1× Tris buffer (10 mM Tris-Cl, pH 8) and incubate at 37° C. for 15 minutes to fully dissolve DNA.
26) To make the biotinylated DNA suitable for high-throughput sequencing using Illumina sequencers, shear to a size of 300-500 bp using the following parameters:
   Instrument: Covaris LE220 (Covaris, Woburn, Mass.)
   Volume of Library: 130 μl in a Covaris microTUBE
   Fill Level: 10
   Duty Cycle: 15
   PIP: 500
   Cycles/Burst: 200
   Time: 58 seconds
27) Transfer sheared DNA to a fresh 1.5 ml tube. Wash the Covaris vial with 70 μl of water and add to the sample, bringing the total reaction volume to 2000 Run a 1:5 dilution of DNA on a 2% agarose gel to verify successful shearing. For libraries containing fewer than 2×10⁶ cells, the size selection using AMPure XP beads described in the next steps could be performed on final amplicons rather than before pull-down.
28) Warm a bottle of AMPure XP beads (Beckman Coulter, A63881) to room temperature. To increase yield, AMPure XP beads can be concentrated by removing some of the clear solution before the beads are mixed for use in the next steps.
29) Add exactly 110 μl (0.55× volumes) of beads to the reaction. Mix well by pipetting and incubate at room temperature for 5 minutes.
30) Separate on a magnet. Transfer clear solution to a fresh tube, avoiding any beads. The supernatant will contain fragments shorter than 500 bp.
31) Add exactly 30 μl of fresh AMPure XP beads to the solution. Mix by pipetting and incubate at room temperature for 5 minutes.
32) Separate on a magnet and keep the beads. Fragments in the range of 300-500 bp will be retained on the beads.
33) Keeping the beads on the magnet, wash twice with 700 μl of 70% ethanol without mixing.
34) Leave the beads on the magnet for 5 minutes to allow remaining ethanol to evaporate.
35) To elute DNA, add 300 μl of 1× Tris buffer, gently mix by pipetting, incubate at room temperature for 5 minutes, separate on a magnet, and transfer the solution to a fresh 1.5 ml tube.
36) Quantify DNA by Qubit dsDNA High Sensitivity Assay (Life Technologies, Q32854) and run undiluted DNA on a 2% agarose gel to verify successful size selection.

### Biotin Pull-Down and Preparation for Illumina Sequencing

Perform all steps in low-bind tubes.
37) Prepare for biotin pull-down by washing 150 μl of 10 mg/ml Dynabeads MyOne Streptavidin T1 beads (Life technologies, 65602) with 400 μl of 1× Tween Washing Buffer (1×TWB: 5 mM Tris-HCl (pH 7.5); 0.5 mM EDTA; 1M NaCl; 0.05% Tween 20). Separate on a magnet and discard the solution.
38) Resuspend the beads in 300 μl of 2× Binding Buffer (2×BB: 10 mM Tris-HCl (pH 7.5); 1 mM EDTA; 2M NaCl) and add to the reaction. Incubate at room temperature for 15 minutes with rotation to bind biotinylated DNA to the streptavidin beads.
39) Separate on a magnet and discard the solution.
40) Wash the beads by adding 600 μl of 1×TWB and transferring the mixture to a new tube. Heat the tubes on Thermomixer at 55° C. for 2 min with mixing. Reclaim the beads using a magnet. Discard supernatant.
41) Repeat wash.
42) Resuspend beads in 100 μl 1×NEB T4 DNA ligase buffer (NEB, B0202) and transfer to a new tube. Reclaim beads and discard the buffer.
43) To repair ends of sheared DNA and remove biotin from unligated ends, resuspend in 100 μl of master mix:
   88 μl of 1×NEB T4 DNA ligase buffer with 10 mM ATP
   2 μl of 25 mM dNTP mix
   5 μl of 10 U/μl NEB T4 PNK (NEB, M0201)
   4 μl of 3 U/μl NEB T4 DNA polymerase I (NEB, M0203)
   1 μl of 5 U/μl NEB Klenow fragment of DNA polymerase I (NEB, M0210)
44) Incubate at room temperature for 30 minutes. Separate on a magnet and discard the solution.
45) Wash the beads by adding 600 μl of 1×TWB and transferring the mixture to a new tube. Heat the tubes on Thermomixer at 55° C. for 2 min with mixing. Reclaim the beads using a magnet. Discard supernatant.
46) Repeat wash.
47) Resuspend beads in 100 μl 1× NEBuffer 2 and transfer to a new tube. Reclaim beads and discard the buffer.
48) Resuspend in 100 μl of dATP attachment master mix:
   90 μl of 1× NEBuffer 2
   5 μl of 10 mM dATP
   5 μl of 5 U/μl NEB Klenow exo minus (NEB, M0212)
49) Incubate at 37° C. for 30 minutes. Separate on a magnet and discard the solution.
50) Wash the beads by adding 600 μl of 1×TWB and transferring the mixture to a new tube. Heat the tubes on Thermomixer at 55° C. for 2 min with mixing. Reclaim the beads using a magnet. Discard supernatant.
51) Repeat wash.
52) Resuspend beads in 100 μl 1× Quick ligation reaction buffer (NEB, B6058) and transfer to a new tube. Reclaim beads and discard the buffer.
53) Resuspend in 50 μl of 1×NEB Quick ligation reaction buffer.
54) Add 2 μl of NEB DNA Quick ligase (NEB, M2200). Add 3 μl of an Illumina indexed adapter. Record the sample-index combination. Mix thoroughly.
55) Incubate at room temperature for 15 minutes. Separate on a magnet and discard the solution.
56) Wash the beads by adding 600 μl of 1×TWB and transferring the mixture to a new tube. Heat the tubes on Thermomixer at 55° C. for 2 min with mixing. Reclaim the beads using a magnet. Remove supernatant.
57) Repeat wash.
58) Resuspend beads in 100 μl 1× Tris buffer and transfer to a new tube. Reclaim beads and discard the buffer.
59) Resuspend in 50 μl of 1× Tris buffer.

### Final Amplification and Purification

60) Amplify the Hi-C library directly off of the T1 beads with 4-12 cycles, using Illumina primers and protocol. In some examples to avoid PCR inhibition, one can detach DNA from the streptavidin beads by heating at 98 C. for 10 minutes after step 59 and then removing the beads with a magnet.)
61) After amplification is complete, bring the total library volume to 250 μl
62) Separate on a magnet. Transfer the solution to a fresh tube and discard the beads.
63) Warm a bottle of AMPure XP beads to room temperature. Gently shake to resuspend the magnetic beads. Add 175 μl of beads to the PCR reaction (0.7× volumes). Mix by pipetting and incubate at room temperature for 5 minutes.
64) Separate on a magnet and remove the clear solution.
65) Keeping the beads on the magnet, wash once with 700 μl of 70% ethanol without mixing.
66) Remove ethanol completely. To remove traces of short products, resuspend in 100 μl of 1× Tris buffer and add 70 μl more of AMPure XP beads. Mix by pipetting and incubate at room temperature for 5 minutes.
67) Separate on a magnet and remove the clear solution.
68) Keeping the beads on the magnet, wash twice with 700 μl of 70% ethanol without mixing.
69) Leave the beads on the magnet for 5 minutes to allow remaining ethanol to evaporate.
70) Add 25-50 μl of 1× Tris buffer to elute DNA. Mix by pipetting, incubate at room temperature for 5 minutes, separate on a magnet, and transfer the solution to a freshly labeled tube. The result is a final in situ Hi-C library ready to be quantified and sequenced using an Illumina sequencing platform.

### In Situ Hi-C can be Performed on Cells Embedded in Agar Plugs as Follows:

After lysis (above protocol, step 11), nuclei can be resuspended in 100 μl 2× NEBuffer2 and mixed with 100 μl molten 2% NuSieve agarose (Lonza, 5009) and allowed to solidify into an agarose plug. The nuclei embedded in agar are restricted overnight in 500 μl 1× NEBuffer2 with 100 U of MboI at 37° C.

After restriction, the buffer is discarded and the agar plug is washed twice with 1 ml of 1×NEB T4 DNA ligase buffer for 30 min at 37° C. The buffer is discarded and the agar plug is submerged in 0.5 ml fill-in reaction mix:
398 μl of water
50 μl of 10×NEB T4 DNA ligase buffer
37.5 μl of 0.4 mM biotin-14-dATP
1.5 μl of 10 mM dCTP
1.5 μl of 10 mM dGTP
1.5 μl of 10 mM dTTP
10 μl of 5 U/μl DNA Polymerase I, Large (Klenow) Fragment
   The library is incubated for 1.5 hours at room temperature. After incubation, 2000 U of T4 DNA Ligase are added to the reaction and the library is ligated at room temperature for 4 hours.

After ligation, the buffer is discarded and the agar plug is washed twice with 1 ml of 1×NEB β-agarase I buffer (NEB, B0392) for 30 min at 37° C. The buffer is removed and the agarose is melted by incubation at 68° C. for 10 minutes. Liquid agarose is equilibrated at 42° C. for 15 minutes. The agarose was digested with 4 U of β-Agarase I (NEB, M0392) at 42° C. for 1 hour. Next, the crosslinks can be reversed and all subsequent steps are performed following the standard in situ Hi-C protocol beginning at step 18.

### In Situ Determination of Nucleic Acid Proximity as Determined by the Inventors for Cell Line GM12878.

Library complexity: 5,013,218,921

### Inter: 26,989,930 (21.29%)

### Intra: 99,786,882 (78.71%)

### Small: 28,929,777 (22.82%)

### Large: 70,857,049 (55.89%)

### In Situ Determination of Nucleic Acid Proximity as Determined by the Inventors for Cell Line IMR-90.

### Library Complexity: 4,539,616,093

### Inter: 23,982,997 (19.20%)

### Intra: 100,952,857 (80.80%)

### Small: 25,712,979 (20.58%)

### Large: 75,237,444 (60.22%)

Hi-C Methodology as Described in McCord et al., Genome Res. Vol. 23 No. 2, Pp 260-269, 2013, which is Specifically Incorporated Herein by Reference in its Entirety (See Example 3)
Library complexity: 601,980,531

### Inter: 11,681,267 (22.38%)

### Intra: 40,503,943 (77.62%)

### Small: 34,209,456 (65.55%)

### Large: 6,292,643 (12.06%)

Hi-C Methodology as Described in Rickman et al., PNAS, USA, Vol. 109 No. 23, Pp 9083-9088, 2012, which is Specifically Incorporated Herein by Reference in its Entirety (See Example 4).
Library complexity: 107,614,087

### Inter: 17,204,445 (36.84%)

### Intra: 29,500,589 (63.16%)

### Small: 17,708,289 (37.92%)

### Large: 11,783,647 (25.23%)

### Example 3—Analysis of Human Fibroblasts Using Hi-C

This example describes the analysis of human fibroblasts using the Hi-C methodology as described in McCord et al., Genome Res. Vol. 23 no. 2, pp 260-269, 2013.

### Cell Lines

The three primary fibroblast cell lines used in the Hi-C experiments were HGADFN167 (HGPS), HGFDFN168 (Father, normal), and AG08470 (Age control, normal). Additional fibroblast lines were used in EZH2 RT-qPCR analysis, and these cell lines were HGADFN169 (HGPS), HGADFN164 (HGPS), HGADFN155 (HGPS), and HGFDFN090 (normal). AG08470 was obtained from Coriell, and the other cell lines were obtained from the Progeria Research Foundation. These primary human dermal fibroblasts were cultured in MEM (Invitrogen/GIBCO) supplemented with 15% fetal bovine serum (FBS) (Invitrogen) and 2 mM L-glutamine.

### Hi-C Library Preparation

20 million cells from an HGPS cell line (HGADFN167) at two increasing passages (p17 and 19), as well as from two normal fibroblast cell lines at similar passages (HGFDFN168-p18 and AG08470-p20) were crosslinked in 1% formaldehyde. HGFDFN168-p18 is the father of the HGPS patient HGADFN167, and AG08470 is an age matched, unrelated child. Hi-C was performed essentially as described previously (Lieberman-Aiden et al. 2009). Cells were lysed, and chromatin was digested with HindIII. Digested ends were filled in with biotinylated dCTP and then ligated for 4 hours at 16 C. After reversing the formaldehyde crosslinks by incubation at 65 C. with Proteinase K overnight and removing unligated biotinylated ends with T4 DNA polymerase, the DNA was fragmented by Covaris sonication to an average size of 200 bp and then the ideal size for Illumina sequencing (100-300 bp) was selected by Ampure fractionation. The DNA ends were repaired and ‘A’-tailed and then biotinylated junctions were pulled down using MyOne streptavidin beads. Illumina paired end adapters were ligated onto the DNA ends and then the fragments were PCR amplified for the minimum number of cycles necessary to generate 10 nM final DNA concentration.

### Hi-C Data Processing

Samples were sequenced on an Illumina GAIT instrument using the Paired End 75 bp module. Sequencing reads from the Hi-C experiment were mapped to the hg18 genome using Bowtie2 using the “very-sensitive” settings in an iterative procedure as follows: first, the 5′ 25 bp of each sequence was mapped, and then any reads that were unmapped or not mapped uniquely were extended to 30 bp, then 35 bp, etc. until the maximum length of the sequence was reached. This procedure aids in mapping sequences that read through a ligation junction near their 3′ end and whose full-length sequence would thus be unmappable. Aligned reads were assigned to restriction fragments and filtered to discard duplicate read pairs (PCR over-amplification products) and molecules for which both ends map to the same restriction fragment.

Restriction fragments shorter than 100 bp or longer than 100 kb as well as those with the top 0.5% of read counts were removed. After these filtering steps, 10-20 million valid interaction pairs were obtained for each sample. Reads were assigned to genomic bins of 200 kb, according to the center of their corresponding restriction fragment. The binned interaction maps were then corrected for systematic biases by equalizing the total coverage (1D sum across the matrix) of every bin in the genome using 50 iterations of a normalization procedure previously described (Imakaev et al. 2012; Zhang et al. 2012). The final data was then smoothed with a 1 Mb bin size and 200 kb step size.

### Hi-C Data Analysis and Comparison to Other Datasets

Open and closed chromatin compartments were identified as previously described (Lieberman-Aiden et al. 2009). Briefly, the expected number of Hi-C reads between bins separated by each genomic distance was calculated using a loess-smoothed average over the dataset. The log ratio of observed Hi-C reads to this expected value was then calculated. The Pearson correlation between the patterns of chromosomal interactions at each pair of bins was then calculated, and this correlation matrix was used to perform Principal Components Analysis. The eigenvector of the first principal component was then plotted as the compartment assignment, with positive values corresponding to regions of high gene density (“compartment A” or “open chromatin”) and negative values corresponding to regions of low gene density (“compartment B” or “closed chromatin”). The gene density was determined by calculating the number of genes in each bin according to the UCSC Known Canonical table of human genes.

### Example 4— Analysis of Human Fibroblasts Using Hi-C

This example describes the analysis of RWPE1-ERG and RWPE1-GFP cell lines.

### Human Cell Lines.

RWPE1 and DU145 cells were obtained from ATCC and maintained according to the manufacturer's protocol using isogenic cell lines overexpress either truncated ERG (most commonly encoded isoform based on TMPRSS2-ERG fusion).

### Hi-C Library Generation.

Fifty million RWPE1-ERG or RWPE1-GFP cells were fixed and processed to generate Hi-C libraries. Briefly, cells were cross-linked and the chromatin was digested with HindIII, ligated after fill-in with biotin-conjugated dCTP, and purified using streptavidivin-conjugated magnetic beads. The Hi-C libraries were then paired-end sequenced using an Illumina GAIIx platform, resulting in replicate-combined 158.5 million and 159.2 million paired-end DNA sequence reads from RWPE1-ERG and RWPE1-GFP, respectively.

### Hi-C

Fifty million RWPE1-ERG or RWPE1-GFP cells were fixed and processed to generate Hi-C libraries as previously reported. Briefly, cells were cross-linked, and the chromatin was digested with HindIII, ligated after fill-in with biotin-conjugated dCTP, and purified using streptavidivin-conjugated magnetic beads.

### SI Computational Analysis

Sequence Alignment and Extraction of Hi-C Interactions. Applicants aligned the two ends of the 54-bp paired reads separately to the reference human genome hg18 (NCBI build 36), using the BWA aligner.

Reads mapped ambiguously to multiple locations on the genome were discarded. Applicants further filtered out clonal reads caused by PCR artifacts on the basis of the 5′ and 3′ read positions, removed nonligated DNA fragments, and retained ones with consistent expected placement relative to HindIII enzyme digestion sites. In total, Applicants obtained more than 32 million intra- and interchromosomal interactions in each cell line.

### Example 5— Hybrid Capture Hi-C

As implemented in this Example, the disclosed example embodiment involves generating a probe set to detect target ligation junctions, the probes in the probe set comprising one or more labeled nucleotides. The probes in the probe set are designed to target sequences within a certain distance of known restriction sites in the genome to be analyzed. Ligation junctions are formed as described previously with the exception that labeled nucleotides do not have to be incorporated to fill in the overhanging fragmented ends. The generated probe set is allowed to hybridize to the formed ligation junctions and the one or more labeled nucleotides in the hybridized probed are then used to isolate the one or more end joined nucleic fragments (junctions). To determine the sequence of the target junction is then determined using nucleic acid sequencing.

i. Probe Design

To design probes targeting a particular region for HYbrid Capture Hi-C(Hi-C2), all restriction sites within the target region were identified. Since Hi-C ligation junctions occur between restriction sites, bait probe sequences were designed to target sequences within a certain distance of the identified restriction sites present in the target region. In this particular embodiment MboI restrictions sites were used. Specifically, a first pass was performed scanning all 120 bp sequences with one end within 80 bp of a restriction site and selecting, for each restriction end (i.e., both upstream and downstream of the restriction site), the closest 120 bp sequence to the restriction site that had fewer than 10 repetitive bases (as determined by the repeat masked hg19 genome downloaded from UCSC) and had between 50% and 60% GC content. If there was no probe satisfying those criteria, the closest probe with between 40% and 70% GC content but satisfying all the other above criteria was retained. The GC content bounds were chosen based on the hybridization bias data known in the art.

After the first pass, one probe from any pair of probes that overlapped was removed. Gaps in the probe coverage were identified, for example intervals larger than 110 bp, and any restriction sites falling within those gaps identified. Additional 120 bp probes were then searched using the following relaxed set of criteria. For each restriction site within a gap, all 120 bp sequences with one end within 110 bp of a restriction site were scanned and the closest sequence to the restriction site that had fewer than 20 repetitive bases and had between 40 and 70% GC content was selected. After the second pass, gaps in the probe coverage of at least 110 bp were identified. For gaps that fell within 5 kb windows in the target region that were covered by fewer than 5 probes, a third probe design pass was performed. For each restriction site within these low coverage gaps, all 120 bp sequences with one end within 110 bp of a restriction site were scanned and the closest sequence to the restriction site that had fewer than 25 repetitive bases and had between 25% and 80% GC content was selected.

ii. Probe Construction

Custom synthesized pools of 150 bp (120 bp+15 bp primer sequence on either end) single stranded oligodeoxynucleotides were obtained from CustomArray, Inc. (Bothell, Wash.). The oligonucleotides were of the general form TCGCGCCCATAACTCN₁₂₀CTGAGGGTCCGCCTT (SEQ ID NO: 21) for Region 1, ATCGCACCAGCGTGTN₁₂₀CACTGCGGCTCCTCA (SEQ ID NO: 22) for Region 2, and CCTCGCCTATCCCATN₁₂₀CACTACCGGGGTCTG (SEQ ID NO: 23) for Region 3. Region-specific sub-pools were first amplified from the overall CustomArray oligo pool using the following mix and PCR profile:

| | |
|---|---|
| 2 ul | oligo pool (160 ng) |
| 6 ul | Primer 1 (10 uM) |
| 6 ul | Primer 2 (10 uM) |
| 36 ul | H2O |
| 50 ul | 2X Phusion master mix |
| 100 ul | TOTAL |

Amplify for 10-18 cycles using the following PCR profile:
   98 C. for 30 s
      98 C. for 10 s
      55 C. for 30 s
   72 C. for 30 s cycle 10-18 times
   72 for 7 min
   hold at 4 C.
      where Primer 1 was CTGGGATCGCGCCCATAACTC (SEQ ID NO: 24) for Region 1, CTGGGAATCGCACCAGCGTGT (SEQ ID NO: 25) for Region 2, CTGGGACCTCGCCTATCCCAT (SEQ ID NO: 26) for Region 3 and Primer 2 was CGTGGAAAGGCGGACCCTCAG (SEQ ID NO: 27) for Region 1, CGTGGATGAGGAGCCGCAGTG (SEQ ID NO: 28) for Region 2, CGTGGACAGACCCCGGTAGTG (SEQ ID NO: 29) for Region 3.

After the initial amplification of the region-specific sub-pool, a 1×SPRI clean up was performed on the 162 bp PCR product to remove primers and primer-dimers. Applicants then performed a second PCR amplification to add a T7 promoter, using the following mix and PCR profile:

| | |
|---|---|
| 2 ul | first PCR product |
| 12 ul | Primer 1-T7 (10 uM) |
| 12 ul | Primer 2 (10 uM) |
| 74 ul | H2O |
| 100 ul | 2X Phusion master mix |
| 200 ul | TOTAL |

Amplify for 12-18 cycles using the following PCR profile:
   98 C. for 30 s
      98 C. for 10 s
      55 C. for 30 s
      72 C. for 30 s cycle 12-18 times
72 for 7 min
hold at 4 C.

where Primer 1—T7 was

(SEQ ID NO: 30)
GGATTCTAATACGACTCACTATAGGGTCGCGCCCATAACTC for
Region 1,
(SEQ ID NO: 31)
GGATTCTAATACGACTCACTATAGGGATCGCACCAGCGTGT for
Region 2,
and
(SEQ ID NO: 32)
GGATTCTAATACGACTCACTATAGGGCCTCGCCTATCCCA for
Region 3.

After the second PCR, once again, a 1×SPRI clean up to purify the 182 bp PCR product was performed. The purified second PCR product was then used as the template in a MAXIScript T7 transcription reaction (Ambion) as follows:

| | |
|---|---|
| Xul | purified DNA template (1 ug) |
| 10 ul | T7 enzyme mix |
| 10 ul | 10X transcription buffer |
| 5 ul | 10 mMATP |
| 5 ul | 10 mMCTP |
| 5 ul | 10 mMGTP |
| 4 ul | 10 mMUTP |
| 1 ul | 10 mM Biotin-16-UTP |
| Yul | H2O |
| 100 ul | TOTAL |

After incubating the reaction for at least 90 minutes at 37 C., 1 ul of TURBO DNase 1 was added and incubated at 37 C.° for 15 minutes to remove template DNA. An aliquot of 1 ul of 0.5M EDTA was added to stop the reaction and unincorporated nucleotides were removed and the RNA desalted by purifying the RNA probes using a Zymo Oligo Clean and Concentrator column (following manufacturer's instructions). The RNA yield was typically 5-15 ug of RNA per reaction, so the concentration of the RNA prior to the column cleanup using a Qubit RNA assay was measured in order to determine whether to use one or two columns (the capacity of one of the Zymo columns is 10 ug). For long-term storage of the RNA probes, 1 U/ul of SUPERase-In RNase inhibitor (Ambion) was added and the probes were stored at −80 C.

iii. Hybrid Selection

Final in situ Hi-C libraries were assessed for quality using the metrics outlined in Rao et al. Cell. 2014 159(7):1665-80. High quality libraries of sufficient complexity were selected for hybrid capture. 500 ng of Hi-C library was used as the pond for the hybrid selection reaction; libraries were diluted to a concentration of 20 ng/ul (i.e. 25 ul of library was used). For a few libraries that were under 20 ng/ul in concentration, as low as 250 ng total was used (still in 25 ul).

For the hybridization reaction, 25 ul of pond was mixed with 2.5 ug (1 ul) of Cot-1 DNA (Invitrogen) and bug (1 ul) of salmon sperm DNA (Stratagene). The DNA mixture was heated to 95 C. for 5 minutes and then held at 65 C. for at least 5 minutes. After at least 5 minutes at 65 C., 33 ul of prewarmed (65 C.) hybridization buffer (10×SSPE, 10×Denhardt's buffer, 10 mM EDTA, and 0.2% SDS) and 6 ul of RNA probe mixture (500 ng of RNA probes, 20 U of SUPERase-In RNase inhibitor; prewarmed at 65 C. for 2 minutes) were added to the DNA library for a total volume of −66 ul. This mixture was incubated at 65 C. in a thermocycler for 24 hours.

After 24 hours at 65 C., 50 ul of streptavidin beads (Dynabeads MyOne Streptavidin T1, Life Technologies) were washed three times in 200 ul of Bind-and-Wash buffer (1M NaCl, 10 mM Tris-HCl, pH 7.5, and 1 mM EDTA) and then resuspended in 134 ul of Bind-and-Wash buffer. The beads were added to the hybridization mixture and incubated for 30 minutes at room temperature (with occasional mixing to prevent the beads from settling). After 30 minutes, the beads were separated with a magnet and the supernatant discarded. The beads were then washed once with 200 ul low-stringency wash buffer (1×SSC, 0.1% SDS) and incubated for 15 minutes at room temperature. After 15 minutes, the beads were separated on a magnet and the supernatant discarded. The beads were then washed three times in high-stringency wash buffer (0.1×SSC, 0.1% SDS) at 65 C. for 10 minutes, each time separating the beads with a magnet and discarding the supernatant.

After the last wash, the DNA was eluted off the beads by resuspending in 50 ul of 0.1M NaOH and incubating for 10 minutes at room temperature. After 10 minutes, the beads were separated on a magnet and the supernatant was transferred to a fresh tube with 50 ul of 1M Tris-HCl, pH 7.5 (to neutralize the NaOH).

To desalt the DNA, Applicants performed a 1×SPRI cleanup using 3× concentrated SPRI beads (taking 3 volumes of SPRI bead/solution mix, separating on a magnet, discarding 2 volumes of SPRI solution and resuspending the beads in the remaining 1 volume). Applicants eluted the DNA in 22.5 ul of 1×Tris buffer (10 mM Tris-HCl, pH 8.0).

In order to prep the Hi-C² library for sequencing, Applicants added 25 ul of 2× Phusion and 2.5 ul of Illumina primers and amplified the library for 12-18 cycles. After PCR, Applicants performed two 0.7×SPRI cleanups to remove primers, etc. and then quantified the libraries for sequencing.

iv. Hi-C² Data Processing

Hi-C² libraries were sequenced to a depth of between −600K-60M reads (on average, 7.8M reads). All data was initially processed using the pipeline published in Rao et al. (2014). However, additional processing was needed to properly normalize the Hi-C2 data.

Normalization is an important problem to address in the analysis and interpretation of all proximity ligation experiments. It was previously shown that matrix balancing with the KR algorithm is an effective tool for properly normalizing Hi-C data (Rao and Huntley, et al. Cell 2014). However, one requirement of the KR algorithm is the requirement of a square symmetric matrix. As hybrid selection strongly enriches for certain rows of the matrix corresponding to the target region, there are large regions of the overall matrix that are extremely sparse (entries corresponding to interactions between two non-target loci). As a result, performing KR matrix balancing on the overall matrix generated by a Hi-C2 experiment does not efficiently correct both first-order hybrid selection target-enrichment biases and second-order hybridization biases within the target region.

To deal with this, a previously generated high-resolution genome-wide in situ Hi-C map of wild-type of Hap1 was used to normalize the data. Since all genome-editing perturbations were made within the region targeted using Hi-C2, for every Hi-C2 dataset, data from the genome-wide wild-type Hap1 map corresponding to regions of the chromosome-wide matrix where both loci fall outside of the target region were spiked in. Spiked data was added such that the average coverage of a locus in the overall chromosome-wide matrix was equal to the average coverage of loci within the target region. By spiking in data from the wild-type map where expectation is to see no change (since there were no perturbations), the first-order bias from hybrid-selection target enrichment could be removed, and KR matrix balancing used on the entire chromosome-wide matrix (which is no longer extremely sparse) to correct the second-order hybridization biases. Several different flavors of this normalization scheme may be implemented yielding extremely similar results; they are described below. The example methods described below may be used to normalize the data.
a. Raw gap-filling: For a given resolution, the average intrachromosomal coverage of the loci within the target region (defined as the entire interval tiled by probes not specifically the loci that were covered by a probe) was calculated from the raw uncorrected Hi-C² matrix. Similarly, the average intrachromosomal coverage of all loci was calculated from the raw uncorrected genome-wide Hap1 wild-type Hi-C map. A matrix consisting of all entries corresponding to two loci that were both outside the target region was constructed from the raw uncorrected genome-wide Hap1 Hi-C map. This matrix was multiplied by the ratio of the average coverage of loci within the target region in the Hi-C² data to the average coverage of all loci from the genome-wide Hap1 wild-type Hi-C data and then summed with the Hi-C² matrix (thereby filling in the extremely sparse areas of the Hi-C² matrix). This summed matrix was then corrected with the KR matrix balancing algorithm. The resulting normalization factors were used as correction factors for the Hi-C² data.
b. KR gap-filling: The KR gap-filling normalization was performed similarly to the method described above, but to avoid corrected Hi-C biases and Hi-C² biases together, the method above was performed on KR normalized data. Specifically, the KR correction factors derived from the genome-wide Hap1 wild-type Hi-C map were used to perform an initial correction of the Hi-C² data. After the initial correction, the average intrachromosomal coverage of the loci within the target region (defined as the entire interval tiled by probes not specifically the loci that were covered by a probe) was calculated from the Hi-C² matrix. Similarly, the average intrachromosomal coverage of all loci was calculated from the corrected genome-wide Hap1 wild-type Hi-C map. A matrix consisting of all entries corresponding to two loci that were both outside the target region was constructed from the raw uncorrected genome-wide Hap1 Hi-C map. This matrix was multiplied by the ratio of the average coverage of loci within the target region in the Hi-C² data to the average coverage of all loci from the genome-wide Hap1 wild-type Hi-C data and then summed with the Hi-C² matrix (thereby filling in the extremely sparse areas of the Hi-C² matrix). This summed matrix was then corrected with the KR matrix balancing algorithm. The resulting normalization factors may be used as correction factors for the Hi-C² data.
c. Raw gap-filling with rescaling: Filling in the sparse areas of the Hi-C² matrix corrects for first order target enrichment biases from hybrid capture to some extent, but does not account for the fact that differential enrichments may be present for entries of the matrix corresponding to one on-target loci and one off-target loci vs. entries corresponding to two on-target loci. To address this, the ratio of the number of contacts formed between the locus and off-target loci to the number of contacts formed between the locus and other on-target loci using the genome-wide Hap1 wild-type Hi-C data was first calculated before performing gap-filling as in the above methods. The same ratio was then calculated using the Hi-C² data. The ratio of these ratios provided a scaling factor for each on-target locus which was then used to scale all entries in the Hi-C² matrix corresponding to contacts between the on-target locus and off-target loci. After performing this correction, the method from above was followed, i.e., a matrix consisting of all entries corresponding to two loci that were both outside the target region was constructed from the raw uncorrected genome-wide Hap1 Hi-C map. This matrix was multiplied by the ratio of the average coverage of loci within the target region in the Hi-C² data (using the rescaled Hi-C² data) to the average coverage of all loci from the genome-wide Hap1 wild-type Hi-C data and then summed with the Hi-C² matrix (thereby filling in the extremely sparse areas of the Hi-C² matrix). This summed matrix was then corrected with the KR matrix balancing algorithm. The resulting normalization factors were used as correction factors for the Hi-C² data.
d. KR gap-filling with rescaling: This method is the same as method c, except that as in method b, the Hi-C² data was initially corrected with the KR factors derived from the Hap1 genome-wide wild-type Hi-C matrix and the KR corrected wild-type Hi-C data was used for gap-filling.
e. Raw gap-filling with rescaling and thresholding: It was noted that for a few very sparse (under-covered) rows in the Hi-C² data, the normalization methods would actually overcorrect, leading to highly-covered streak artifacts in the data. In order to remove these artifacts, a final filtering step was added where loci with a normalization factor (C) of less than 0.33 (where M_{i,j} is divided by Cᵢ and Cⱼ to get the corrected entry M*_{i,j}) were thresholded so that their normalization factors were raised to 0.33 (this was implemented after the KR matrix balancing was run, not as a constraint during the running of the algorithm). The threshold of 0.33 was chosen based on empirical observation of rows that led to streaky artifacts. This method is the same as method c except with the aforementioned thresholding.
f. KR gap-filling with rescaling and thresholding: This method is the same as method d except with the addition of the thresholding described in method e.

### Example 6—Mapping and Reconstruction of Chromothriptic Chromosomes

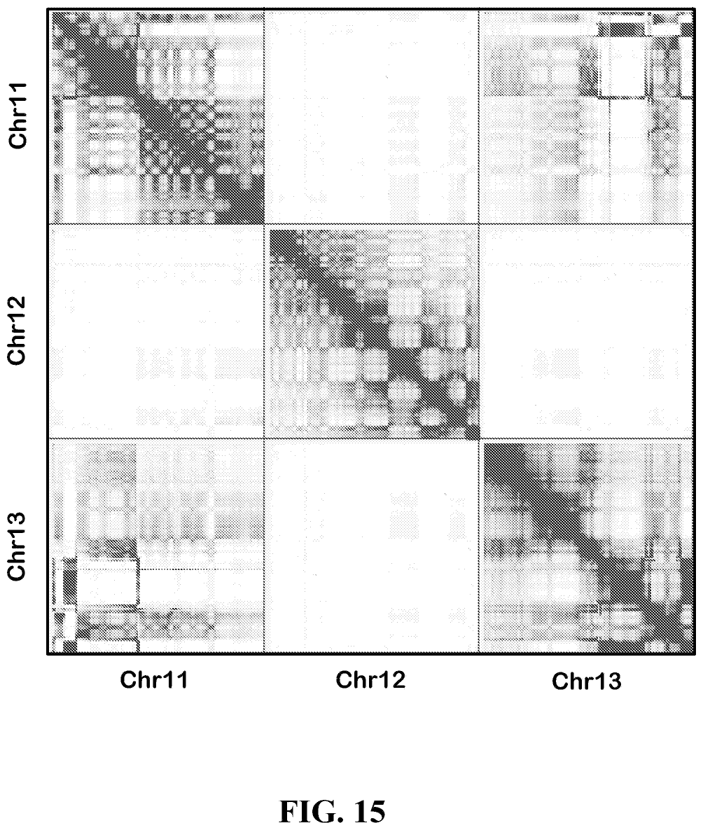

The methods (e.g., in situ HiC and intact Hi-C) described herein can measure the contact probability between pairs of genomic loci. Genomic segments that are physically connected tend to have a higher contact probability, inversely proportional to the linear distance between the loci. Genomic rearrangements on a map generated from a method described herein are easily discernible as sharp, asymmetrical peaks of contact probability. The direction of the asymmetrical peak can reveal the relative orientation of the rearranged chromosomal fragments. FIG. 15 shows an exemplary map generated using Hi-C of three mouse chromosomes including chromothripsis between chr11 and chr13. This is further illustrated in

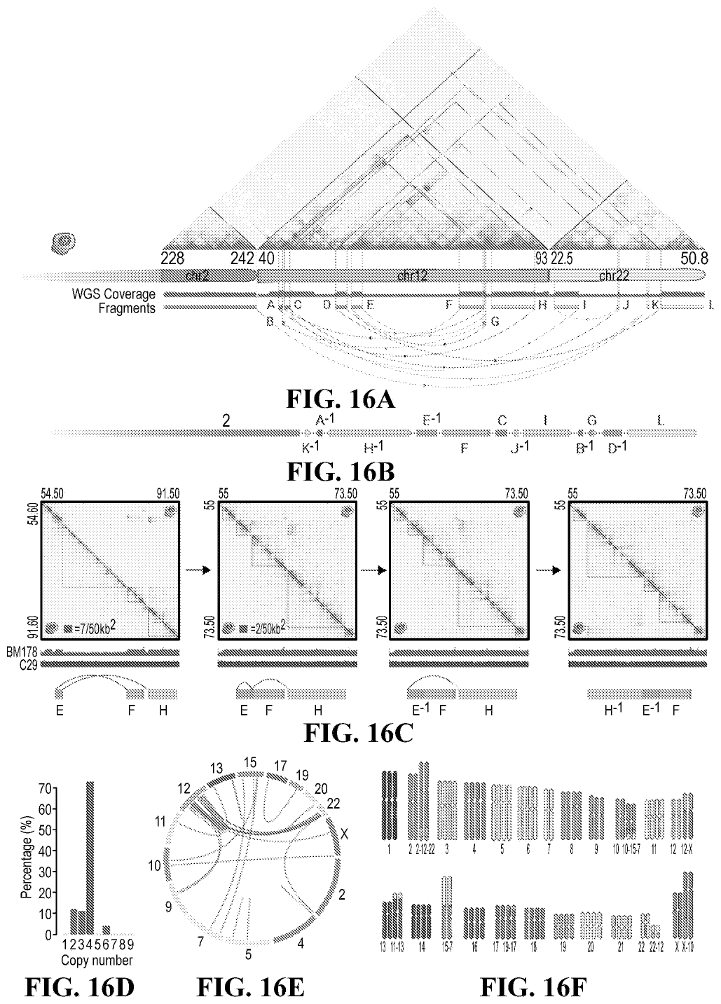

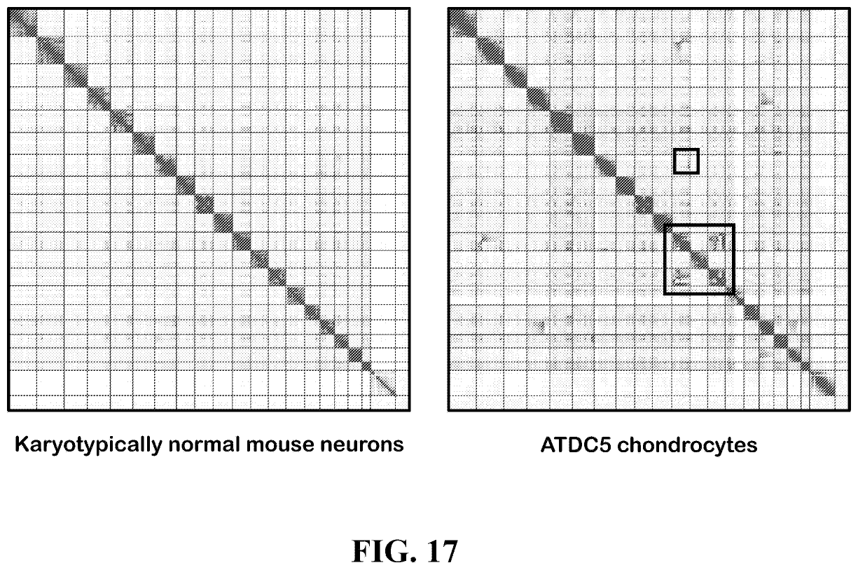

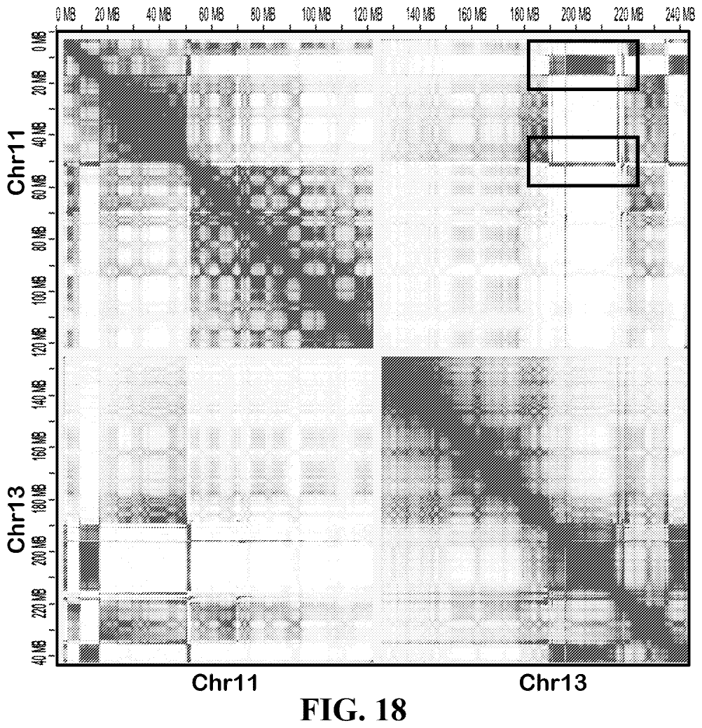

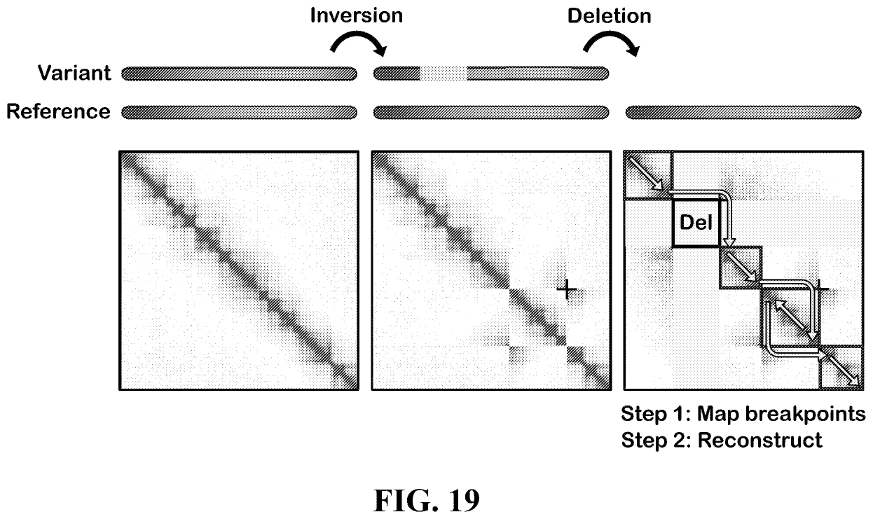

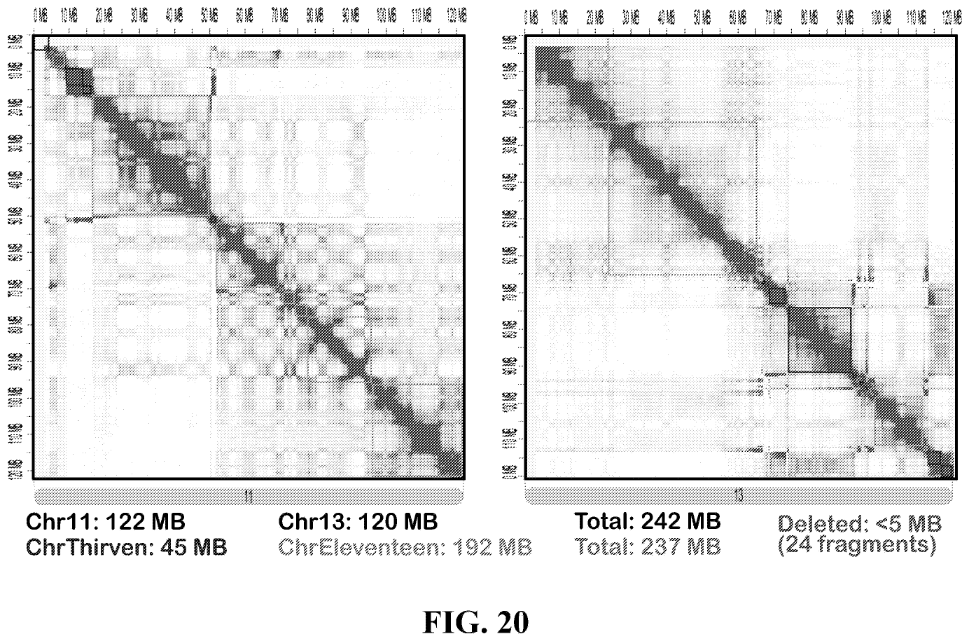

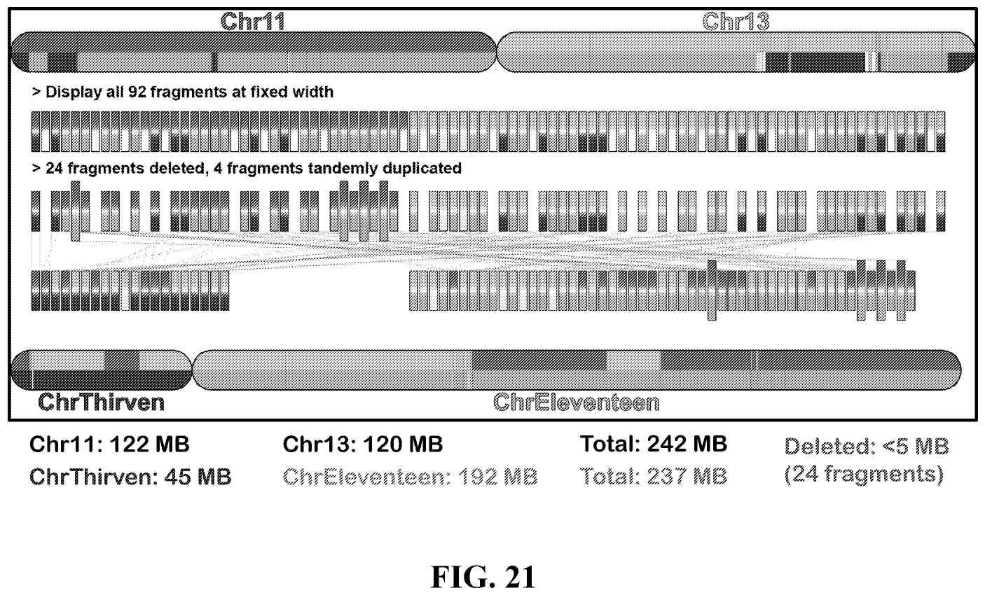

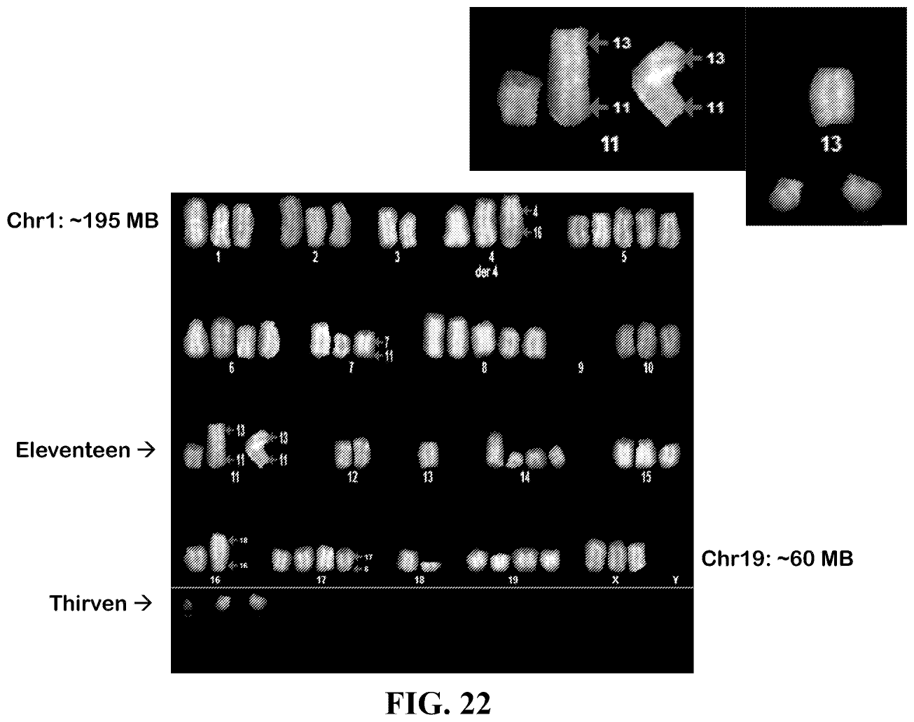

FIGS. 16A-16F can demonstrate that Hi-C can be used to ascertain the complete end-to-end structure of a chromothriptic chromosome using a relatively small amount of Hi-C data. The Hi-C map can reveal the positions of breakpoints in the genomic sequence, which separates the chromosome into unbroken, contiguous fragments. The Hi-C data further revealed the correct order and relative orientation of these fragments, as well as fragments that are deleted after the rearrangement. FIG. 17 can demonstrate that the genome-wide Hi-C map of ATDC5 chondrocytes shows unusual interchromosomal signals. FIG. 18 can demonstrate that ATDC5 chromosomes 11 and 13 (but not 12) show multiple rearrangements. FIG. 19 can demonstrate a procedure for reconstruction of complex genomic rearrangements. FIGS. 20-21 can demonstrate complete end-to-end reconstruction of chromosomes “thriven” (20 fragments) and “eleventeen” (55 fragments). FIG. 22 can demonstrate that chromosomes “thriven” and “eleventeen” appear in SKY data.

### Example 7—Phasing Genomes Using Hi-C and Other Methods Described Herein

Conventional phasing methods have certain limitations. Assisted methods are limited by the requirement for sequence trios and/or the reliance of population-based inferences, which require linkage information and are useful only in the normal state. De novo methods which have long reads make it difficult to recognize SNPs and pseudo-long reads do not produce chromosome-length haploblocks. Hi-C and other DNA proximity assays, such as any of those described in greater detail elsewhere herein can provide powerful sources of linking data.

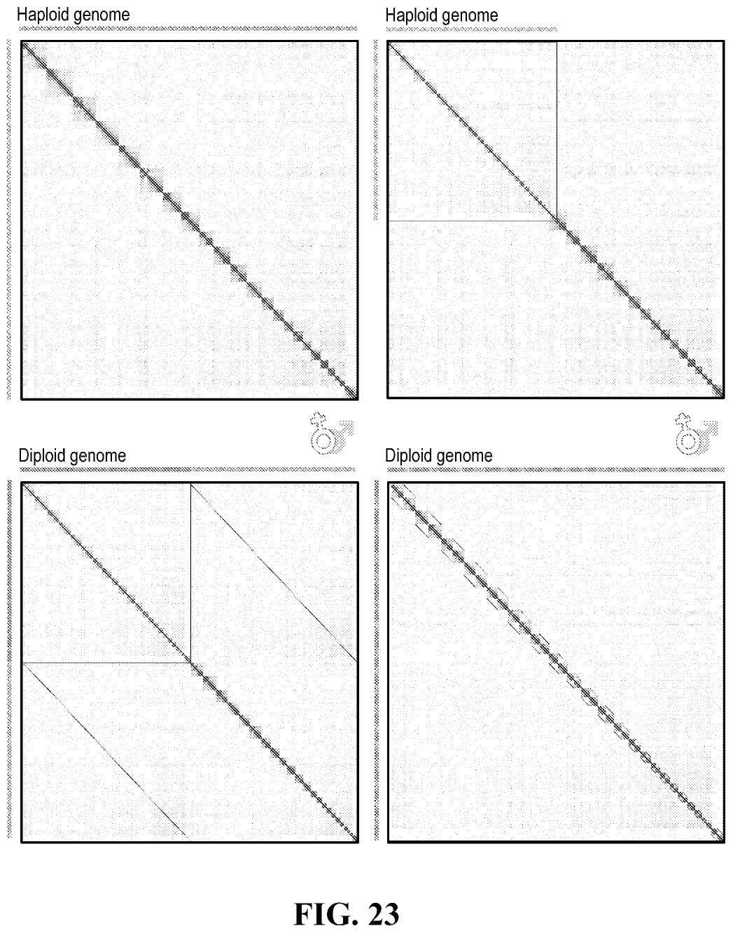

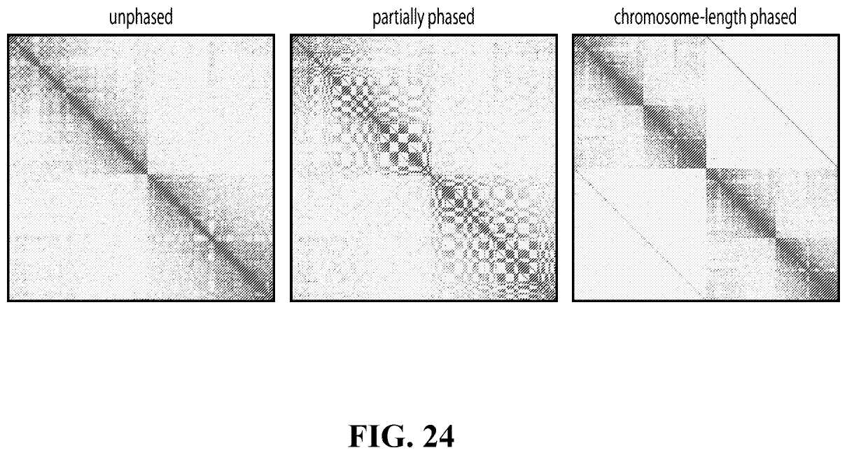

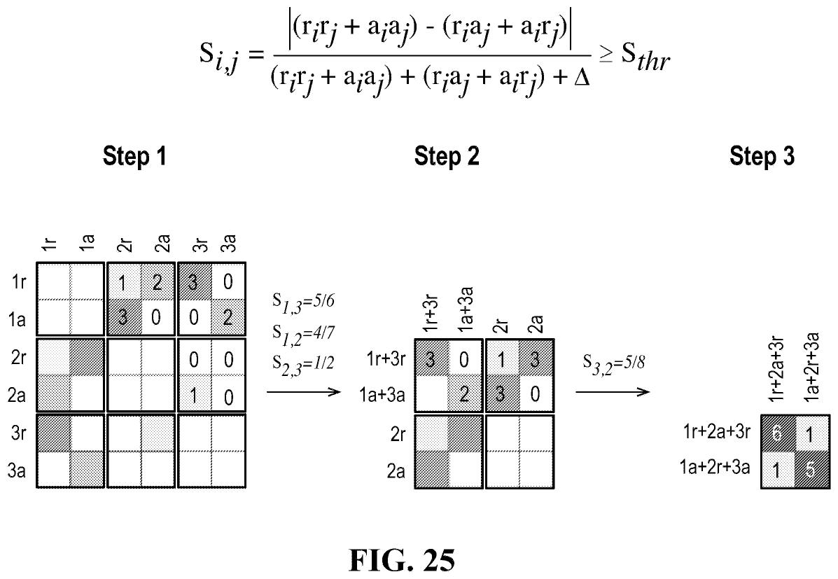

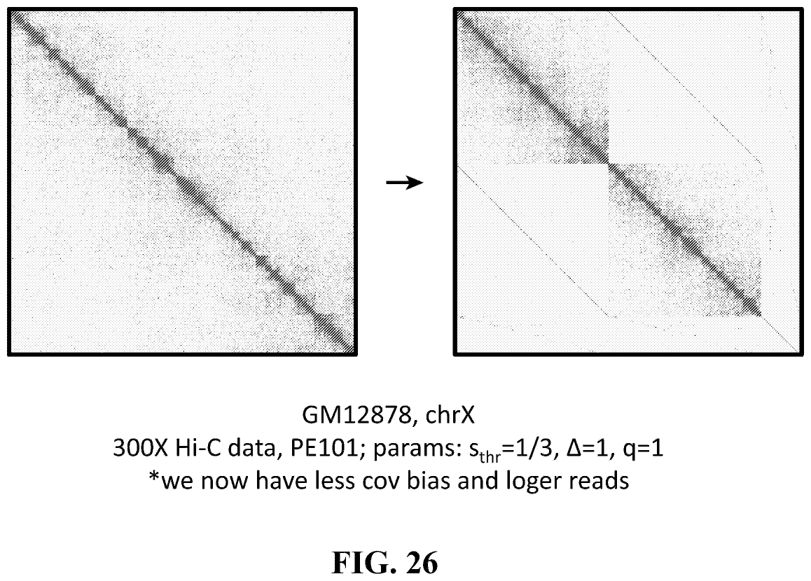

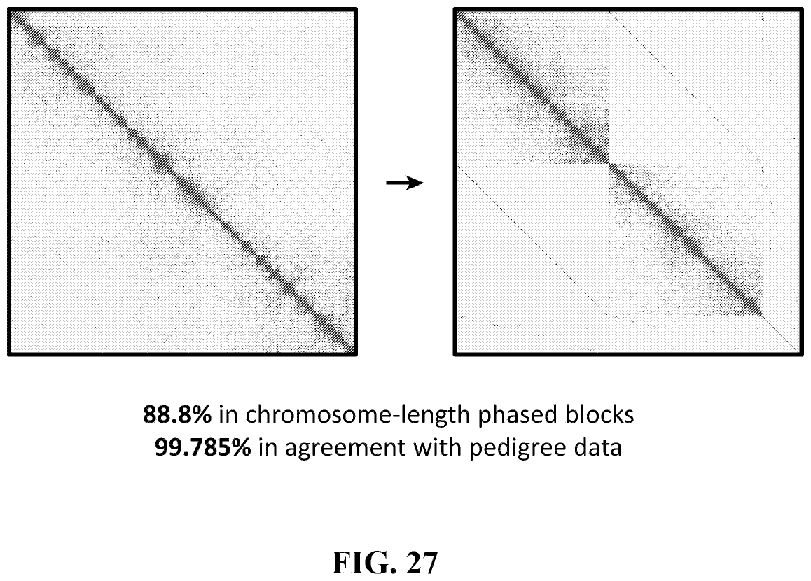

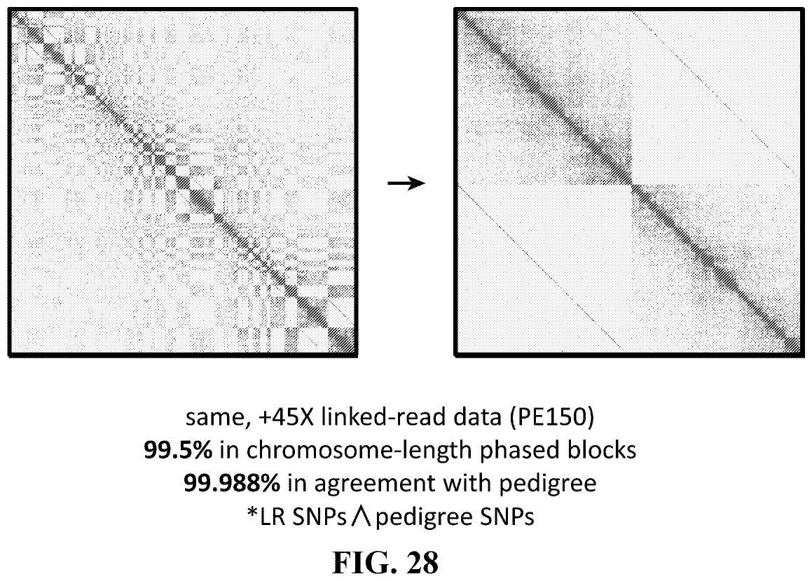

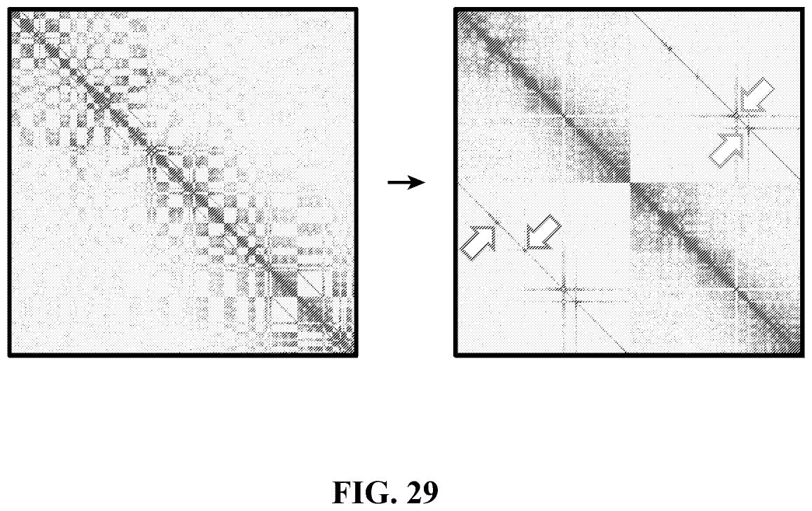

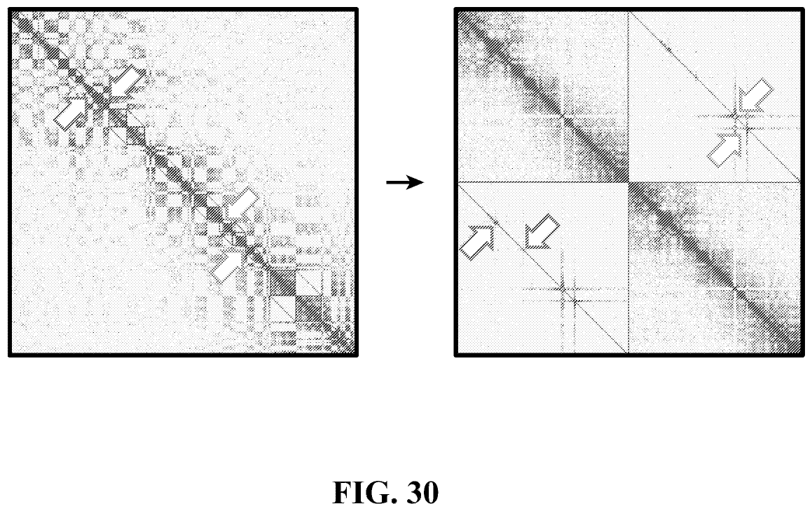

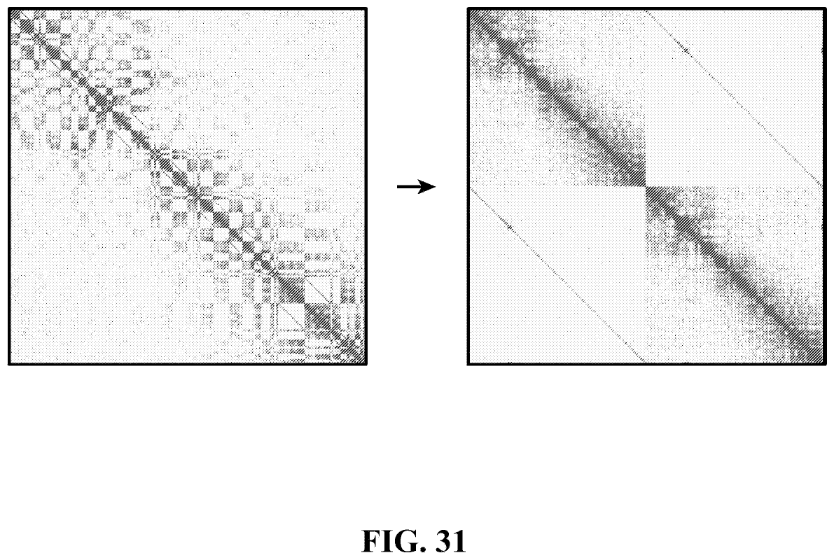

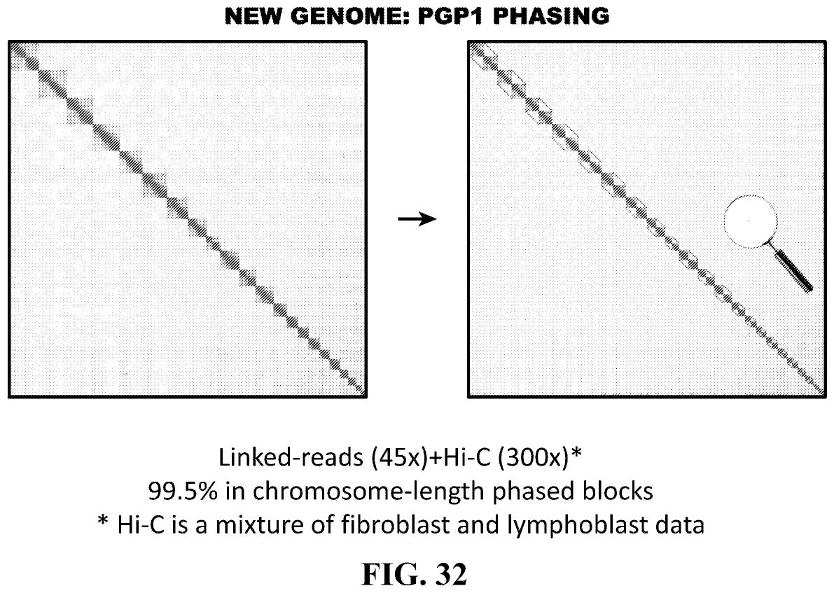

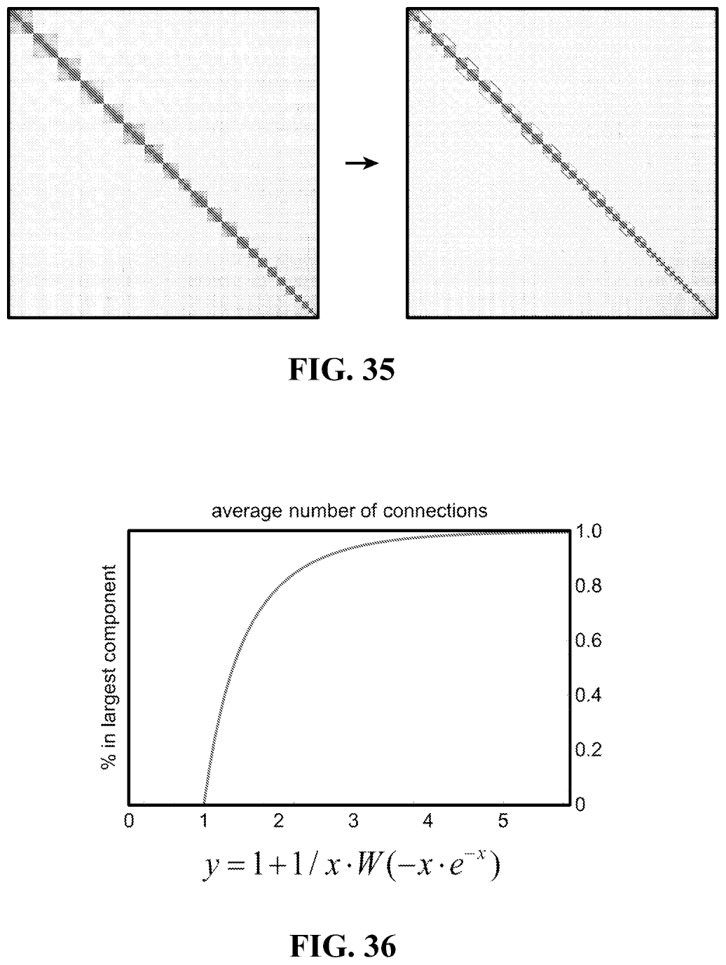

Data generated from the DNA proximity assays (e.g., Hi-C and others described herein) can be used to phase genome. As shown in FIG. 23, results from DNA proximity assays, such as Hi-C, are represented as a heatmap, like the map from a human sample shown in FIG. 23, where each entry shows how often two bits of a genome talk to each other on the scale from white to black, with black being more often. 23 squares are shown on the map of FIG. 23 reflecting the fact that loci on the same chromosome tend to talk to each other more often than to loci on other chromosomes. This is a helpful signal for assembly to anchor contigs to chromosomes. This Example can demonstrate a phasing module that can be applied to 3D genome data that utilizes a Hi-C signal or a signal from another DNA proximity method described in greater detail elsewhere herein. The phasing module can take as input: list of variants (.vcf) and/or list of dedupped alignments. This Example utilized dedupped Hi-C alignments (Juicer mnd file). Visual language can be used for evaluating phasing as shown in FIG. 24. The parameters were as follows: threshold enrichment score [default sₜₕ=⅓], relaxation parameter [default Δ=2], *threshold mapping quality [default qₜₕᵣ=1]. The module included Edge caller, Phaser (individual SNPs or pre-phased sets), a visualizer, and minimal necessary JBAT extension [not released]. FIG. 25 can show further attributes of the phaser module. As shown in FIGS. 26-27, the phaser generated chromosome-length phasing blocks with Hi-C data (FIG. 26) that agreed with pedigree data (FIG. 27). As shown in FIGS. 28-31, the phaser can take in other data types in addition to Hi-C(FIG. 28), generate chromosome and do error correction as needed, JBAT style (FIGS. 29-31). FIGS. 32-34 show phasing results from PGP1. FIG. 35 can demonstrate that the phaser can be used with 345× and 80× data. FIG. 36 shows a graph of the average number of connections v. % in largest component and may be helpful in determining how much and what data is needed for phasing using the methods described herein.

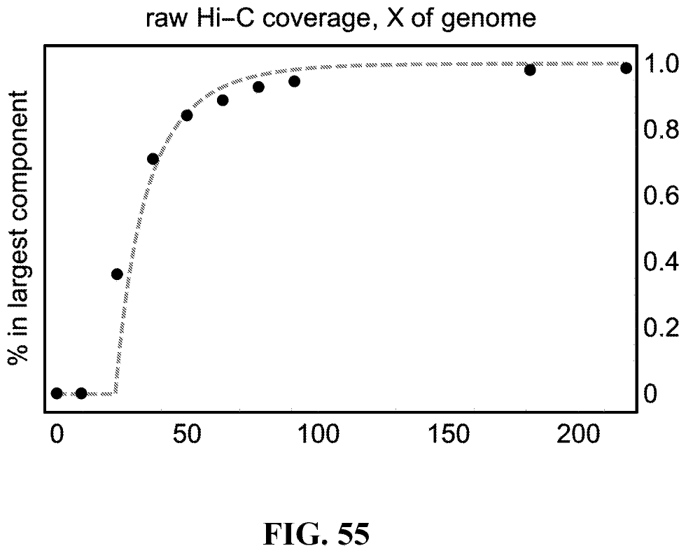

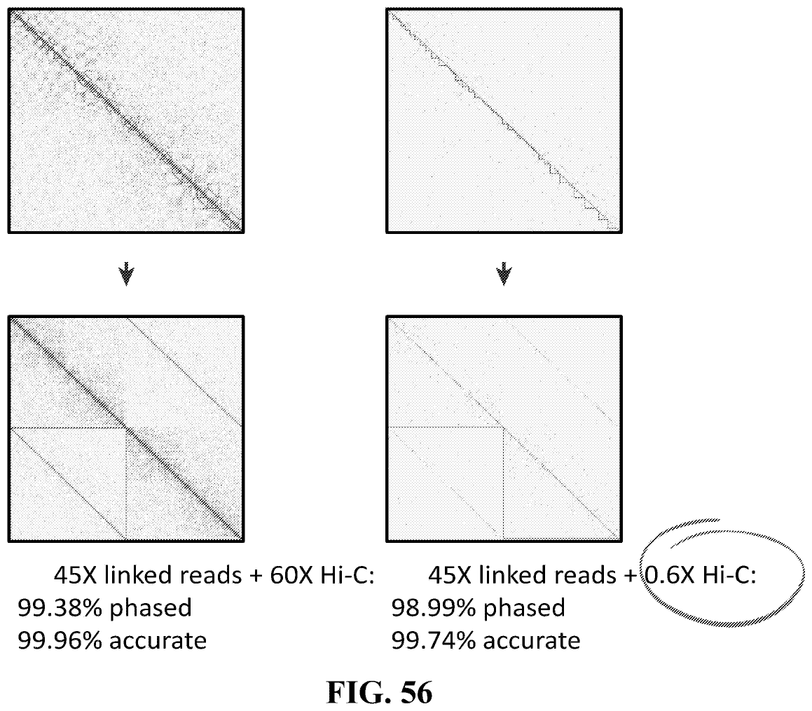

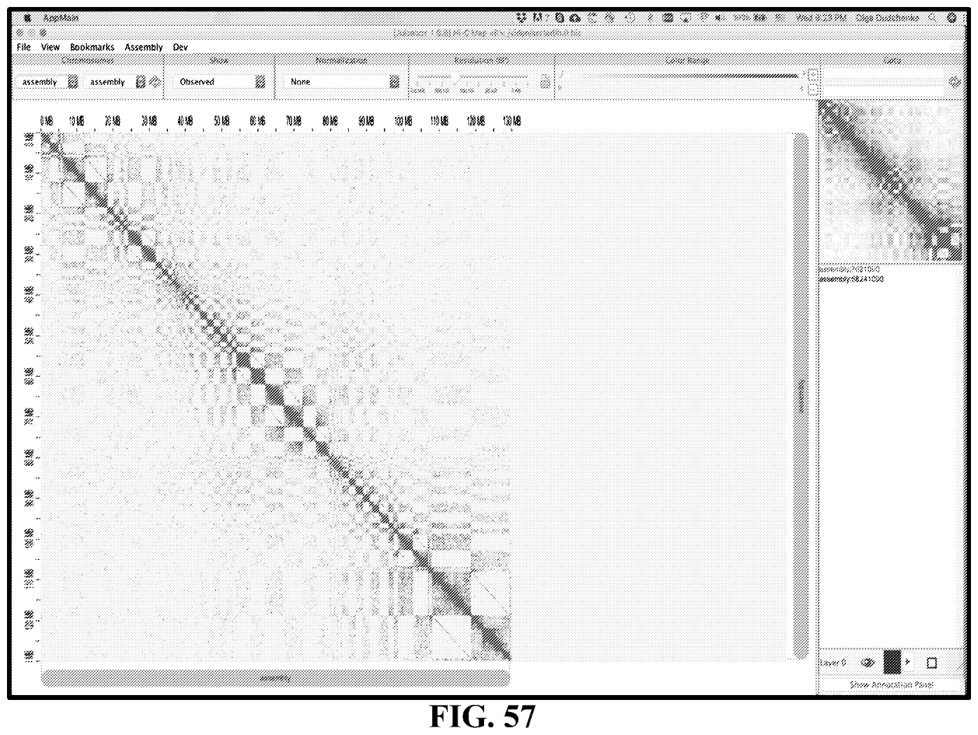

Applicants determined that 40×Hi-C reads are often enough to phase the majority of SNPs into chromosome-length haploblocks. (FIG. 55). Applicants determined that coverage requirement for phasing can be reduced to 0.6× for Hi-C (FIG. 56). Applicants developed an extension for the Juicebox software to allow for phasing (FIG. 57).

### Example 8—Personalized Genomes

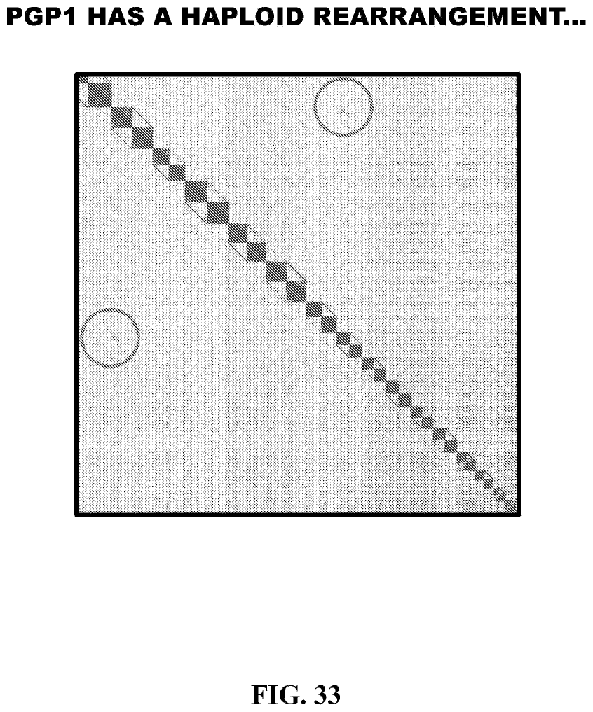

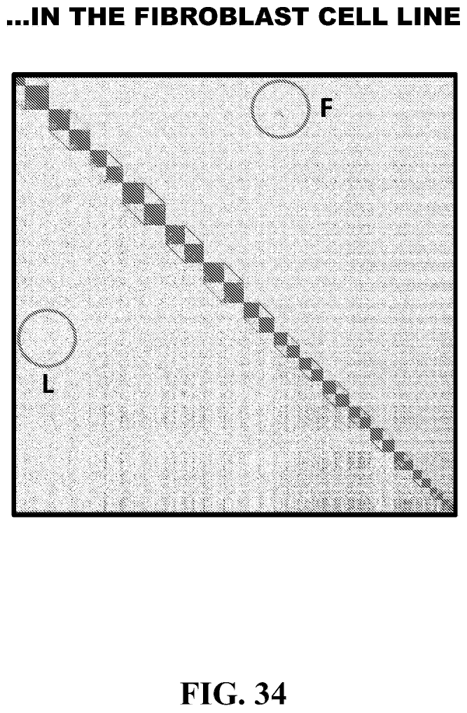

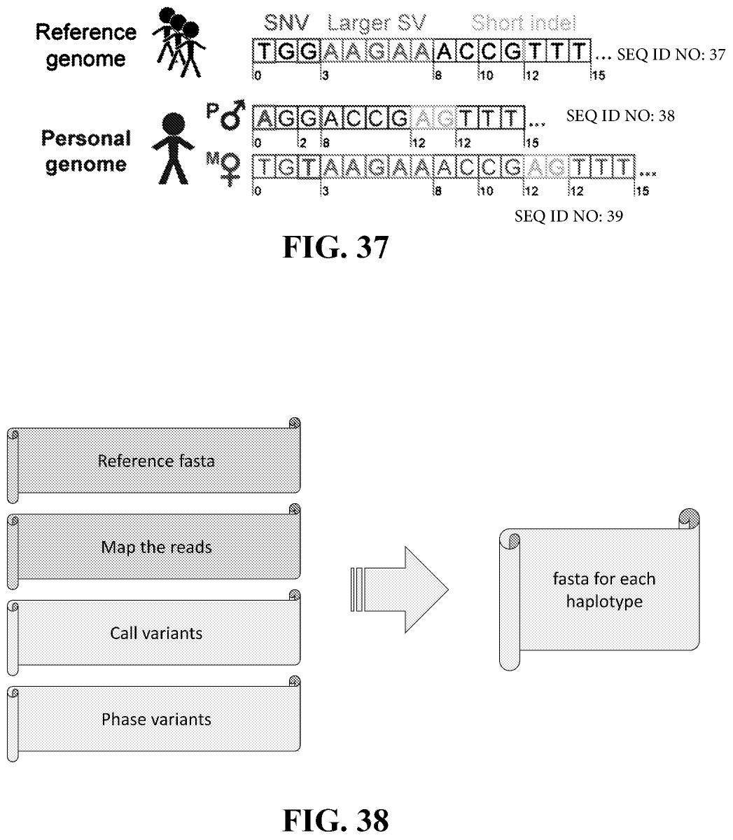

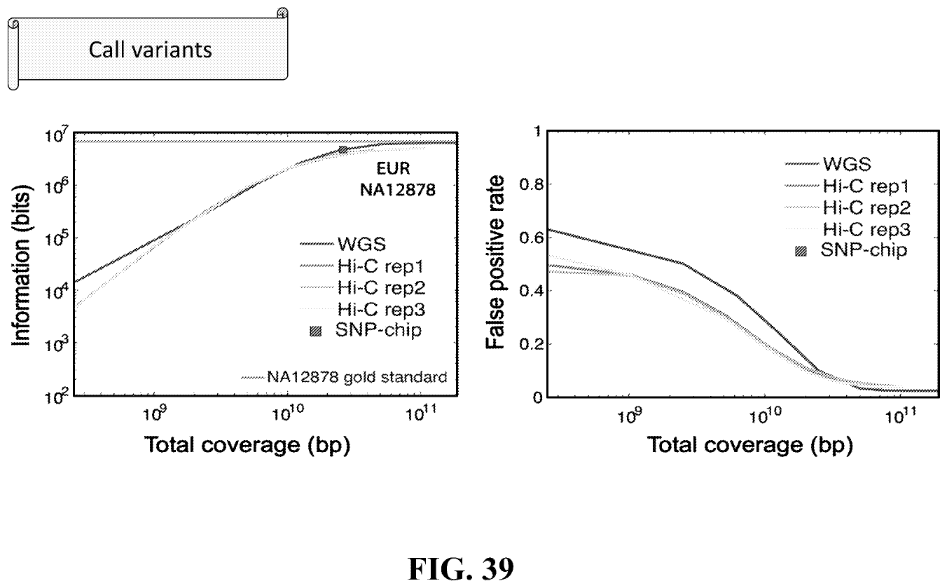

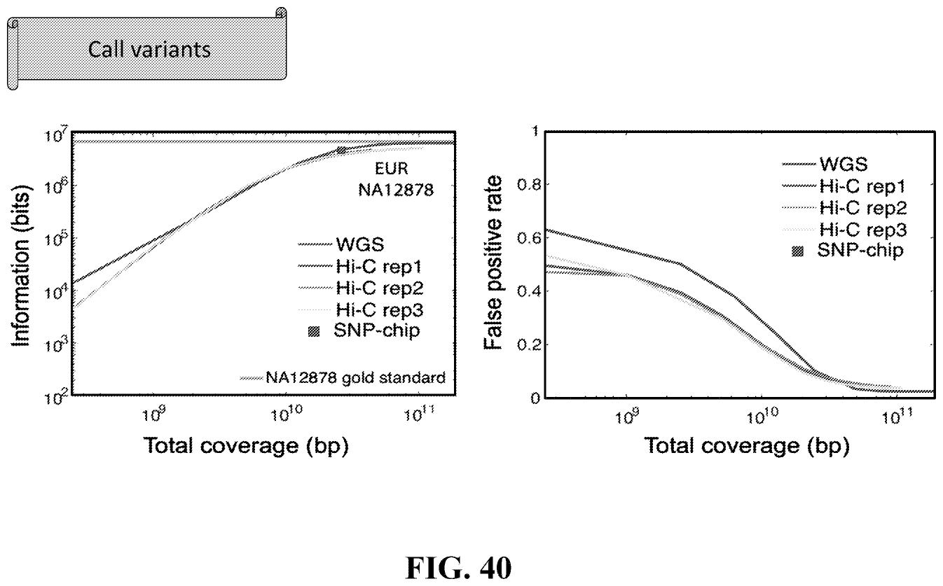

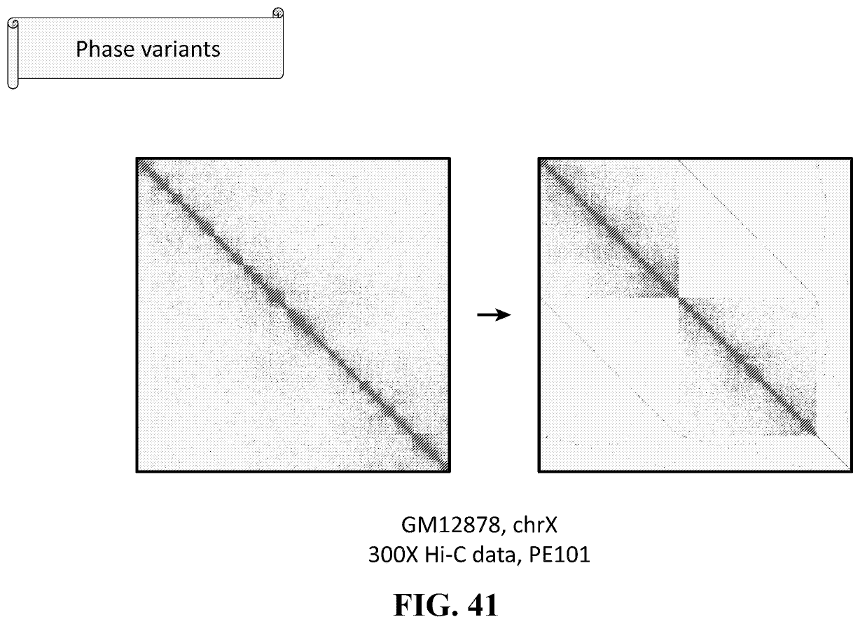

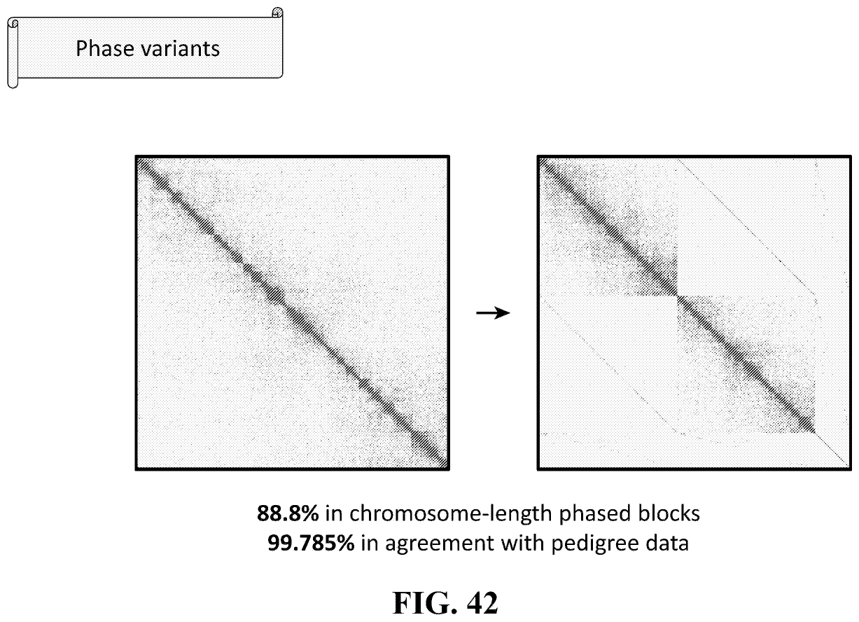

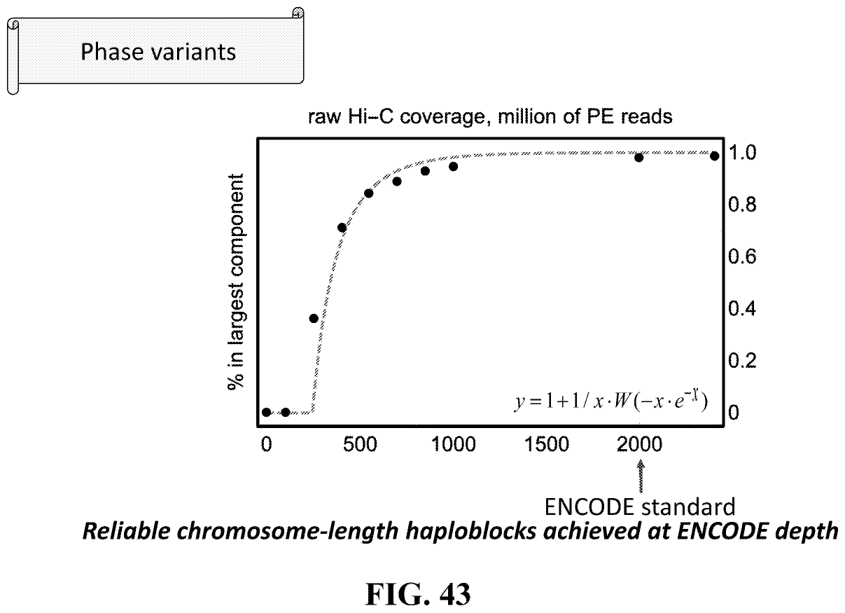

The DNA proximity assays and data analysis methods can be used therein for generation of a personalized genome (FIG. 37). FIGS. 33-34 show results from the production of diploid genomes with chromosome-length haploblocks and can demonstrate that PGP1 has a haploid rearrangement only in the fibroblast line. FIGS. 38-42 shows a flow diagrams representing a personalized genome pipeline (FIG. 38) and aspects thereof (FIGS. 39-42). FIG. 43 shows a graph that demonstrate the theoretical optimum of phaser approaches. The methods described herein also referred to as ENCODE Hi-C can utilize phased SNPs with chromosome length haploblocks for generation of a personalized genome.

Applicants can use Hi-C to generate a genome for any organism de novo. Applicants used Hi-C to assemble de novo an $1K diploid genome of Stenella frontalis (dolphin @DNA Zoo) (FIG. 58).

### Example 9—Deep Learning for Genome Wide Analysis of Hi-C Maps

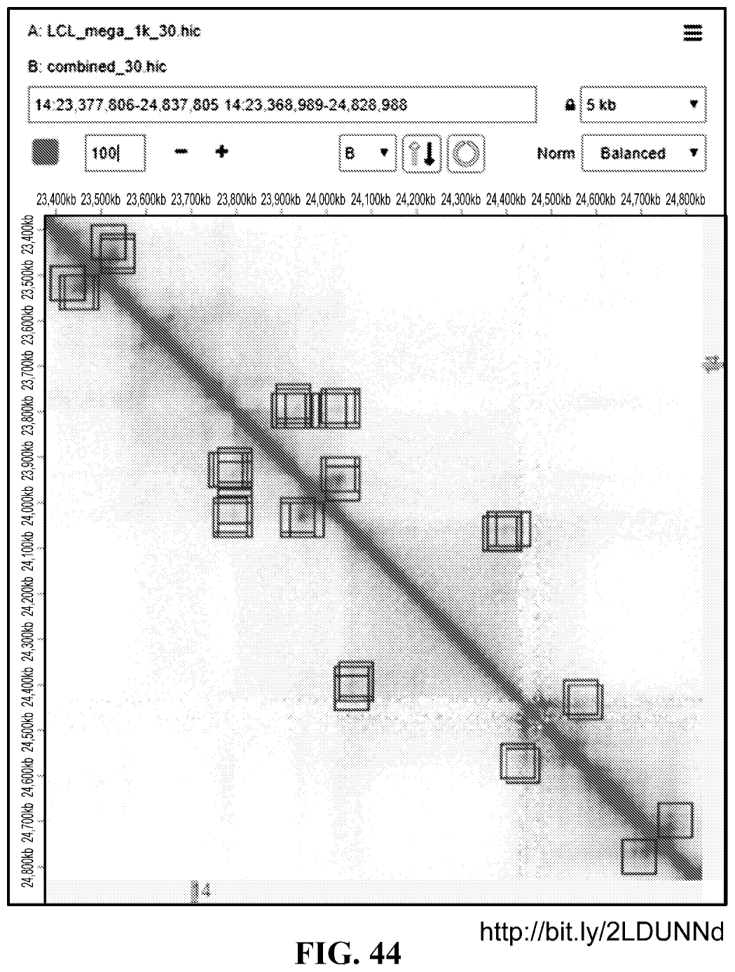

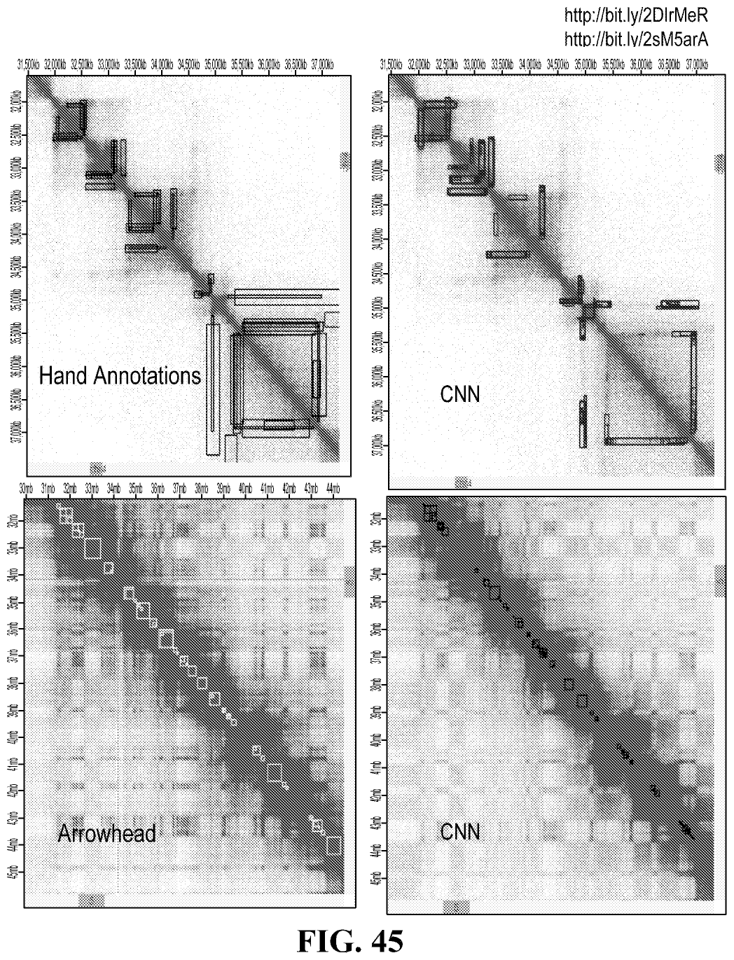

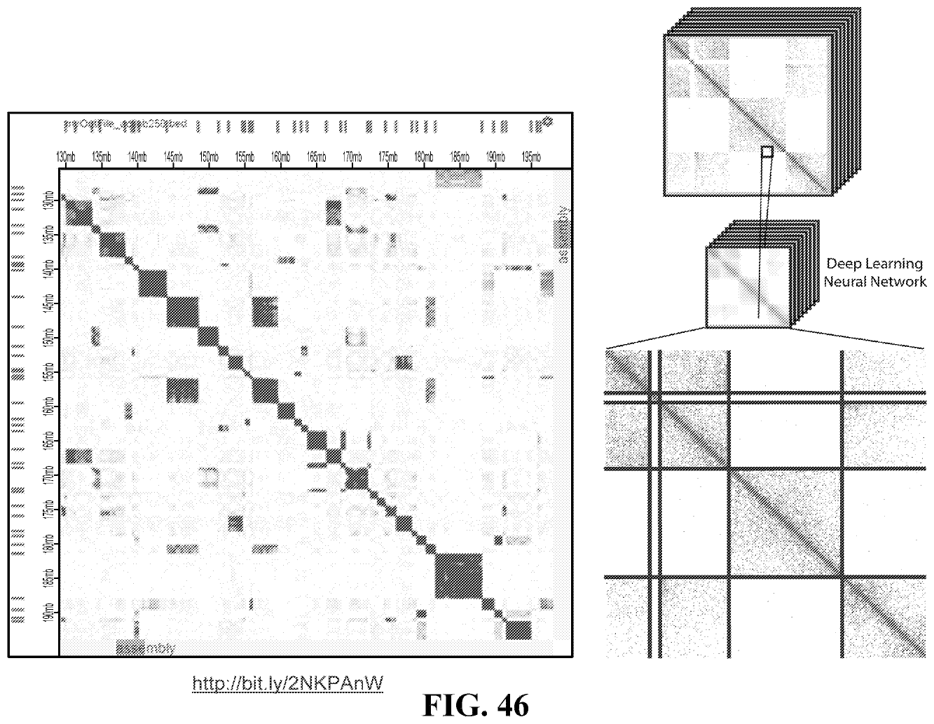

FIGS. 44-46 can demonstrate the use of deep learning for genome wide analysis of Hi-C and other DNA proximity assay maps. As shown in FIG. 44, a CNN was used to detect small local chromatin structures like loops. As shown in FIG. 45 a CNN can also be used to detect larger chromatin features like stripes and contact domains. As shown in FIG. 46, Hi-C data and deep learning can be used to predict genetic 1D tracks. This can be useful for an enhanced 3D-DNA pipeline with deep-learning-based misjoin correction and/or Chip-SEQ prediction from chromatin structural data.

### Example 10— Hi-C Performed in Less than 24 Hours

The following is a modified protocol allowing the Hi-C protocol to be performed in less than 24 hours. The Hi-C reaction was reduced to a single tube reaction for restriction, biotin incorporation, and ligation, to produce a Hi-C library for next-generation sequencing. Originally these steps took more than four hours to complete. Further, in this modified protocol the crosslink reversal step is no longer performed without a decrease in the quality of the genome assembly obtained. Further modifications have been made to the sequencing library preparation step. Specifically, end repair, A-tailing, and sequencing adapter ligation are now done in a single reaction. The further cuts down on overall time and expense. The ability to complete these steps in a single reaction was also unexpected given expectations in the art that DNA can detach from beads because of the higher temperatures needed for the single reaction chemistries and that magnetic beads (both Ti beads and SPRI beads) can inhibit certain reactions. For example, it is known that some T1 beads and SPR1 beads can affect polymerase chain reaction leading to library failure.

### Permeabilization

Prepare a stock solution of 1×Hi-C lysis buffer, and cool on ice:

Hi-C Lysis Buffer
38.72 ml of water
400 μl of 1M Tris-HCl pH 8.0 [final: 10 mM]
80 μl of 5M NaCl [final: 10 mM]
800 μl of 10% Igepal CA630 [final: 0.2%]

Gently suspend sample in 500 μl of Hi-C lysis buffer.

Incubate samples in 4° C. cold room with slow rotation for 5 minutes.

Centrifuge at 3000×G, 25° C. for 5 minutes.

To permeabilize the nuclear membrane and solubilize proteins, resuspend pellet in 50 μl of 0.5% sodium dodecyl sulfate (SDS) and incubate at 62° C. for 10 minutes.

Add 150 μl of water, 25 μl of 10% Triton X-100, and 25 μl of 10×T4 DNA Ligase buffer to quench SDS. Mix well by pipetting, avoiding excessive foaming. Incubate at 37° C. for 10 minutes.

Add 25 μl of 10× NEBuffer 2 (New England BioLabs [NEB], B7002S).

Centrifuge at 3000×G, 25° C. for 5 minutes.

### Hi-C Reaction

Discard the supernatant from step 8 and resuspend the pellet into 75 μl of following Hi-C Master Mix and incubate at 37° C. for 50 minutes without mixing:

30 μl of water

7.5 μl of 10×T4 DNA Ligase buffer (or NEB Cutsmart)

3.5 μl of 10% Triton X-100

8 μl of 1 mM biotin-11-dUTP

8 μl of 1 mM dATP

4 μl 1 mM dCTP

4 μl 1 mM dGTP

2 μl of 5 U/μl DNA Polymerase I, Large (Klenow) Fragment (NEB, M0210)

1 μl MseI (R0525M, 50 U/μ1)

2 μl MboI (NEB, R0147)

5 μl T4 DNA ligase (NEB, M0202L)

Add 2 μl 0.5M EDTA and mix well to stop the reactions before centrifugation.

Spin for 3000×G, 25° C. for 5 minutes. Pipet out and discard supernatant.

### DNA Extraction

Resuspend nuclear pellet in 180 μl Qiagen ATL Buffer and transfer to a Qiagen Pathogen Lysis Tube (S) (Qiagen, 19091).

Bead beat sample for 5 minutes.

Add 20 μl proteinase K, mix by vertexing and incubate at 56° C. for 30 minutes.

Add 200 μl Buffer A L. Mix thoroughly by vortexing. Incubate blood samples at 56° C. for 10 min

Add 200 μl ethanol (96-100%). Mix thoroughly by vortexing.

Pipet the mixture into a DNeasy Mini spin column placed in a 2 ml collection tube. Centrifuge at ≥6000×g (8000 rpm) for 1 min. Discard the flow-through and collection tube.

Place the spin column in a new 2 ml collection tube. Add 500 μl Buffer AW1. Centrifuge for 1 min at ≥6000×g. Discard the flow-through and collection tube.

Place the spin column in a new 2 ml collection tube, add 500 μl Buffer AW2 and centrifuge for 3 min at 20,000×g (14,000 rpm). Discard the flow-through and collection tube.

Transfer the spin column to a new 1.5 ml or 2 ml microcentrifuge tube.

Elute the DNA by adding 130 μl Buffer AE to the center of the spin column membrane.

Incubate for 1 min at room temperature (15-25° C.). Centrifuge for 1 min at ≥6000×g.

### DNA Shearing

Transfer the entire 130 μlto a Covaris microTUBE (tube capacity 130 ul). To make the biotinylated DNA suitable for high-throughput sequencing using Illumina sequencers, shear to size of 300-500 bp using Covaris instrument.

Instrument: M220 Focused-ultrasonicator (Covaris)

Peak Power: 70.0

Duty Factor: 20.0

Cycles/Burst: 500

Duration: 110 seconds

After shearing, remove the cap of the Covaris tube and transfer solution to a 1.5 ml tube.

### Hi-C Library Enrichment

Prepare in advance the following buffer:

2× Tween Binding and Washing Buffer (2×TWB):
10 mM Tris-HCl (pH 8)
1 mM EDTA; 2M NaCl
0.1% Tween 20

Take 50 μl of 10 mg/ml Dynabeads MyOne Streptavidin T1 beads (Thermo Fisher, 65602) per Hi-C library and separate on a magnet discarding the storage solution.

Resuspend the beads in 2×TWB, reclaim beads, and discard wash buffer. Perform all the following steps in low-bind tubes.

Resuspend the beads in 130 μl of 2×TWB and add to the tube with 130 μl sheared library from step 25. Incubate at room temperature for 15 minutes mixing at 650 RPM to bind biotinylated DNA to the streptavidin beads.

Separate on a magnet and discard the solution.

Resuspend beads in 500 μl Tris-Tween buffer. Separate on a magnet and discard the solution.

Resuspend beads in 25 μl Tris-Tween buffer.

### Sequence Library Preparation

Add 25 μl of ClaSeek End Conversion Master Mix to sample from step 32.

Incubate the mixture in a thermal cycler (lid temperature 100° C.) for 5 minutes at 20° C., followed by 10 minutes incubation at 72° C., and hold reaction at 4° C.

Supplement the reaction mixture from step 34 with 5 μl of water, 5 μl Illumina compatible adapters, and 10 μl ClaSeek Ligation Mix. Keep the mixture on ice. Mix the contents by vortexing (3-5 seconds) and spin down to the bottom of the tube.

Incubate the mixture at room temperature for 5 minutes

Separate on a magnet and discard the solution.

Resuspend beads in 200 μl Tris-Tween buffer. Separate on a magnet and discard the solution.

Resuspend beads in 200 μl Tris-Tween buffer. Separate on a magnet and discard the solution.

### Polymerase Chain Reaction and Final Library Clean UP

Resuspend beads from step 39 in 100 μl of PCR master mix:

50 μl of 2× Kapa HiFi HotStart ReadyMix (KAPA Biosystems, KK2602)

40 μl of water

10 μl of 25 μM Illumina primer mix

To amplify the Hi-C library run the following PCR protocol:
98° C. for 45 sec
cycle 6-12 times (10 cycles):
   98° C. for 15 sec
   55° C. for 30 sec
   72° C. for 30 sec
72° C. for 1 min
4° C. indefinitely

Warm a bottle of SPRI beads to room temperature. Shake to resuspend fully the magnetic beads.

Pool both PCR aliquots in one tube. Add 100 μl of room temperature SPRI beads (1× volume). Mix well brief vortexing and touch spin the tubes, incubate at room temperature for 10 minutes, separate on a magnet, and remove the supernatant.

Keeping the beads on the magnet, wash twice for 30 seconds with 500 μl of freshly prepared 70% ethanol without mixing the beads. Remove the ethanol completely.

Resuspend the beads in 100 μl of 1× Tris buffer and incubate to solubilize DNA. To the same tube, add 100 μl of 20% PEG 8000, 2.5M NaCl room temperature solution equivalent to 1×SPRI. Mix well by pipetting, incubate at room temperature for 10 minutes, separate on a magnet, and remove the supernatant.

Keeping the beads on the magnet, wash twice for 30 seconds with 200 μl of freshly prepared 70% ethanol without mixing. Remove the ethanol completely.

Resuspend the beads in 30-50 μl of 1× Tris buffer to elute DNA. Incubate at room temperature for 2 minutes, separate on a magnet, and transfer the supernatant to a fresh labeled tube.

Measure the DNA concentration of each final Hi-C library. Use Agilent Bioanalyzer to estimate the average fragment size the final molarity of each library. For accurate quantification before sequencing, use a qPCR Illumina Library Quantification Kit (KAPA Biosystems, KK4824).

The Hi-C final libraries are now ready for Illumina paired end sequencing.

### Example 11—Hi-C is a WGS Assay that can be Used to Call SNPs

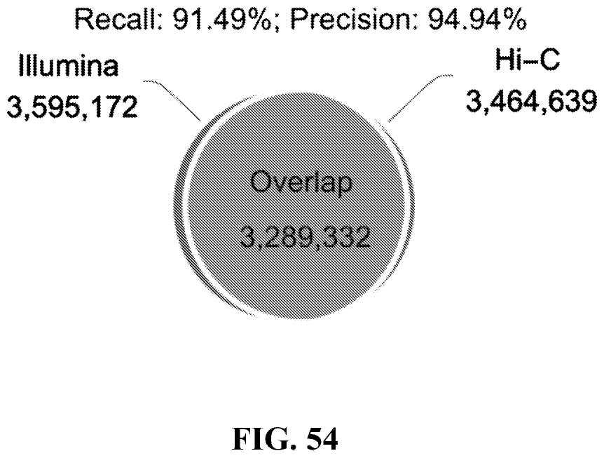

Applicants compared SNP calls from 25×MNase Hi-C data with the Illumina Platinum Genomes SNP calls validated using haplotype inheritance information through a well-studied 17-member pedigree (CEPH 1463) (FIG. 54). The reads between the two methods overlapped. Thus, Hi-C can be used for whole genome sequencing, but also providing additional information and uses, such as phasing, 3D architecture, and genome assembly. Applicants determined the number of Hi-C reads required to have 100% coverage of the genome (FIG. 55).

### Example 12—Intact Hi-C

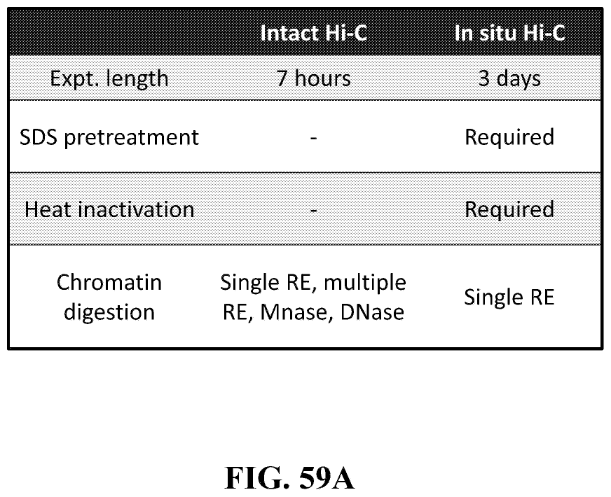

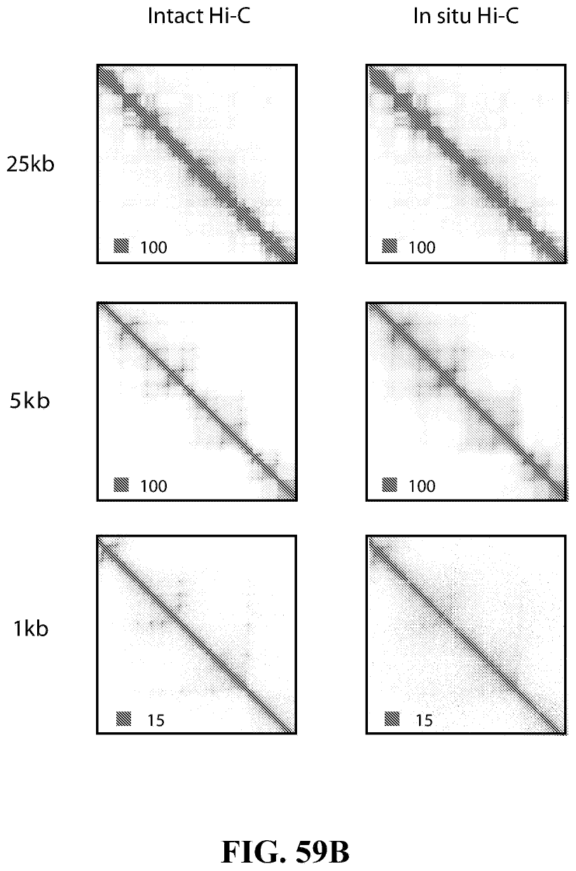

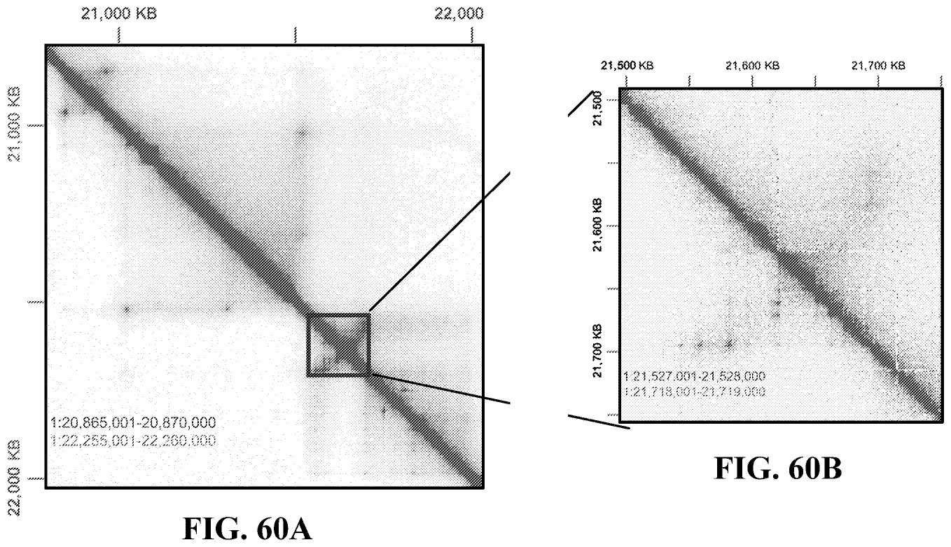

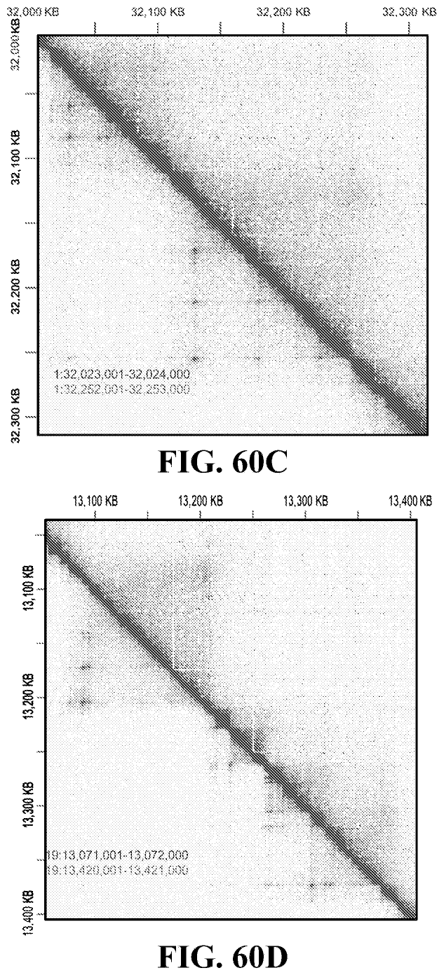

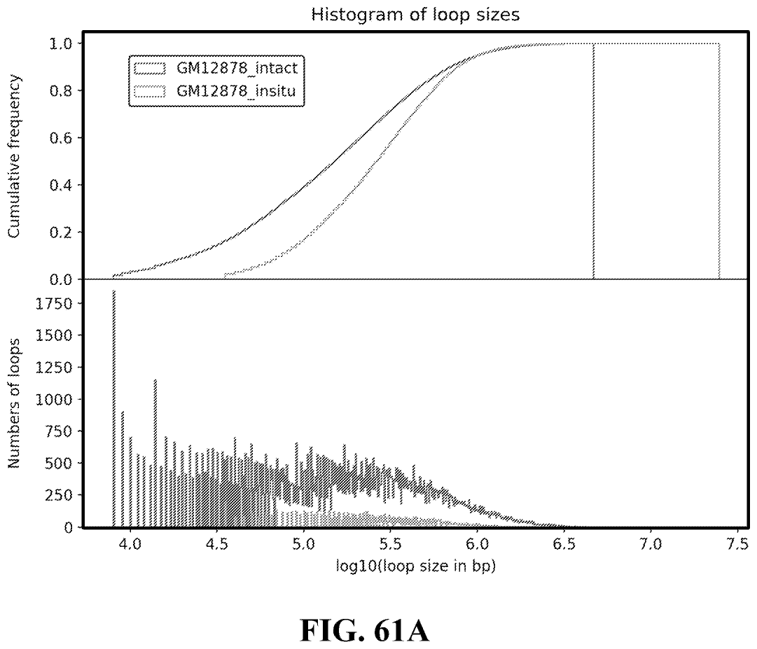

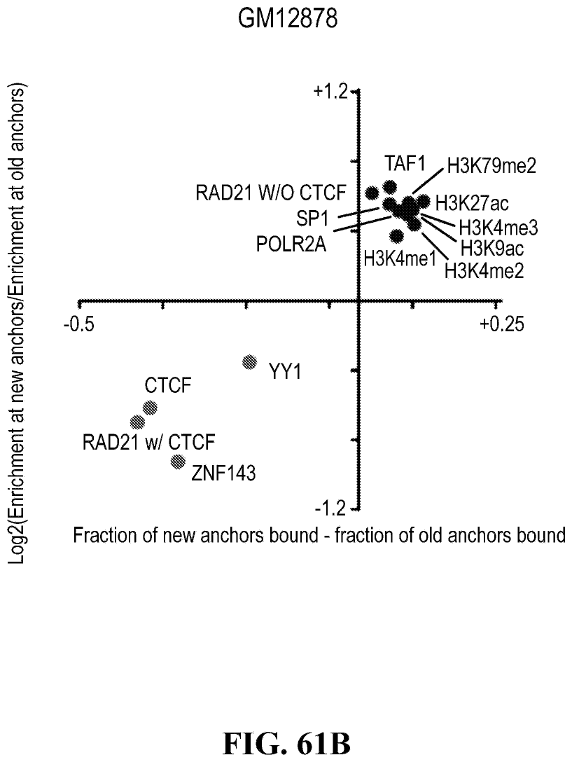

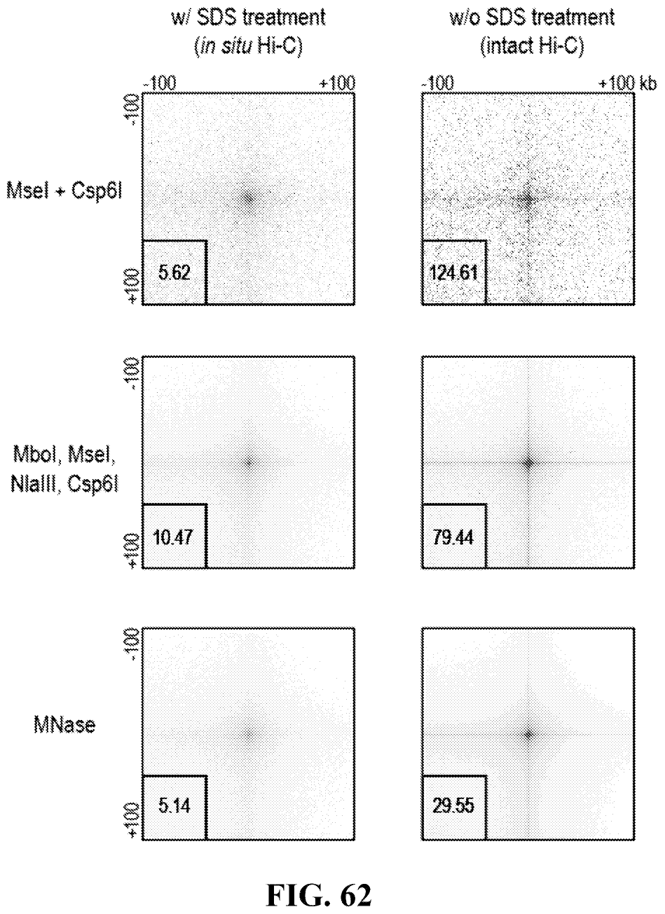

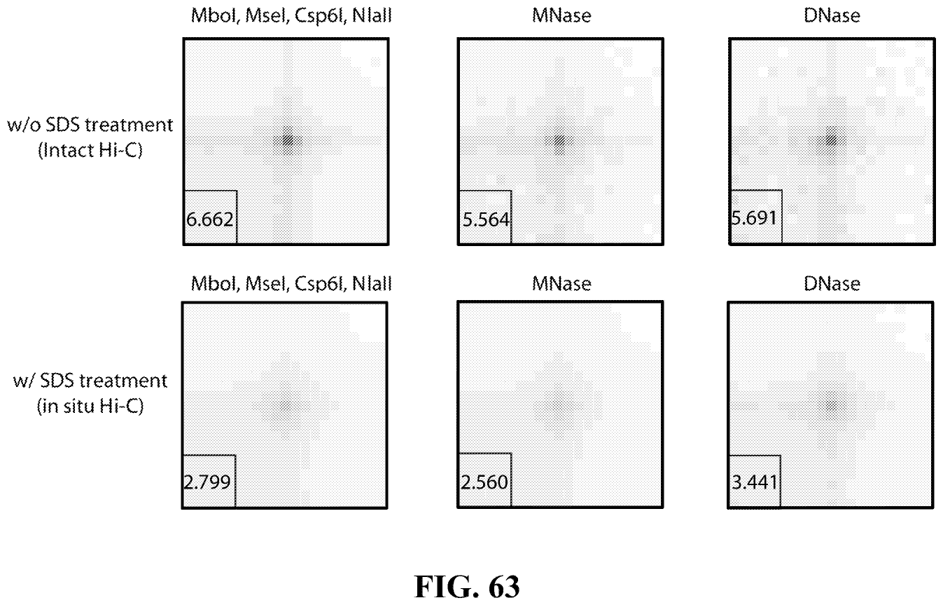

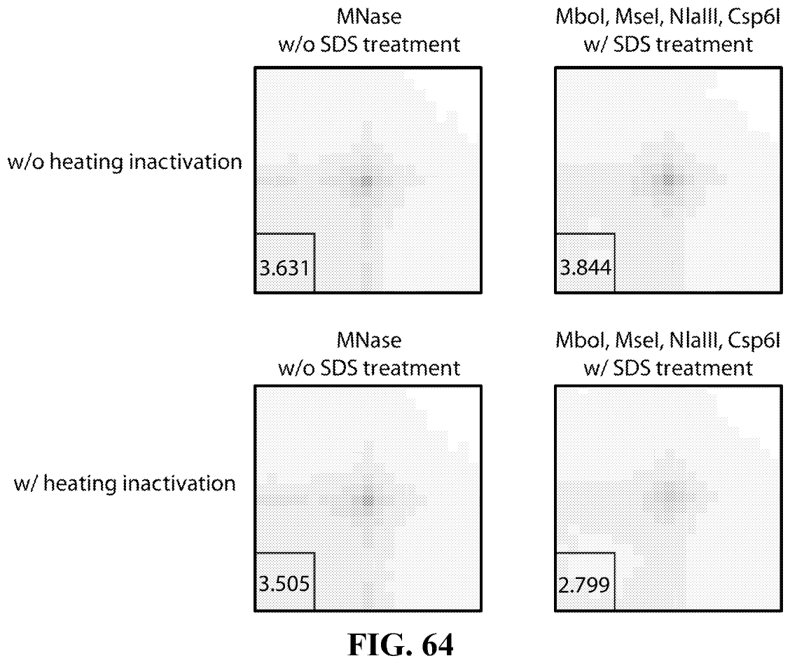

Applicants developed a new method for identifying contacts within a genome. The method does not require SDS or heat inactivation for permeabilization of nuclei (FIG. 59A) and detects the same contacts as in situ Hi-C (FIG. 59B). Intact Hi-C identifies many more loops as compared to in situ Hi-C (FIG. 60A-D, 61A). Moreover, the loops that were identified with in situ Hi-C are enriched for CTCF, whereas the newly identified loops are enriched for transcription factors and histone modifications associated with transcription activation (FIG. 61B). Thus, CTCF independent loops are identified and non-CTCF proteins capable of arresting cohesin at the loops are also identified. For example, RNA polymerase (POLR2A), the SP1 activator protein, and TFIID (TAF1) are identified at loop anchors. Furthermore, histone H3 lysine methylation was identified at loop anchors. Histone H3 lysine methylation is associated with active transcription (H3K4me3) and can recruit methyl-binding proteins to the loop anchor (see, e.g., Zhang T, Cooper S, Brockdorff N. The interplay of histone modifications—writers that read. EMBO Rep. 2015; 16(11):1467-1481). This data shows that loop anchors are present at actively transcribed genes and provides further evidence for promoter enhancer loops. SDS is commonly used to permeabilize nuclei and it was considered routine to permeabilize nuclei with SDS and heat for in situ Hi-C. Applicants determined for the first time that SDS in a Hi-C assay caused DNA fragments in contact or in spatial proximity to fall apart, possibly due to loss of cohesin complex integrity (FIGS. 62 and 63). Moreover, heating in the presence of SDS also reduces the loop signal (FIG. 64). Applicants identified conditions for permeabilizing the nuclei for performing the in situ steps (fragmenting, fill in/end repair and ligation) that maintains the contacts in the nuclei.

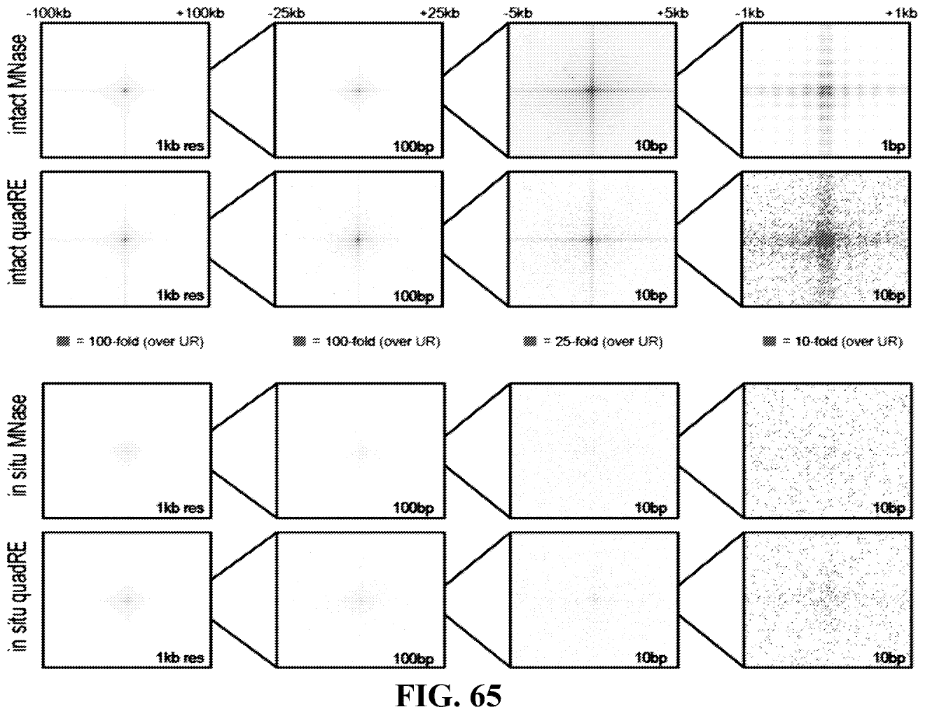

Intact Hi-C allows for determining base pair resolution, such that it allows for determining that two single base pairs are in contact (FIG. 65). In analyzing many contacts 10 base pair resolution is capable of determining that nucleotides in a ten base pair sequence are in contact with nucleotides in another 10 base pair sequence. CTCF mediated peaks localized to their exact motifs in 2014 show punctate contact enrichment directly over the motif in intact Hi-C, whereas the enrichment is much weaker and more diffuse in in situ Hi-C (FIG. 65).

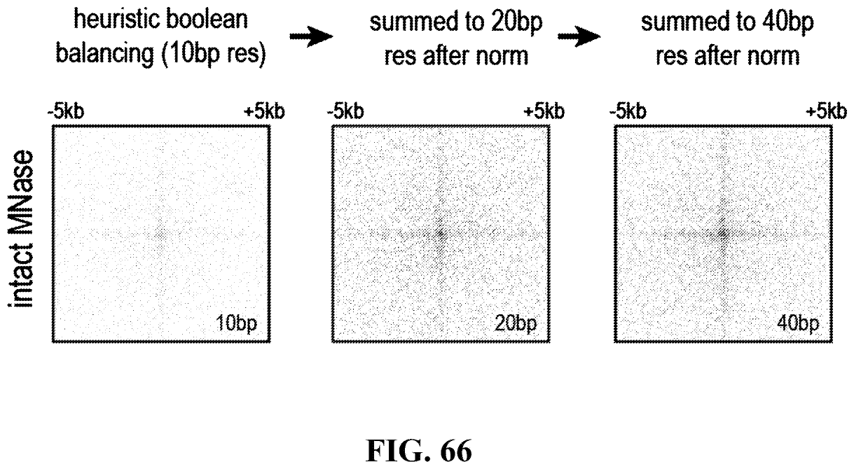

Applicants can visualize contacts using different methods. Boolean balancing presents an alternative approach to help with visualization (FIG. 66).

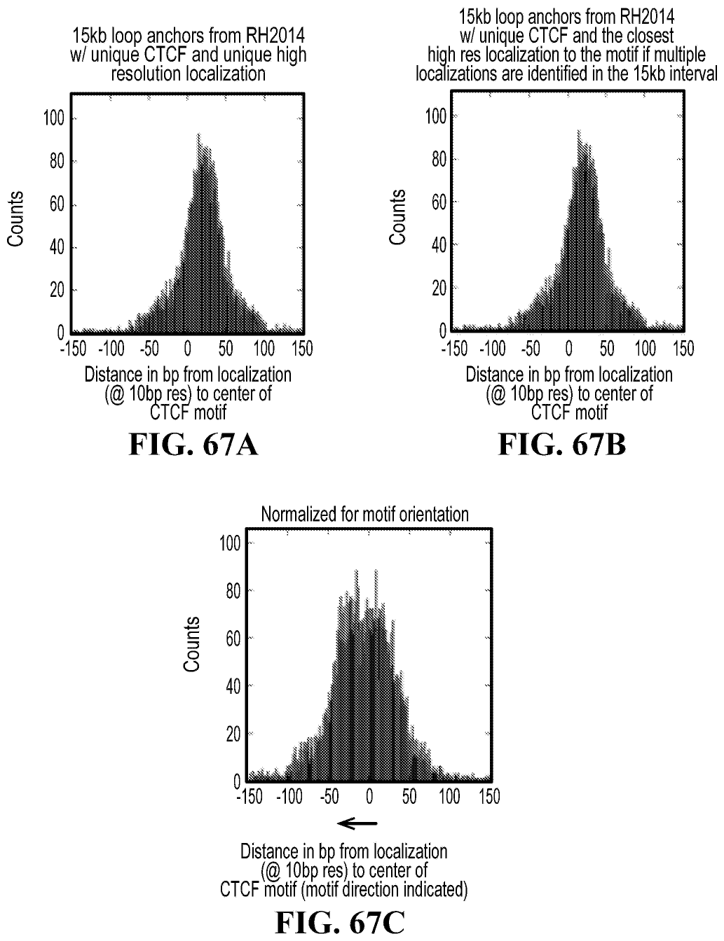

Applicants analyzed localizations around loop anchors with unique CTCF motifs (FIG. 67A-C). Out of 7058 15 kb loop anchors from RH2014 with a uniquely identified responsible CTCF motif, 5259 (75%) have a single high-resolution (@ 10 bp res) localization. Of these 5259 high-res localizations, 5162 (98.2%) overlap the motif (FIG. 67A). Out of 7058 15 kb loop anchors from RH2014 with a uniquely identified responsible CTCF motif, 6953 (98.5%) have at least one high-resolution (@ 10 bp res) localization. Of these 6953 localizations, 6785 (97.6%) overlap the motif (FIG. 67B). Applicants show contacts around unique CTCF motifs using the high resolution of intact Hi-C (10 bp resolution) and show that the localizations overlap the motifs and are centered at about 50 base pairs from the center of the motifs (FIG. 67A,B). When the graph is normalized for motif orientation the localizations are centered at about 50 base pairs from where the N-terminus of CTCF would be when bound to the motif (FIG. 67C). This localization is not possible with 1 kb resolution in in situ Hi-C.

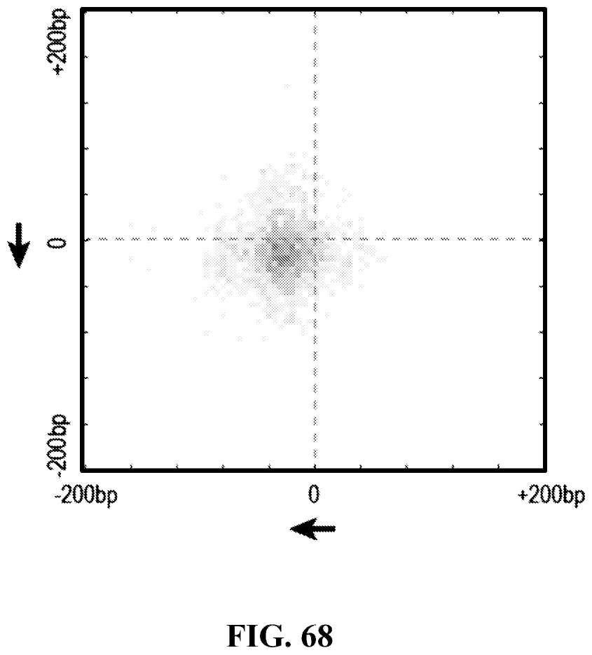

Applicants analyzed convergent CTCF loops from RH2014. Out of 2574 convergent CTCF loops from RH2014 with both responsible CTCF motifs identified, 2524 (98%) have high resolution localizations overlapping both of the coarse 15 kb loop anchors from RH2014 (FIG. 68). Of these 2524 high-res 2D localizations, 2449 (97%) overlap the convergent motif pair. The localizations tend to preferentially be in the interior of the loop (FIG. 68). Thus, cohesin may be sitting on top of CTCF to arrest cohesin.

Applicants have identified that the loop anchor is formed inside of convergent CTCF motifs. Thus, CTCF arrests cohesin and the loop anchor is formed about 50 bases from the CTCF motif. Applicants can now determine the location on an exact sequence of a bound protein arresting cohesin at CTCF independent loops. Thus, specific sequences or binding motifs can be identified. Further, once the sequence is determined, chromatin modifications or transcription factors can be mapped to the sequence based on ChIP data. This is not possible with in situ Hi-C because any motifs would be located somewhere within a 1 kb sequence.

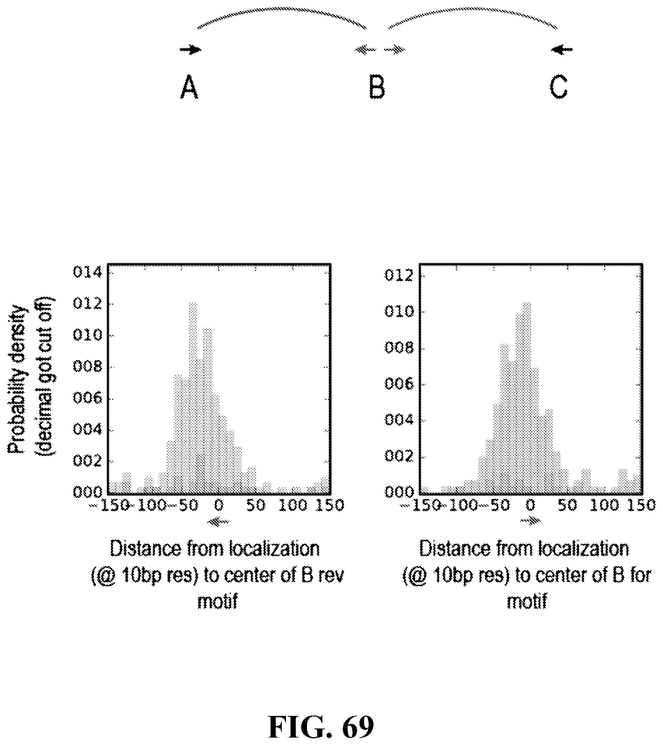

Applicants asked whether localizations are driven by 1D coverage biases using a locus that includes AB convergent CTCF motifs and BC convergent CTCF motifs (FIG. 69). Further, Applicants show that intact Hi-C be used to properly identify the responsible CTCF when the middle loci are very close together (e.g., hundreds of bp to several hundred kb apart). The localizations for B coming from localization running on the AB loops are shown (left) and the localizations for B coming from localization running on the BC loops are shown (right). This is a true 2D localization because it would be impossible for this to be driven by coverage biases.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. An in situ method for detecting spatial proximity relationships between genomic DNA in a cell with base pair resolution, comprising:
providing a sample of one or more cells;
crosslinking the cells with a chemical crosslinker;
lysing the cells to obtain isolated nuclei;
permeabilizing the nuclei under conditions that preserve cohesin complex integrity in the crosslinked cells;
enzymatically fragmenting the chromatin present in the nuclei;
performing end repair and/or fill-in on the ends of the chromatin fragments with at least one labeled nucleotide, wherein the labeled nucleotide is capable of being used to isolate the chromatin fragments;
ligating the repaired and/or filled in ends of the chromatin fragments that are in close physical proximity to create one or more end joined nucleic acid fragments having one or more junctions, wherein the site of the one or more junctions comprises one or more labeled nucleic acids;
reversing the crosslinking;
isolating the one or more end joined nucleic acid fragments using the labeled nucleotide; and
sequencing at the one or more junctions of the one or more end joined nucleic acid fragments by using ligation junction sequencing, thereby detecting spatial proximity relationships between genomic DNA in a cell.

2. The method of claim 1, wherein the steps of enzymatically fragmenting the chromatin present in the nuclei, performing end repair and/or fill-in on the ends of the chromatin fragments with at least one labeled nucleotide, and ligating the repaired and/or filled in ends of the chromatin fragments comprise:
a. a serial process comprising:
i. digesting the chromatin with a first restriction enzyme;
ii. filling in the overhanging ends produced from (i);
iii. ligating the filled in end of the chromatin fragments from (ii);
iv. digesting the chromatin fragments from (iii) with a second restriction enzyme;
v. filling in the overhanging ends produced from (iv); and
vi. ligating the filled in end of the chromatin fragments from (v);
b. a single-step process comprising:
i. in a single-step, fragmenting the chromatin present in the cells by contacting the chromatin with two restriction enzymes, filling in one or more overhanging ends of the chromatin fragments, and ligating two or more filled in ends;
c. a parallel process comprising:
i. fragmenting the chromatin present in the cell with two restriction enzymes in the same or parallel reactions;
ii. filling in the overhanging ends from (i), wherein the optional parallel reaction are optionally combined; and
iii. ligating two or more filled ends from (ii), wherein the optional parallel reaction are optionally combined;
d. a first MNase process comprising:
i. fragmenting the chromatin using micrococcal nuclease (MNase);
ii. repairing one or more overhanging ends produced in (i);
iii. filling in one or more repaired overhanging ends from (ii);
iv. ligating two or more filled ends from (iii);
e. a second MNase process comprising:
i. fragmenting the chromatin present in the cells with MNase;
ii. in a single step, repairing one or more ends of the chromatin fragments from (i), filling in one or more repaired overhanging ends from (ii), and ligating two or more filled ends from (i); or
f. a third MNase process comprising:
i. in a single step, fragmenting the chromatin present in the cells with MNase, repairing one or more ends of the chromatin fragments, filling in one or more repaired overhanging ends, and ligating two or more filled ends.

3. The method of claim 1 or 2, wherein ligation junction sequencing comprises selecting and sequencing approximately 250 base pair fragments using paired end sequencing.

4. The method of claim 1 or 2, wherein ligation junction sequencing comprises selecting and sequencing approximately 300 base pair fragments from a single end.

5. The method of any of claims 1 to 4, wherein the nuclei are permeabilized by a method comprising NP40, digitonin, tween, streptolysin, exonuclease 1 buffer and pepsin, cationic lipids, hypotonic shock, or ultrasonication; and wherein SDS is not used.

6. The method of any of claims 1 to 5, further comprising determining the sequence of a loop anchor with at least 10 base pair resolution.

7. The method of claim 6, further comprising identifying a sequence motif bound by a protein within 50 base pairs outside of the loop anchor.

8. The method of claim 7, wherein a promoter element bound by an RNA polymerase is identified.

9. The method of claim 7, wherein an enhancer motif bound by a transcription factor is identified.

10. The method of any of claims 1 to 9, further comprising identifying CTCF independent loops wherein cohesin is arrested by a factor other than CTCF.

11. The method of claim 10, wherein cohesin is arrested by an RNA polymerase or a transcription factor.

12. The method of any of claims 1 to 11, wherein promoter/enhancer loops are identified.

13. The method of claim 12, further comprising identifying sequence variants in an enhancer element and linking the variant to a gene.

14. The method of any of claims 1 to 13, further comprising determining the whole genome sequence for the cell based on the determined sequence information.

15. The method of any of claims 1 to 13, further comprising determining the whole exome sequence for the cell by enriching for exome sequences in the joined DNA fragments.

16. An in situ method for detecting spatial proximity relationships between genomic DNA in in a cell, comprising:
providing a sample of one or more cells;
crosslinking the cells with a chemical crosslinker;
lysing the cells to obtain isolated nuclei;
permeabilizing the nuclei;
enzymatically fragmenting the chromatin present in the nuclei;
performing end repair and/or fill-in on the ends of the chromatin fragments with at least one labeled nucleotide, wherein the labeled nucleotide is capable of being used to isolate the chromatin fragments;
ligating the repaired and/or filled in ends of the chromatin fragments that are in close physical proximity to create one or more end joined nucleic acid fragments having one or more junctions, wherein the site of the one or more junctions comprises one or more labeled nucleic acids;
reversing the crosslinking;
isolating the one or more end joined nucleic acid fragments using the labeled nucleotide; and
sequencing at the one or more junctions of the one or more end joined nucleic acid fragments, thereby detecting spatial proximity relationships between genomic DNA in a cell,
wherein the steps of enzymatically fragmenting the chromatin present in the nuclei, performing end repair and/or fill-in on the ends of the chromatin fragments with at least one labeled nucleotide, and ligating the repaired and/or filled in ends of the chromatin fragments comprise:
a. a serial process comprising:
i. digesting the chromatin with a first restriction enzyme;
ii. filling in the overhanging ends produced from (i);
iii. ligating the filled in end of the chromatin fragments from (ii);
iv. digesting the chromatin fragments from (iii) with a second restriction enzyme;
v. filling in the overhanging ends produced from (iv); and
vi. ligating the filled in end of the chromatin fragments from (v);
b. a single-step process comprising:
i. in a single-step, fragmenting the chromatin present in the cells by contacting the chromatin with two restriction enzymes, filling in one or more overhanging ends of the chromatin fragments, and ligating two or more filled in ends;
c. a parallel process comprising:
i. fragmenting the chromatin present in the cell with two restriction enzymes in the same or parallel reactions;
ii. filling in the overhanging ends from (i), wherein the optional parallel reaction are optionally combined; and
iii. ligating two or more filled ends from (ii), wherein the optional parallel reaction are optionally combined;
d. a first MNase process comprising:
i. fragmenting the chromatin using micrococcal nuclease (MNase);
ii. repairing one or more overhanging ends produced in (i);
iii. filling in one or more repaired overhanging ends from (ii);
iv. ligating two or more filled ends from (iii);
e. a second MNase process comprising:
i. fragmenting the chromatin present in the cells with MNase;
ii. in a single step, repairing one or more ends of the chromatin fragments from (i), filling in one or more repaired overhanging ends from (ii), and ligating two or more filled ends from (i); or
f. a third MNase process comprising:
i. in a single step, fragmenting the chromatin present in the cells with MNase, repairing one or more ends of the chromatin fragments, filling in one or more repaired overhanging ends, and ligating two or more filled ends.

17. The method of claim 16, wherein short-read sequencing technologies are used to determine the sequence at the one or more junctions of the one or more end joined nucleic acid fragments.

18. The method of claim 16, wherein long-read sequencing technologies are used to determine the sequence at the one or more junctions of the one or more end joined nucleic acid fragments.

19. The method of any of claims 1 to 18, further comprising assembling a whole genome or partial genome from the determined sequence information.

20. The method of claim 19, wherein the genome is assembled de novo.

21. The method of any of claims 1 to 20, further comprising assembling a fully phased diploid whole genome, partial phased genome, phased variant, or individual haplotype from the determined sequence information.

22. The method of claim 21, wherein sequence variants are assigned to single chromosomes.

23. The method of claim 21 or 22, wherein the method of phasing different haplotypes comprises calculating the frequency of contact between loci containing particular variants, wherein the frequency of contact between two variants indicates if two variants are on the same molecule.

24. The method of claim 23, wherein the variants are phased, and wherein phasing is determined, at least in part, based on the relative orientation with which a given variant forms contacts with other sequences in the set.

25. The method of claim 24, wherein the orientation is inner, outer, left, or right.

26. The method of claim 23, wherein the frequency of contact between two variants is compared to an expected model to determine whether the two variants are on a same molecule.

27. The method of claim 23, wherein the frequency of contact between two variants is compared to an expected model to determine whether the two variants are on sister chromatids.

28. The method of claim 26 or 27, wherein the expected model is determined based on a contact matrix derived from a DNA proximity ligation assay.

29. The method of any of claims 21 to 28, wherein the analysis is performed in an iterative fashion, and wherein data from DNA proximity ligation experiments is used to go from one possible phasing of a variant set to another possible phasing of a variant set.

30. The method of claim 29, wherein analysis of the data from the DNA proximity ligation experiments is performed using gradient descent, hill-climbing, a genetic algorithm, reducing to an instance of the Boolean satisfiability problem (SAT) and solving, or using any combinatorial optimization algorithm.

31. The method of any of claims 21 to 30, wherein the variants to be phased are derived from a single organism or multiple organisms.

32. The method of claim 31, wherein the multiple organisms are from the same species or a different species.

33. The method of any of claims 1 to 32, wherein the cells and/or cell nuclei are not subjected to mechanical lysis.

34. The method of any of claims 1 to 33, wherein the sample is not subjected to RNA degradation.

35. The method of any of claims 1 to 34, wherein the sample is not contacted with an exonuclease for removal of biotin from unligated ends.

36. The method of any of claims 1 to 35, wherein the sample is not subjected to phenol/chloroform extraction.

37. The method of any of claims 1 to 36, wherein fragmenting the nucleic acid present in the one or more cells comprises enzymatic digestion with an endonuclease that leaves 5′ overhanging ends.

38. The method of any of claims 1 to 37, wherein the chemical crosslinker comprises an aldehyde.

39. The method of claim 38, wherein the aldehyde comprises formaldehyde.

40. The method of any of claims 1 to 39, wherein reversing the crosslinking comprises contacting the sample with Proteinase K at elevated temperature.

41. The method of any of claims 1 to 40, wherein the labeled nucleotide is isolated with a specific binding agent that specifically binds to the label.

42. The method of any of claims 1 to 41, wherein the nucleotide is labeled with biotin.

43. The method of claim 41 or 42, wherein the specific binding agent comprises avidin and/or streptavidin.

44. The method of any of claims 41 to 43, wherein the specific binding agent is attached to a solid surface.

45. The method of any of claims 1 to 44, further comprising attaching sequencing adapters to the ends of the end joined nucleic acid fragments.

46. The method of any of claims 1 to 45, further comprising treating the sample with one or more agents prior to performing a PCR amplification step.

47. The method of claim 46, where the sample is treated with bisulfate or another chemical reagent that preserves DNA methylation information.

48. The method of any of claims 1 to 47, wherein the cells are cell cycle synchronized.

49. The method of claim 48, wherein the cells in the sample are synchronized in metaphase.

50. The method of any of claims 1 to 49, wherein the sample comprises cells obtained from a diseased tissue.

51. The method of any of claims 1 to 50, wherein the sample comprises cells obtained from a primary tissue.

52. The method of claim 51, wherein the primary tissue is blood.

53. The method of any of claims 1 to 52, wherein the sample is treated with an agent that isolates all end joined nucleic acids containing a specific nucleic acid sequence.

54. The method of claim 53, wherein the agent is a probe that specifically binds a specific nucleic acid sequence in the one or more junctions.

55. The method of claim 54, wherein the specific nucleic acid sequence is at least 120 base pairs long.

56. The method of claim 55, wherein the specific nucleic acid sequence is within at least 80 base pairs of a restriction site.

57. The method of claim 56, wherein the specific nucleotide sequence has less than 10 repetitive bases.

58. The method of claim 57, wherein the specific nucleic acid sequence has a GC content of between 25% and 80%.

59. The method of any of claims 54 to 58, wherein the probe is labeled.

60. The method of claim 59, wherein the probe is radiolabeled, fluorescently-labeled, biotin-labeled, enzymatically-labeled, or chemically-labeled.

61. The method of any of claims 54 to 60, wherein the probe is a RNA probe, a DNA probe, a locked nucleic acid (LNA) probe, a peptide nucleic acid (PNA) probe, or a hybrid RNA-DNA probe.

62. The method of any of claims 1 to 61, further comprising inferring or determining the three-dimensional structure of a genome comprising determining the sequence of the one or more junctions of the one or more end joined nucleic acid sequences and assembling the three-dimensional structure from the determined sequence information.

63. The method of claim 62, further comprising mapping protein-DNA interactions, chromatin post-translational modifications, or RNA-DNA interactions on the three-dimensional structure of the genome.

64. The method of claim 63, wherein protein DNA protein-DNA interactions and/or chromatin post-translational modifications are determined by chromatin immunoprecipitation sequencing (ChIP-seq).

65. The method of any of claims 1 to 64, further comprising simultaneous mapping of DNA methylation on the three-dimensional structure.

66. The method of any of claims 1 to 65, further comprising distinguishing between heterozygous and homozygous structural variations in samples based at least in part on the determined sequence information.

67. The method of any of claims 1 to 65, further comprising resolving the structural variation based at least in part on the determined sequence information.

68. The method of claim 67, wherein the structural variation resolved is a copy number variation.

69. A method of mapping complex genomic rearrangements comprising the method of any one of claims 1 to 68.

70. The method of claim 69, wherein the complex genomic rearrangements are the result of chromothripsis.

71. The method of claim 69 or 70, wherein the method comprises determining one or more breakpoints in the genomic sequence.

72. The method of any of claims 69 to 71, further comprising generating an end-to-end structure of a rearranged chromosome.

73. A method of diagnosing cancer comprising a method as in any one of claims 69 to 72.


SEQUENCE LISTING (39 sequences)

SEQ ID NO: 1 - protein, 288 aa, Artificial Sequence
  Synthetic N-terminal capping region
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 2 - protein, 183 aa, Artificial Sequence
  Synthetic C-terminal capping region
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 3 - protein, 7 aa, Simian virus 40
PKKKRKV

SEQ ID NO: 4 - protein, 10 aa, Simian virus 40
PKKKRKVEAS

SEQ ID NO: 5 - protein, 16 aa, Artificial Sequence
  Synthetic nucleoplasmin bipartite NLS
KRPAATKKAGQAKKKK

SEQ ID NO: 6 - protein, 9 aa, Homo sapiens
PAAKRVKLD

SEQ ID NO: 7 - protein, 11 aa, Homo sapiens
RQRRNELKRSP

SEQ ID NO: 8 - protein, 38 aa, Homo sapiens
NQSSNFGPMKGGNFGGRSSGPYGGGGQYFAKPRNQGGY

SEQ ID NO: 9 - protein, 42 aa, Homo sapiens
RMRIZFKNKGKDTAELRRRRVEVSVELRKAKKDEQILKRRNV

SEQ ID NO: 10 - protein, 8 aa, Homo sapiens
VSRKRPRP

SEQ ID NO: 11 - protein, 8 aa, Homo sapiens
PPKKARED

SEQ ID NO: 12 - protein, 8 aa, Homo sapiens
PQPKKKPL

SEQ ID NO: 13 - protein, 12 aa, Mus sp.
SALIKKKKKMAP

SEQ ID NO: 14 - protein, 5 aa, Influenza virus
DRLRR

SEQ ID NO: 15 - protein, 7 aa, Influenza virus
PKQKKRK

SEQ ID NO: 16 - protein, 10 aa, Hepatitis D virus
RKLKKKIKKL

SEQ ID NO: 17 - protein, 10 aa, Mus sp.
REKKKFLKRR

SEQ ID NO: 18 - protein, 20 aa, Homo sapiens
KRKGDEVDGVDEVAKKKSKK

SEQ ID NO: 19 - protein, 17 aa, Homo sapiens
RKCLQAGMNLEARKTKK

SEQ ID NO: 20 - DNA, 14 bp, Artificial Sequence
  Synthetic
  misc_feature (5)..(5): n is a, c, g, or t
ccacnaggtggcag

SEQ ID NO: 21 - DNA, 150 bp, Artificial Sequence
  Synthetic
  misc_feature (16)..(135): n is a, c, g, or t
tcgcgcccataactcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctgagggtccgcctt

SEQ ID NO: 22 - DNA, 150 bp, Artificial Sequence
  Synthetic
  misc_feature (16)..(135): n is a, c, g, or t
atcgcaccagcgtgtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncactgcggctcctca

SEQ ID NO: 23 - DNA, 150 bp, Artificial Sequence
  Synthetic
  misc_feature (16)..(135): n is a, c, g, or t
cctcgcctatcccatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncactaccggggtctg

SEQ ID NO: 24 - DNA, 21 bp, Artificial Sequence
  Synthetic
ctgggatcgcgcccataactc

SEQ ID NO: 25 - DNA, 21 bp, Artificial Sequence
  Synthetic
ctgggaatcgcaccagcgtgt

SEQ ID NO: 26 - DNA, 21 bp, Artificial Sequence
  Synthetic
ctgggacctcgcctatcccat

SEQ ID NO: 27 - DNA, 21 bp, Artificial Sequence
  Synthetic
cgtggaaaggcggaccctcag

SEQ ID NO: 28 - DNA, 21 bp, Artificial Sequence
  Synthetic
cgtggatgaggagccgcagtg

SEQ ID NO: 29 - DNA, 21 bp, Artificial Sequence
  Synthetic
cgtggacagaccccggtagtg

SEQ ID NO: 30 - DNA, 41 bp, Artificial Sequence
  Synthetic
ggattctaatacgactcactatagggtcgcgcccataactc

SEQ ID NO: 31 - DNA, 41 bp, Artificial Sequence
  Synthetic
ggattctaatacgactcactatagggatcgcaccagcgtgt

SEQ ID NO: 32 - DNA, 40 bp, Artificial Sequence
  Synthetic
ggattctaatacgactcactatagggcctcgcctatccca

SEQ ID NO: 33 - DNA, 14 bp, Artificial Sequence
  Synthetic
  misc_feature (10)..(10): n is a, c, g, or t
ctgccacctngtgg

SEQ ID NO: 34 - DNA, 30 bp, Artificial Sequence
  Synthetic
gagcaattccgccccctggtggcagatctg

SEQ ID NO: 35 - DNA, 14 bp, Artificial Sequence
  Synthetic
  misc_feature (5)..(5): n is a, c, g, or t
ccacnaggtggcag

SEQ ID NO: 36 - DNA, 30 bp, Artificial Sequence
  Synthetic
ggcggagaccacaaggtggcgccagatccc

SEQ ID NO: 37 - DNA, 15 bp, Artificial Sequence
  Synthetic
tggaagaaaccgttt

SEQ ID NO: 38 - DNA, 12 bp, Artificial Sequence
  Synthetic
aggaccgagttt

SEQ ID NO: 39 - DNA, 17 bp, Artificial Sequence
  Synthetic
tgtaagaaaccgagttt